(19) United States
(12) Patent Application Publication
(10) Pub. No.: US 2004/0034222 A1
(43) Pub. Date: Feb. 19, 2004

(54) 2,4 diamino-1,3,5-triazines, processes for their preparation and their use as herbicides and plant growth regulators

(76) Inventors: Jens Hollander, Schmitten (DE); Lothar Willms, Hofheim (DE); Jurgen Zindel, Bad Sooden-Allendorf (DE); Wolfgang Giencke, Hofheim (DE); Hermann Bieringer, Eppstein (DE); Christopher Rosinger, Hofheim (DE); Thomas Auler, Bad Soden (DE); Felix Thurwachter, Bad Homburg (DE)

Correspondence Address: FROMMER LAWRENCE & HAUG, 745 FIFTH AVENUE- 10TH FL., NEW YORK, NY, 10151, US

(21) Appl. No.: 10/354,618

(22) Filed: Jan. 30, 2003

Related U.S. Application Data
(63) Continuation of application No. 09/527,119, filed on Mar. 16, 2000, now abandoned.

(30) Foreign Application Priority Data
Mar. 20, 1999 (DE) 199 12 637.2

(51) Int. Cl. C 07D 4 3/02; C07D 251/42

(52) U.S. Cl. 544/211; 544/212

(57) ABSTRACT

Compounds of the formula (I) or salts thereof

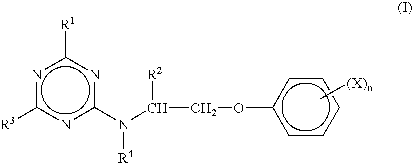

in which

R¹=(subst.)aryl, (subst.)(C₃-C₉)cycloalkyl, (subst.)heterocyclyl, (subst.)(C₁-C₆)alkyl, (subst.)(C₂-C₆)alkenyl or (subst.)(C₂-C₆)alkynyl,

R²=a (subst.)carbocycle having 4 to 6 ring atoms, (subst.)heterocycle having 3 to 6 ring atoms and 1 to 4 heteroatoms N, O and S, or the formula —A—Z, in which A=straight-chain unsubst. or subst. (C₁-C₄)alkylene, (C₂-C₅)alkenylene or (C₂-C₅)alkynylene, and Z=(subst.)carbocycle having 3-6 ring atoms, (subst.)heterocycle having 3-6 ring atoms and 1-4-heteroatoms N, O and S,

R³=H, (subst.)(C₁-C₆)alkyl, (subst.)aryl or (subst.)(C₃-C₉)cycloalkyl, or a radical of the formula —N(B¹—D¹)(B²—D²) or —NR′—N(B¹—D¹)(B²—D²), in which B¹, B², D¹ and D² are as defined below, and R′=H, (C₁-C₆)alkyl or [(C₁-C₄)alkyl]carbonyl,

R⁴=—B³—D³, where B³ and D³ are as defined below,

B¹, B² and B³=a direct bond or —C(═Z*)—, —C(═Z*)—Z**—, —C(═Z*)—NH— or —C(═Z*)—NR*—, where Z*, Z**=O, S and R*=(C₁-C₆)alkyl, aryl, aryl-(C₁-C₆)alkyl, (C₃-C₉)cycloalkyl or (C₃-C₉)cycloalkyl-(C₁-C₆)alkyl, each case opt. subst., D¹, D², D³=H, (C₁-C₆)alkyl, aryl, aryl-(C₁-C₆)alkyl, (C₃-C₉)cycloalkyl or (C₃-C₉)cycloalkyl-(C₁-C₆)alkyl, in each case opt. substituted, and

(X)ₙ are substituents as defined in more detail in claim 1 and n=0-5, are suitable for use as herbicides and plant growth regulators.

### DESCRIPTION

The invention relates to the technical field of the crop protection agents, such as herbicides and plant growth regulators, in particular herbicides for the selective control of harmful plants in crops of useful plants.

It ist known that 2-amino4-(N-phenoxyalkylamino)-1,3,5-triazines which are substituted in the 6 position and may be substituted further, have herbicidal and plant-growth-regulating properties; cf. WO98/15537, DE-A-19641691, WO98/34925 and literature cited therein.

Some of the known active compounds have, when used, disadvantages, such as insufficient herbicidal activity against harmful plants, too limited a spectrum of harmful plants which can be controlled with an active compound, or insufficient selectivity in crops of useful plants. Other active compounds cannot be prepared economically on an industrial scale, owing to reagents and precursors which are difficult to obtain, or they have insufficient chemical stability.

It is an object of the invention to provide alternative active compounds of the type of the 2,4-diamino-1,3,5-triazines which can, if appropriate, advantageously, be employed as herbicides or plant growth regulators.

The present invention provides compounds of the formula (I) and salts thereof

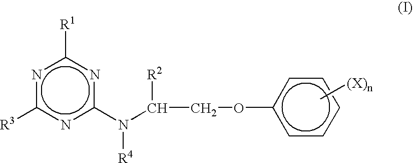

in which

R¹ is aryl, which is unsubstituted or substituted and, including substituents, has preferably 6 to 30 carbon atoms, or (C₃-C₉)cycloalkyl, which is unsubstituted or substituted and, including substituents, has preferably 3 to 30 carbon atoms, or heterocyclyl, which is substituted or unsubstituted and, including substituents, has preferably 2 to 30 carbon atoms, or

is (C₁-C₆)alkyl, (C₂-C₆)alkenyl or (C₂-C₆)alkynyl, where each of the 3 lastmentioned radicals is unsubstituted or substituted by one or more radicals selected from the group consisting of halogen, hydroxyl, cyano, nitro, thiocyanato, (C₁-C₄)alkoxy, (C₁-C₄)haloalkoxy, (C₂-C₄)alkenyloxy, (C₂-C₄)haloalkenyloxy, (C₁-C₄)alkylthio, (C₁-C₄)alkylsulfinyl, (C₁-C₄)alkylsulfonyl, (C₁-C₄)haloalkylsulfinyl, (C₁-C₄)haloalkyl-sulfonyl and (C₃-C₉)cycloalkyl, which is unsubstituted or substituted, and phenyl, which is unsubstituted or substituted, and heterocyclyl, which is unsubstituted or substituted, and radicals of the formulae R′—C(═Z′)—, R′—C(═Z′)—Z—, R′—Z—C(═Z′)—, R′R″N—C(═Z′)—, R′—Z—C(═Z′)—O—, R′R″N—C(═Z′)—Z—, R′—Z—C(═Z′)—NR″— and R′R″N—C(═Z′)—NR′″—, in which R′, R″ and R′″, independently of one another, are in each case (C₁-C₆)alkyl, aryl, aryl-(C₁-C₆)alkyl, (C₃-C₉)cycloalkyl or (C₃-C₉)cycloalkyl-(C₁-C₆)alkyl, where each of the 5 lastmentioned radicals is unsubstituted or substituted, and in which Z and Z′ independently of one another are each an oxygen or sulfur atom,

and, including substituents, has preferably 1 to 30 carbon atoms,

R² is a carbocyclic saturated, partially unsaturated or aromatic radical having 4 to 6 ring atoms or a heteroaromatic, saturated or partially unsaturated heterocyclic radical which contains 3 to 6 ring atoms and 1 to 4 heteroatoms selected from the group N, O and S in the heterocyclic ring, where each of the cyclic radicals mentioned is unsubstituted or substituted and, including substituents, has preferably up to 30 carbon atoms, or

is a radical of the formula —A—Z, in which

A is straight-chain alkylene having 1 to 4 carbon atoms or straight-chain alkenylene or alkynylene having in each case 2 to 5 carbon atoms, where each of the three lastmentioned diradicals is unsubstituted or substituted by one or more radicals selected from the group consisting of halogen, nitro, cyano, thiocyanato, (C₁-C₄)alkyl, (C₁-C₄)alkoxy, (C₁-C₄)alkylthio, (C₁-C₄)haloalkyl and (C₁-C₄)haloalkoxy, and

Z is a carbocyclic saturated, partially unsaturated or aromatic radical having 3 to 6 ring atoms or a heteroaromatic, saturated or partially unsaturated heterocyclic radical which contains 3 to 6 ring atoms and 1 to 4 heteroatoms selected from the group consisting of N, O and S in the heterocyclic ring, where each of the cyclic radicals mentioned is unsubstituted or substituted and, including substituents, has preferably up to 30 carbon atoms,

R³ is hydrogen, (C₁-C₆)alkyl, aryl or (C₃-C₉)cycloalkyl, where each of the 3 lastmentioned radicals is unsubstituted or substituted, or is a radical of the formula —N(B¹—D¹)(B²—D²) or —NR′—N(B¹—D¹)(B²—D²), where B¹, B², D¹ and D² are each as defined below and R′=hydrogen, (C₁-C₆)alkyl or [(C₁-C₄)alkyl]carbonyl, where R³, including substituents, has preferably up to 20 carbon atoms,

R⁴ is a radical of the formula —B³—D³, where B³ and D³ are as defined below and R⁴, including substituents, has preferably up to 20 carbon atoms,

B¹, B² and B³ independently of one another are each a direct bond or a divalent group of the formula —C(═Z*)—, —C(═Z*)—Z**—, —C(═Z*)—NH— or —C(═Z*)—NR*—, where Z*=an oxygen or sulfur atom, Z*=an oxygen or sulfur atom and R*=(C₁-C₆)alkyl, aryl, aryl-(C₁-C₆)alkyl, (C₃-C₉)cycloalkyl or (C₃-C₉)cycloalkyl-(C₁-C₆)alkyl, where each of the 5 lastmentioned radicals is unsubstituted or substituted and, including substituents, has prefrably up to 20 carbon atoms,

D¹, D², and D³ independently of one another are each hydrogen, (C₁-C₆)alkyl, aryl, aryl-(C₁-C₆)alkyl, (C₃-C₉)cycloalkyl or (C₃-C₉)cycloalkyl-(C₁-C₆)alkyl, where each of the 5 last mentioned radicals is unsubstituted or substituted and, including substituents, has preferably up to 20 carbon atoms,

(X)ₙ are n substituents X, and X, independently of the others is in each case halogen, hydroxyl, amino, nitro, formyl, carboxyl, cyano, thiocyanato, aminocarbonyl or (C₁-C₆)alkyl, (C₁-C₆)alkoxy, (C₁-C₆)alkylthio, mono(C₁-C₆)alkylamino, di(C₁-C₄)alkylamino, (C₂-C₆)alkenyl, (C₂-C₆)alkynyl, [(C₁-C₆)alkyl]carbonyl, [(C₁-C₆)-alkoxy]carbonyl, mono(C₁-C₆)alkylaminocarbonyl, di(C₁-C₄)alkyl-aminocarbonyl, N-(C₁-C₆)alkanoylamino or N—(C₁-C₄)alkanoyl-N-(C₁-C₄)alkylamino,

where each of the 13 lastmentioned radicals is unsubstituted or substituted, preferably unsubstituted or substituted by one or more radicals selected from the group consisting of halogen, hydroxyl, amino, nitro, formyl, carboxyl, cyano, thiocyanato, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, (C₃-C₉)cycloalkylamino, [(C₁-C₄)alkyl]carbonyl, [(C₁-C₄)alkoxy]-carbonyl, aminocarbonyl, mono(C₁-C₄)alkylaminocarbonyl, di(C₁-C₄)alkylaminocarbonyl, phenyl, phenoxy, phenylthio, phenylcarbonyl, heterocyclyl, heterocyclyloxy, heterocyclylthio and heterocyclylamino,

where each of the 8 lastmentioned radicals is unsubstituted or has one or more substituents selected from the group consisting of halogen, nitro, cyano, (C₁-C₄)alkyl, (C₁-C₄)alkoxy, (C₁-C₄)alkylthio, (C₁-C₄)haloalkyl, (C₁-C₄)haloalkoxy, formyl, (C₁-C₄)alkylcarbonyl and (C₁-C₄)alkoxycarbonyl,

or is (C₃-C₉)cycloalkyl, (C₃-C₉)cycloalkoxy, (C₃-C₉)cycloalkylamino, phenyl, phenoxy, phenylthio, phenylcarbonyl, heterocyclyl heterocyclyloxy, heterocyclylthio or heterocyclylamino,

where each of the 11 lastmentioned radicals is unsubstituted or substituted, preferably unsubstituted or substituted by one or more radicals selected from the group consisting of halogen, hydroxyl, amino, nitro, formyl, carboxyl, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cyclo-alkyl, [(C₁-C₄)alkyl]carbonyl, [(C₁-C₄)alkoxy]carbonyl, amino-carbonyl, mono(C₁-C₄)alkylaminocarbonyl and di(C₁-C₄)alkyl-aminocarbonyl,

or two adjacent radicals X together are a fused-on cycle having 4 to 6 ring atoms which is carbocyclic or contains hetero ring atoms selected from the group consisting of O, S and N and which is unsubstituted or substituted by one or more radicals selected from the group consisting of the halogen, (C₁-C₄)alkyl and oxo,

n is 0, 1, 2, 3, 4 or 5, preferably 0, 1, 2, 3 or 4, in particular 1 or 2, and

heterocyclyl in the radicals mentioned above independently of the others is in each case a heterocyclic radical having 3 to 7 ring atoms and 1 to 4 heteroatoms selected from the group consisting of N, O and S.

Unless defined otherwise, divalent radicals, for example B¹=—C(═Z*)—Z**—, are defined such that in the composed groups, for example —B¹—D¹, the bond of the divalent radical linked to the group D¹ is the bond which is written on the right in the formula for the divalent radical, i.e. —B¹—D¹ is a group of the formula —C(═Z*)—Z**—D¹; this applies correspondingly to analogous divalent radicals.

The compounds of the formula (I) can form salts by addition of a suitable inorganic or organic acid, such as, for example, HCl, HBr, H₂SO₄ or HNO₃, but also oxalic acid or sulfonic acids, to a basic group, such as, for example, amino or alkylamino. Suitable substituents which are present in deprotonated form, such as, for example, sulfonic acids or carboxylic acids, can form inner salts with groups which for their part can be protonated, such as amino groups. Salts can also be formed by replacing the hydrogen of suitable substituents, such as, for example, sulfonic acids or carboxylic acids, by an agriculturally suitable cation. These salts are, for example, metal salts, in particular alkali metal salts or alkaline earth metal salts, in particular sodium salts and potassium salts, or else ammonium salts, salts with organic amines or quaternary ammonium salts with cations of the formula [NRR′R″R′″]⁺, in which R to R′″ independently of one another are each an organic radical, in particular alkyl, aryl, aralkyl or alkylaryl. Hereinbelow, compounds of the formula (I) according to the invention and their salts are also abbreviated to “compounds (I)” according to the invention.”

In the formula (I) and all the formulae hereinbelow, the radicals alkyl, alkoxy, haloalkyl, haloalkoxy, alkylamino and alkylthio and the corresponding unsaturated and/or substituted radicals can in each case be straight-chain or branched in the carbon skeleton. Unless specifically mentioned otherwise, the lower carbon skeletons, for example with 1 to 6 carbon atoms, in particular 1 to 4 carbon atoms, or in the case of unsaturated groups with 2 to 6 carbon atoms, in particular 2 to 4 carbon atoms, are preferred for these radicals. Alkyl radicals, also in the composed meanings, such as alkoxy, haloalkyl, and the like, are, for example, methyl, ethyl, n- or isopropyl, n-, i-, t- or 2-butyl, pentyls, hexyls, such as n-hexyl, isohexyl and 1,3-dimethylbutyl, heptyls, such as n-heptyl, 1-methylhexyl and 1,4-dimethylpentyl; alkenyl and alkynyl radicals have the meaning of the possible unsaturated radicals which correspond to the alkyl radicals and contain at least one double bond and triple bond, respectively, preferably one double bond and triple bond, respectively. Alkenyl is, for example, allyl, 1-methylprop-2-en-1-yl, 2-methylprop-2-en-1-yl, but-2-en-1-yl, but-3-en-1-yl, 1-methyl-but-3-en-1-yl and 1-methyl-but-2-en-1-yl; alkynyl is, for example, propargyl, but-2-yn-1-yl, but-3-yn-1-yl, 1-methylbut-3-yn-1-yl.

Alkylidene, for example in the form (C₁-C₁₀)alkylidene, is the radical of a straight-chain or branched alkane which is attached via a double bond, the position of the binding site not yet being fixed. In the case of a branched alkane, the only possible positions are, of course, those where two hydrogen atoms can be replaced by the double bond; examples of radicals are ═CH₂, ═CH—CH₃, ═C(CH₃)—CH₃, ═C(CH₃)—C₂H₅ or ═C(C₂H₅)—C₂H₅.

Cycloalkyl is a carbocyclic saturated ring system having preferably 3-8 carbon atoms, preferably 3 to 6 carbon atoms, for example cyclopropyl, cyclobutyl, cyclopentyl or cyclohexyl. In the case of substituted cycloalkyl, this includes cyclic systems with substituents, where the substitutents are attached to the cycloalkyl radical via a double bond, for example an alkylidene group such as methylidene. Substituted cycloalkyl also includes polycyclic aliphatic systems, such as, for example, bicyclo[1.1.0]butan-1-yl, bicyclo[1.1.0]butan-2-yl, bicyclo[2.1.0]pentan-1-yl, bicyclo[2.1.0]pentan-2-yl, bicyclo[2.1.0]pentan-5-yl, adamantan-1-yl and adamantan-2-yl. Cycloalkenyl is a carbocyclic non-aromatic, partially unsaturated ring system having preferably 4-8 carbon atoms, in particular 5 to 7 carbon atoms, for example 1-cyclobutenyl, 2-cyclobutenyl, 1-cyclopentenyl, 2-cyclopentenyl, 3-cyclopentenyl, or 1-cyclohexenyl, 2-cyclohexenyl, 3-cyclohexenyl, 1,3-cyclohexadienyl or 1,4-cyclohexadienyl. For substituted cycloalkenyl, the illustrations for substituted cycloalkyl apply correspondingly.

Halogen is, for example, fluorine, chlorine, bromine or iodine. Haloalkyl, -alkenyl and -alkynyl is alkyl, alkenyl and alkynyl, respectively, which is partially or fully substituted by halogen, preferably by fluorine, chlorine and/or bromine, in particular by fluorine or chlorine, for example, monohaloalkyl, perhaloalkyl, CF₃, CHF₂, CH₂F, CF₃CF₂, CH₂FCHCl, CCl₃, CHCl₂, CH₂CH₂Cl; haloalkoxy is, for example, OCF₃, OCHF₂, OCH₂F, CF₃CF₂O, OCH₂CF₃ and OCH₂CH₂Cl; this applies correspondingly to haloalkenyl and other halogen-substituted radicals.

Aryl is a mono-, bi- or polycyclic aromatic system, for example phenyl, naphthyl, tetrahydronaphthyl, indenyl, indanyl, pentalenyl, fluorenyl and the like, preferably phenyl.

A heterocyclic radical or ring (heterocyclyl) can be saturated, unsaturated or heteroaromatic; unless defined otherwise, it preferably contains one or more, in particular 1, 2 or 3, heteroatoms in the heterocyclic ring, preferably selected from the group consisting of N, O and S; it is preferably an aliphatic heterocyclyl radical having 3 to 7 ring atoms or a heteroaromatic radical having 5 or 6 ring atoms. The heterocyclic radical can, for example, be a heteroaromatic radical or ring (heteroaryl), such as, for example, a mono-, bi- or polycyclic aromatic system, in which at least 1 ring contains one or more heteroatoms. It is preferably a heteroaromatic ring having one heteroatom selected from the group consisting of N, O and S, for example pyridyl, pyrrolyl, thienyl or furyl; furthermore, preferably, it is a corresponding heteroaromatic ring having 2 or 3 heteroatoms, for example pyrimidinyl, pyridazinyl, pyrazinyl, triazinyl, thiazolyl, thiadiazolyl, oxazolyl, isoxazolyl, pyrazolyl, imidazolyl and triazolyl. Furthermore preferably, it is a partially or fully hydrogenated heterocyclic radical having one heteroatom selected from the group consisting of N, O and S, for example oxiranyl, oxetanyl, oxolanyl (=tetrahydrofuryl), oxanyl, pyrrolidyl (=pyrrolidinyl) or piperidyl or else pyrrolinyl, such as Δ¹-pyrrolinyl, Δ²-pyrrolinyl or Δ³-pyrrolinyl, for example Δ¹-pyrrolin-2-yl, Δ¹-pyrrolin-3-yl, Δ¹-pyrrolin-4-yl or Δ¹-pyrrolin-5-yl or Δ²-pyrrolin-1-yl, Δ²-pyrrolin-2-yl, Δ²-pyrrolin-3-yl, Δ2-pyrrolin4-yl, Δ2-pyrrolin-5-yl or Δ³-pyrrolin-1-yl, Δ³-pyrrolin-2-yl or Δ³-pyrrolin-3-yl.

Furthermore preferably, it is a partially or fully hydrogenated heterocyclic radical having 2 heteroatoms selected from the group consisting of N, O and S, for example piperazinyl, dioxolanyl, boxazolinyl, isoxazolinyl, oxazolidinyl, isoxazolidinyl and morpholinyl.

Possible substituents for a substituted heterocyclic radical are the substituents mentioned further below, and additionally also oxo. The oxo group can also be present at the hetero ring atoms which can exist in different oxidation states, for example at N and S.

Substituted radicals, such as a substituted alkyl, alkenyl, alkynyl, aryl, phenyl, benzyl, heterocyclyl and, specifically, heteroaryl radical, are, for example, a substituted radical derived from the unsubstituted skeleton, where the substituents are, for example, one or more, preferably 1, 2 or 3, radicals selected from the group consisting of halogen, alkoxy, haloalkoxy, alkylthio, hydroxyl, amino, nitro, carboxyl, cyano, azido, alkoxycarbonyl, alkylcarbonyl, formyl, carbamoyl, mono- and dialkylaminocarbonyl, substituted amino, such as acylamino, mono- and dialkylamino, and alkylsulfinyl, haloalkylsulfinyl, alkylsulfonyl, haloalkylsulfonyl and, in the case of cyclic radicals, also alkyl and haloalkyl; the “substituted radicals”, such as in substituted alkyl and the like, includes as substituents, in addition to the saturated hydrocarbon-containing radicals mentioned, the corresponding unsaturated aliphatic and aromatic radicals, such as unsubstituted or substituted alkenyl, alkynyl, alkenyloxy, alkynyloxy, phenyl, phenoxy etc. Substituted cyclic radicals having aliphatic moieties in the ring include cyclic systems having substituents which are attached to the ring via a double bond, for example those substituted by an alkylidene group, such as methylidene or ethylidene.

The substituents mentioned by way of example (“first substituent level”) can, if they contain hydrocarbon-containing moieties, be, if appropriate, substituted further in these moieties (“second substituent level”), for example by one of the substituents defined for the first substituent level. Corresponding further substituent levels are possible. The term “substituted radical” preferably only embrances one or two substituent levels.

Preferred substituents for the substituent levels are, for example, amino, hydroxyl, halogen, nitro, cyano, mercapto, carboxyl, carboxamide, SF₅, aminosulfonyl, alkyl, cycloalkyl, alkenyl, cycloalkenyl, alkynyl, monoalkyl-amino, dialkylamino, n-alkanoylamino, alkoxy, alkenyloxy, alkynyloxy, cycloalkoxy, cycloalkenyloxy, alkoxycarbonyl, alkenyloxycarbonyl, alkynyloxycarbonyl, aryloxycarbonyl, alkanoyl, alkenyl-carbonyl, alkynyl-carbonyl, aryl-carbonyl, alkylthio, cycloalkylthio, alkenylthio, cycloalkenylthio, alkynylthio, alkylsulfinyl, alkylsulfonyl, monoalkyl-aminosulfonyl, dialkyl-aminosulfonyl, n-alkyl-aminocarbonyl, N, N-dialkyl-aminocarbonyl, N-alkanoyl-amino-carbonyl, N-alkanoyl-N-alkyl-aminocarbonyl, aryl, aryloxy, benzyl, benzyloxy, benzylthio, arylthio, arylamino and benzylamino.

Among the radicals with carbon atoms, preference is given to those having 1 to 6 carbon atoms, preferably 1 to 4 carbon atoms, in particular 1 or 2 carbon atoms. In general, preference is given to substituents selected from the group consisting of halogen, for example fluorine and chlorine, (C₁-C₄)alkyl, preferably methyl or ethyl, (C₁-C₄)haloalkyl, preferably trifluoromethyl, (C₁-C₄)alkoxy, preferably methoxy or ethoxy, (C₁-C₄)haloalkoxy, nitro and cyano. Particular preference is given here to the substituents methyl, methoxy and chlorine.

Mono- or disubstituted amino is a chemically stable radical from the group of the substituted amino radicals which are N-substituted, for example, by one or two identical or different radicals selected from the group consisting of alkyl, alkoxy, acyl and aryl; preferably monoalkylamino, dialkylamino, acylamino, arylamino, N-alkyl-N-arylamino and N-heterocycles; preference is given to alkyl radicals having 1 to 4 carbon atoms; aryl is preferably phenyl or substituted phenyl; for acyl, the definition mentioned further below applies, preferably (C₁-C₄)alkanoyl. This applies correspondingly to substituted hydroxylamino or hydrazino.

Unsubstituted or substituted phenyl is preferably phenyl which is unsubstituted or mono- or polysubstituted, preferably up to trisubstituted, by identical or different radicals selected from the group consisting of halogen, (C₁-C₄)alkyl, (C₁-C₄)alkoxy, (C₁-C₄)haloalkyl, (C₁-C₄)haloalkoxy and nitro, for example o-, m- and p-tolyl, dimethylphenyls, 2-, 3- and 4-chlorophenyl, 2-, 3- and 4-trifluoromethyl- and -trichloromethylphenyl, 2,4-, 3,5-, 2,5- and 2,3-dichlorophenyl, o-, m- and p-methoxyphenyl.

An acid radical of an inorganic or organic oxygen acid is a radical which is formally formed by removing a hydroxyl group from the acid function, for example the sulfo radical —SO₃H, which is derived from sulfuric acid H₂SO₄, or the sulfino radical —SO₂H, which is derived from sulfurous acid H₂SO₃, or, correspondingly, the group SO₂NH₂, the phospho radical —PO(OH)₂, the group —PO(NH₂)₂, —PO(OH)(NH₂), —PS(OH)₂, —PS(NH₂)₂ or —PS(OH)(NH₂), the carboxyl radical COOH, which is derived from carbonic acid, radicals of the formula —CO—SH, —CS—OH, —CS—SH, —CO—NH₂, —CS—NH₂, —C(═NH)—OH or —C(═NH)—NH₂; also possible are radicals with hydrocarbon radicals or substituted hydrocarbon radicals, i.e. acyl radicals in the widest sense (=“acyl”).

Acyl is a radical of an organic acid which is formally formed by removing a hydroxyl group from the acid function, where the organic radical in the acid can also be attached to the acid function via a heteroatom. Examples of acyl are the radical —CO—R of a carboxylic acid HO—CO—R and radicals of acids derived therefrom, such as thiocarbonic acid, unsubstituted or N-substituted iminocarboxylic acids or the radical of carbonic monoesters, N-substituted carbamic acid, sulfonic acids, sulfinic acids, N-substituted sulfonamide acids, phosphonic acids, phosphinic acids.

Acyl is, for example, formyl, alkylcarbonyl, such as [(C₁-C₄)alkyl]carbonyl, phenylcarbonyl, alkyloxycarbonyl, phenyloxycarbonyl, benzyloxycarbonyl, alkylsulfonyl, alkylsulfinyl, N-alkyl-1-iminoalkyl and other radicals of organic acids. The radicals can in each case be further substituted in the alkyl or phenyl moiety, for example in the alkyl moiety by one or more radicals selected from the group consisting of halogen, alkoxy, phenyl and phenoxy; examples of substituents in the phenyl moiety are the substituents which have already been mentioned further above generally for substituted phenyl. Acyl is preferably an acyl radical in the more restricted sense, i.e. a radical of an organic acid where the acid group is directly attached to the carbon atom of an organic radical, for example formyl, alkylcarbonyl, such as acetyl or [(C₁-C₄)alkyl]carbonyl, phenylcarbonyl, alkylsulfonyl, alkylsulfinyl and other radicals of organic acids.

The invention also provides all stereoisomers which are embraced by the formula (I) and mixtures thereof. Such compounds of the formula (I) contain one or more asymmetric carbon atoms (=asymmetrically substituted carbon atoms) or else double bonds, which are not specifically mentioned in the general formula (I). The possible stereoisomers, which are defined by their specific spatial form, such as enantiomers, diastereomers, Z and E isomers, are all embraced by the formula (I) and can be obtained by customary methods from mixtures of the stereoisomers or else be prepared by stereoselective reactions in combination with the use of stereochemically pure starting materials.

A sub-group of stereoisomers or enantiomers are the compounds (I) which, with respect to the asymmetrically substituted carbon atom to which the group R² is attached in formula (I), are present as pure stereoisomer (of the R or S configuration) or as a mixture of the R and S stereoisomers with an excess of one of the stereoisomers, preferably a content of R or S isomer of from 60 to 100%, in particular from. 70 to 100%, very particularly from 80 to 100%, very preferably from 90 to 100%, based on the total amount of the R and S isomers.

If the stereoisomers do not contain any other centers of asymmetry in the molecule in question, for example in the radicals R¹, R², R³, R⁴ and in the radicals X, the compounds of the formula (I) are pure enantiomers or enantiomer mixtures in which one of the enantiomers is present in excess, for example quantifiable by a specific rotation of polarized light (optical rotatory dispersion) which is different from zero or by other methods for determining the enantiomeric excess (ee %=enantiomeric excess in percent), for example by chromatography on chiral separation materials (cf. standard textbooks on stereochemistry).

Compounds (I) which are essentially enantiomerically pure can be obtained by enantioselective syntheses or else by optical resolution by customary methods, for example by crystallization or chiral chromatography.

In particular for reasons of better herbicidal activity, better selectivity and/or easier preparation, those novel compounds of the formula (I) mentioned or their salts are of particular interest in which individual radicals have one of the preferred meanings already mentioned or mentioned hereinbelow, or, in particular, those, in which one or more of the preferred meanings already mentioned or mentioned hereinbelow are combined.

R¹ is preferably phenyl which is unsubstituted or substituted by one or more radicals selected from the group consisting of halogen, hydroxyl, amino, nitro, formyl, carboxyl, sulfo, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylamino-carbonyl, di(C₁-C₄)alkylaminocarbonyl, (C₁-C₄)alkylsulfonyl and (C₁-C₄)haloalkylsulfonyl and, including substituents, has 6 to 30 carbon atoms, preferably 6 to 20 carbon atoms, in particular 6 to 15 carbon atoms.

R¹ is also preferably (C₃-C₉)cycloalkyl which is unsubstituted or substituted by one or more radicals selected from the group consisting of halogen, hydroxyl, amino, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino and di(C₁-C₄)alkylamino and, including substituents, has 3 to 30 carbon atoms, preferably 3 to 20 carbon atoms, in particular 3 to 15 carbon atoms.

R¹ is also preferably heterocyclyl which is unsubstituted or substituted by one or more radicals selected from the group consisting of halogen, hydroxyl, amino, nitro, formyl, carboxyl, sulfonyl, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl and (C₁-C₄)haloalkylsulfonyl and, including substituents, has 2 to 30 carbon atoms, preferably 2 to 20 carbon atoms, in particular 2 to 15 carbon atoms.

Here and also in other radicals, heterocyclyl is preferably a heterocyclic radical having 3 to 7, in particular 3 to 6, ring atoms and one heteroatom selected from the group consisting of N, O and S, for example pyridyl, thienyl, furyl, pyrrolyl, oxiranyl, oxetanyl, oxolanyl (=tetrahydrofuryl), oxanyl, pyrrolidyl or piperidyl or else pyrrolinyl or is a heterocyclic radical having two or three heteroatoms selected from the group consisting of pyrimidinyl, pyridazinyl, pyrazinyl, triazinyl, thienyl, thiazolyl, thiadiazolyl, oxazolyl, isoxazolyl, pyrazolyl, triazolyl, piperazinyl, dioxolanyl, oxazolinyl, isoxazolinyl, oxazolidinyl, isoxazolidinyl or morpholinyl.

R¹ is also preferably (C₁-C₆)alkyl, (C₂-C₆)alkenyl or (C₂-C₆)alkynyl, where each of the 3 lastmentioned radicals is unsubstituted or substituted by one or more radicals from the group consisting of halogen, hydroxyl, cyano, nitro, thiocyanato, (C₁-C₄)alkoxy, (C₁-C₄)haloalkoxy, (C₂-C₄)alkenyloxy, (C₂-C₄)haloalkenyloxy, (C₁-C₄)alkylthio, (C₁-C₄)alkylsulfinyl, (C₁-C₄)alkylsulfonyl, (C₁-C₄)haloalkylsulfinyl, (C₁-C₄)haloalkylsulfonyl and (C₃-C₆)cycloalkyl, which is unsubstituted or substituted by one or more radicals selected from the group consisting of halogen, hydroxyl, amino, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)halo-alkylthio, mono(C₁-C₄)alkylamino and di(C₁-C₄)alkylamino, and phenyl or heterocyclyl, where each of the two lastmentioned radicals is unsubstituted or substituted by one or more radicals selected from the group consisting of halogen, hydroxyl, amino, nitro, formyl, carboxyl, sulfonyl, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl and (C₁-C₄)halo-alkylsulfonyl, and radicals of the formulae R′—C(═Z′)—, R′—C(═Z′)—Z—, R′—Z—C(═Z′)—, R′R″N—C(═Z—)—, R′—Z—C(═Z′)—O—, R′R″N—C(═Z′)—Z—, R′—Z—C(═Z′)—NR″— and R′R″N—C(═Z′)—NR′″— in which R′, R″ and R′″ independently of one another are each (C₁-C₄)alkyl, phenyl, phenyl-(C₁-C₄)alkyl, (C₃-C₆)cycloalkyl or (C₃-C₆)cycloalkyl-(C₁-C₄)alkyl, where each of the 5 lastmentioned radicals is unsubstituted or substituted by one or more radicals selected from the group consisting of halogen, hydroxyl, amino, nitro, formyl, cyano, thiocyanato, (C₁-C₄)alkoxy, (C₁-C₄)alkylthio, mono(C₁-C₄)alkylamino, di(C₁-C₄)alkylamino, (C₂-C₄)alkenyl, (C₂C₄)alkynyl, (C₃-C₆)cycloalkyl and, in the case of cyclic radicals, also by C₁-C₄)alkyl and (C₁-C₄)haloalkyl and in which Z and Z′ independently of one another are each an oxygen or a sulfur atom,

and, including substituents, preferably has 1 to 20 carbon atoms, in particular 1 to 15 carbon atoms.

R¹ is in particular (C₁-C₄)alkyl which is unsubstituted or substituted by one or more radicals selected from the group consisting of halogen, (C₁-C₄)alkoxy, (C₁-C₄)alkylthio, (C₁-C₄)alkylsulfonyl, (C₃-C₉)cycloalkyl, which is unsubstituted or substituted as defined above or prefarably by one or more redicals selected from the group consisting of halogen, cyceno, (C₁-C₄)alkyl and (C₁-C₄)alkoxy, and phenyl, which is unsubstituted or substituted by one or more radicals selected from the group consisting of halogen, (C₁-C₄)alkyl, (C₁-C₄)haloalkyl, (C₁-C₄)alkoxy, (C₁-C₄)haloalkoxy, (C₁-C₄)alkylthio, amino, mono- and di[(C₁-C₄)alkyl]amino, (C₁-C₄)alkanoylamino, benzoylamino, nitro, cyano, [(C₁-C₄)alkyl]carbonyl, formyl, carbamoyl, mono- and di-[(C₁-C₄)alkyl]aminocarbonyl and (C₁-C₄)alkylsulfonyl, and heterocyclyl having 3 to 6 ring atoms and 1 to 3 hetero ring atoms selected from the group consisting of N, O and S, where the ring is unsubstituted or substituted by one or more radicals selected from the group consisting of halogen, (C₁-C₄)alkyl and oxo,

or is phenyl which is unsubstituted or substituted by one or more radicals selected from the group consisting of halogen, hydroxyl, amino, nitro, formyl, carboxyl, sulfonyl, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylamino-carbonyl, di(C₁-C₄)alkylaminocarbonyl, (C₁-C₄)alkylsulfonyl and (C₁-C₄)haloalkylsulfonyl and, including substituents, has 2 to 30 carbon atoms, preferably 2 to 20 carbon atoms, in particular 2 to. 15 carbon atoms.

R¹ is furthermore preferably (C₁-C₄)alkyl, (C₁-C₄)haloalkyl, benzyl or [(C₃-C₆)cycloalkyl]-(C₁-C₂)alkyl, in particular (C₁-C₄)alkyl, (C₁-C₄)haloalkyl or [(C₃-C₆)cycloalkyl]methyl, preferably —CH₃, —CH₂F, —CHF₂, —CF₃, —CH₂Cl, —CHCl₂, —CCl₃, —CH₂Br, —CHBr₂, —CH₂CH₃, —CH₂CH₂F, —CF₂CHF₂, —CH₂CH₂Cl, —CH₂CH₂Br —CH(CH₃)₂, —CF(CH₃)₂, —C(CH₃)₂Cl, —CH₂CH₂CH₂F, —CH₂CH₂CH₂Cl or cyclopropylmethyl.

Independently of the radicals R¹, R³, R⁴ and (X)ₙ and preferably in combination with preferred meanings of one or more of these radicals, the following meanings of R² are of particular interest:

R² is, for example, a cycloalkyl radical having 4 to 6 ring atoms, such as cyclobutyl, cyclopentyl or cyclohexyl, a bicyclic cyloalkyl radical having 4 to 6 ring atoms, a cycloalkenyl radical having 4 to 6 ring atoms, such as cyclobut-1-enyl, cyclobut-2enyl, cyclopent-1-enyl, cyclopent-2-enyl, cyclopent-3-enyl, cyclohex-1-enyl, cyclohex-2-enyl or cyclohex-3-enyl, or phenyl or a heterocyclic saturated or partially unsaturated radical having 3 to 6 ring atoms and one heteroatom selected from the group consisting of N, O and S, or a heteroaromatic radical having 5 or 6 ring atoms and one heteroatom selected from the group consisting of N, O and S or a heterocyclic radical having 2 to 4 heteroatoms selected from the group consisting of the heteroaromatic radicals pyrimidinyl, pyridazinyl, pyrazinyl, triazinyl, thiazolyl, thiadiazolyl, oxazolyl, isoxazolyl, pyrazolyl, imidazolyl, triazolyl and tetrazolyl or the partially unsaturated or saturated heterocyclic radicals piperazinyl, dioxolanyl, oxazolinyl, isoxazolinyl, oxazolidinyl, isoxazolidinyl and morpholinyl, where each of the cyclic radicals mentioned above for R² is unsubstituted or substituted and, including substituents, has preferably up to 30 carbon atoms, or is

a radical of the formula —A—Z, in which

A is straight-chain alkylene having 1 to 4 carbon atoms or straight-chain alkenylene or alkynylene having in each case 2 to 5 carbon atoms, where each of the three lastmentioned diradicals is unsubstituted or substituted by one or more radicals selected from the group consisting of halogen, nitro, cyano, thiocyanato, (C₁-C₄)alkyl, (C₁-C₄)alkoxy, (C₁-C₄)alkylthio, (C₁-C₄)haloalkyl and (C₁-C₄)haloalkoxy, and

Z is a cycloalkyl radical having 3 to 6 ring atoms, such as cyclopropyl, cyclobutyl, cyclopentyl or cyclohexyl, a bicyclic cycloalkyl radical having 4 to 6 ring atoms, a cycloalkenyl radical having 4 to 6 ring atoms, such as cyclobut-1-enyl, cyclobut-2-enyl, cyclopent-1-enyl, cyclopent-2-enyl, cyclopent-3-enyl, cyclohex-1-enyl, cyclohex-2-enyl oder cyclohex-3enyl, or is phenyl or a heterocyclic saturated or partially unsaturated radical having 3 to 6 ring atoms and one heteroatom selected from the group consisting of N, O and S, or is a heteroaromatic radical having 5 or 6 ring atoms and one heteroatom selected from the group consisting of N, O and S or is a heterocyclic radical having 2 to 4 heteroatoms selected from the group of the heteroaromatic radicals pyrimidinyl, pyridazinyl, pyrazinyl, triazinyl, thiazolyl, thiadiazolyl, oxazolyl, isoxazolyl, pyrazolyl, imidazolyl, triazolyl and tetrazolyl or the partially unsaturated or saturated heterocyclic radicals piperazinyl, dioxolanyl, oxazolinyl, isoxazolinyl, oxazolidinyl, isoxazolidinyl and morpholinyl, where each of the cyclic radicals mentioned for Z is unsubstituted or substituted and, including substituents, has preferably up to 30 carbon atoms.

A is here preferably a group of the formula —CH₂—, —CH₂CH₂—, —CH₂CH₂CH₂—or —CH₂CH₂CH₂CH₂, where each of the 4 lastmentioned diradicals is unsubstituted or substituted by one or more radicals selected from the group consisting of halogen, nitro, cyano, thiocyanato, (C₁-C₄)alkyl, (C₁-C₄)alkoxy, (C₁-C₄)alkylthio, (C₁-C₄)haloalkyl und (C₁-C₄)haloalkoxy.

R² is preferably cyclobutyl, cyclopentyl oder cyclohexyl, cyclobut-1-enyl, cyclobut-2-enyl, cyclopent-1-enyl, cyclopent-2-enyl, cyclopent-3-enyl, oxiranyl, oxetan-2-yl, oxetan-3-yl, oxolanyl (=tetrahydrofuryl), such as oxolan-2-yl or oxolan-3-yl, oxanyl, such as oxan-2-yl, oxan-3-yl or oxan4-yl, 1-, 2- or 3-azetidinyl, 1-, 2- or 3-azolidinyl (pyrrolidinyl), 1-, 2-, 3- or 4-piperidinyl, 2- or 3-furyl, 2- oder 3-thienyl, 2-, 3- or 4-pyridyl, 2- or 3-pyrrolyl or else pyrrolinyl or pyrimidinyl, pyridazinyl, pyrazinyl, triazinyl, thiazolyl, thiadiazolyl, oxazolyl, isoxazolyl, pyrazolyl, imidazolyl, triazolyl, tetrazolyl, piperazinyl, dioxolanyl, oxazolinyl, isoxazolinyl, oxazolidinyl, isoxazolidinyl oder morpholinyl, where each of the cyclic radicals mentioned above for R² is unsubstituted or substituted and, including substituents, has preferably up to 30 carbon atoms, or is a radical of the formula —A—Z, in which

A is straight-chain alkylene having 1 to 4 carbon atoms or straight-chain alkenylene or alkynylene having in each case 2 to 5 carbon atoms, where each of the three lastmentioned diradicals is unsubstituted or substituted by one or more radicals selected from the group consisting of halogen, nitro, cyano, thiocyanato, (C₁-C₄)alkyl, (C₁-C₄)alkoxy, (C₁-C₄)alkylthio, (C₁-C₄)haloalkyl and (C₁-C₄)haloalkoxy, and

Z is cyclopropyl, cyclobutyl, cyclopentyl or cyclohexyl, cyclobut-1-enyl, cyclobut-2-enyl, cyclopent-1-enyl, cyclopent-2-enyl, cyclopent-3-enyl, oxiranyl, oxetan-2-yl, oxetan-3-yl, oxolanyl (=tetrahydrofuryl), such as oxolan-2-yl or oxolan-3-yl, oxanyl, such as oxan-2-yl, oxan-3-yl or oxan4-yl, 1-, 2- or 3-azetidinyl, 1-, 2- or 3-azolidinyl (pyrrolidinyl), 1-, 2-, 3- or 4-piperidinyl, 2- or 3-furyl, 2- or 3-thienyl, 2-, 3- or 4-pyridyl, 2- or 3-pyrrolyl or else pyrrolinyl or pyrimidinyl, pyridazinyl, pyrazinyl, triazinyl, thiazolyl, thiadiazolyl, oxazolyl, isoxazolyl, pyrazolyl, imidazolyl, triazolyl, tetrazolyl, piperazinyl, dioxolanyl, oxazolinyl, isoxazolinyl, oxazolidinyl, isoxazolidinyl or morpholinyl, where each of the cyclic radicals mentioned above for Z is unsubstituted or substituted and, including substituents, has preferably up to 30 carbon atoms.

Preferred substituents at the cyclic radicals R² and Z are in each case one or more identical or different substituents selected from the group consisting of the radicals A), B), C) and D), where

group A) consists of the radicals halogen, hydroxyl, amino, nitro, formyl, carboxyl, aminocarbonyl, sulfo, cyano, thiocyanato and oxo,

group B) consists of the radicals (C₁-C₆)alkyl, (C₁-C₆)alkoxy, (C₁-C₆)alkylthio, mono(C₁-C₆)alkylamino, di(C₁-C₄)alkylamino, (C₂-C₆)alkenyl, (C₂-C₆)alkynyl, (C₃-C₉)cycloalkyl, (C₄-C₉)cycloalkenyl, (C₁-C₆)alkylidene, (C₄-C₉)cyclo-alkylidene, radicals of the formulae R^{a}—C(═Z*)—, R^{a}—C(═Z*)—Z**—, R^{a}Z**—C(═Z*)—, R^{a}R^{b}N—C(═Z*)—, R^{a}—Z**—C(═Z*)—O—, R^{a}R^{b}N—C(═Z*)—Z**—, R^{a}—Z**—C(═Z*)—NR^{b}— and R^{a}R^{b}N—C(═Z*)—NR^{c}—, in which R^{a}, R^{b} and R^{c} independently of one another are each (C₁-C₆)alkyl, phenyl, phenyl-(C₁-C₆)alkyl, (C₃-C₉)cycloalkyl or (C₃-C₉)cycloalkyl-(C₁-C₆)alkyl and in which Z* and Z** independently of one another are each an oxygen or sulfur atom,

group C) consists of the radicals according to group B), but each radical is substituted by one or more radicals selected from the group consisting of halogen, hydroxyl, amino, nitro, formyl, carboxyl, sulfo, cyano, thiocyanato, (C₁-C₄)alkoxy, (C₁-C₄)haloalkoxy, (C₁-C₄)alkylthio, (C₁-₄)haloalkylthio, mono(C₁-C₄)alkylamino, di(C₁-C₄)alkylamino, (C₃-C₉)-cycloalkyl, (C₄-C₉)cycloalkylene, (C₄-C₉)cycloalkylidene, [(C₁-C₄)alkyl]carbonyl, [(C₁-C₄)alkoxy]carbonyl, amino-carbonyl, mono(C₁-C₄)alkylaminocarbonyl, di(C₁-C₄)-alkylaminocarbonyl, phenyl, phenoxy, phenylthio, phenylcarbonyl, heterocyclyl, heterocyclyloxy, heterocyclylthio and heterocyclylamino,

where each of the 21 lastmentioned radicals is unsubstituted or substituted by one or more radicals selected from the group consisting of halogen, nitro, cyano, (C₁-C₄)alkoxy, (C₁-C₄)alkylthio, (C₁-C₄)halo-alkoxy, formyl, (C₁-C₄)alkylcarbonyl and (C₁-C₄)alkoxy-carbonyl and, in the case of cyclic radicals, also by (C₁-C₄)alkyl, (C₁-C₄)haloalkyl and (C₁-C₆)alkylidene and, in the case of cyclic radicals, also by (C₁-C₆)alkyl, (C₁-C₆)haloalkyl and (C₁-C₆)alkylidene, and

group D) consists of divalent or trivalent aliphatic bridges having 1 to 6, preferably 1 to 4 carbon atoms which, in the case of divalent bridges, connects two or, in the case of trivalent bridges, three carbon atoms of the cyclic skeleton, and the radical R² or Z thus being the radical of a bicycle or tricycle, where each of the bridges is unsubstituted or substituted by one or more substituents selected from the group consisting of halogen, nitro, cyano, (C₁-C₄)alkyl, (C₁-C₄)alkoxy, (C₁-C₄)alkylthio, (C₁-C₄)haloalkyl, (C₁-C₄)haloalkoxy, formyl, (C₁-C₄)alkyl-carbonyl, (C₁-C₄)alkoxycarbonyl and oxo,

and where R², including substituents, has preferably 3 to 20 carbon atoms, in particular 3 to 15 carbon atoms, and Z, including substituents, has preferably 4 to 20 carbon atoms, in particular 4 to 15 carbon atoms. Preferred (C₃-C₉)cycloalkyl radicals are cyclopropyl, cyclobutyl, cyclopentyl or cyclohexyl, in particular cyclopropyl, cyclobutyl or cyclopentyl.

The cyclic radicals R² and Z are preferably unsubstituted or substituted by preferred substituents. Preferred substituents for the cyclic radicals in R² and Z are in each case one or more identical or different substituents selected from the group consisting of halogen, hydroxyl, amino, nitro, formyl, carboxyl, aminocarbonyl, sulfo, cyano, thiocyanato, oxo, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₂-C₄)alkenyl, (C₂-C₄)alkynyl, (C₁-C₄)alkylidene, radicals of the formulae R^{a}—C(═Z*)—, R^{a}—C(═Z*)—Z**—, R^{a}—Z**—C(═Z*)—, R^{a}R^{b}N—C(═Z*)—, R^{a}—Z**—(═Z*)—O—, R^{a}R^{b}N—C(═Z*)—Z**—, R^{a}—Z**—C(═Z*)—NR^{b}— and R^{a}R^{b}N—C(═Z*)—NR^{c}—, in which R^{a}, R^{b} and R^{c} independently of one another are each (C₁-C₄)alkyl and in which Z* and Z** independently of one another are in each case an oxygen or sulfur atom, preferably an oxygen atom.

The cyclic R² or Z radicals are, in particular, unsubstituted or substituted by one or more radicals selected from the group consisting of halogen, hydroxyl, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, (C₁-C₄)alkylidene, mono(C₁-C₄)alkylamino, di(C₁-C₄)alkylamino, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl and (C₁-C₄)haloalkylsulfonyl.

Preferred compounds of the formula (I) according to the invention include those in which

R³ is hydrogen, (C₁-C₄)alkyl, which is unsubstituted or substituted by one or more radicals selected from the group consisting of halogen, hydroxyl, amino, cyano, thiocyanato, (C₁-C₄)alkoxy, (C₁-C₄)halo-alkoxy, (C₁-C₄)alkylthio, (C₁-C₄)haloalkylthio, mono(C₁-C₄)alkylamino and di(C₁-C₄)alkylamino, or is phenyl or (C₃-C₆)cycloalkyl, where each of the 2 lastmentioned radicals is unsubstituted or substituted by one or more radicals selected from the group consisting of halogen, hydroxyl, amino, nitro, formyl, carboxyl, sulfonyl, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl and (C₁-C₄)haloalkylsulfonyl, or

a radical of the formula N(B¹—D¹)(B²—D²), where B¹, B², D¹ and D² are as already defined or preferably as defined further below, in particular amino.

Preferred compounds of the formula (I) according to the invention include those in which

R⁴ is hydrogen, (C₁-C₄)alkyl, phenyl or (C₃-C₆)cycloalkyl, where each of the 3 lastmentioned radicals is unsubstituted or substituted by one or more radicals selected from the group consisting of halogen, hydroxyl, amino, nitro, formyl, carboxyl, sulfonyl, cyano, thiocyanato, (C₁-C₄)alkoxy, (C₁-C₄)haloalkoxy, (C₁-C₄)alkylthio, (C₁-C₄)halo-alkylthio, mono(C₁-C₄)alkylamino, di(C₁-C₄)alkylamino, (C₃-C₉)cyclo-alkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl, (C₁-C₄)haloalkylsulfonyl and, in the case of cyclic radicals, also by (C₁-C₄)alkyl and (C₁-C₄)haloalkyl, or is formyl, [(C₁-C₄)alkyl]carbonyl, [(C₁-C₄)alkoxy]carbonyl, aminocarbonyl, mono(C₁-C₄)alkylaminocarbonyl or di(C₁-C₄)alkylaminocarbonyl; in particular hydrogen, methyl, ethyl, n-propyl or isopropyl; very particularly preferably hydrogen.

Preferred compounds of the formula (I) according to the invention include those in which

B¹, B² and B³ independently of one another are each a direct bond or a divalent group of the formulae —C(═Z*)—, —C(═Z*)—Z**—, —C(═Z*)—NH— or —C(═Z*)—NR*—, where Z*=O or S, Z**=O or S and R*=(C₁-C₄)alkyl, phenyl, phenyl-(C₁-C₄)alkyl, (C₃-C₆)cycloalkyl or (C₃-C₆)cycloalkyl-(C₁-C₄)alkyl, where each of the 5 lastmentioned radicals is unsubstituted or substituted by one or more radicals selected from the group consisting of halogen, hydroxyl, amino, nitro, formyl, carboxyl, sulfo, cyano, thiocyanato, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl, (C₁-C₄)haloalkylsulfonyl and, in the case of cyclic radicals, also by (C₁-C₄)alkyl and (C₁-C₄)haloalkyl.

Furthermore preferably, B¹, B² and B³ independently of one another are a direct bond or a divalent group of the formula —C(═Z*)—, —C(═Z*)—Z**—, —C(═Z*)—NH— or —C(═Z*)—NR*—, where Z*=O or S, Z*=O or S and R*=(C₁-C₄)alkyl, phenyl, phenyl-(C₁-C₄)alkyl, (C₃-C₆)cycloalkyl or (C₃-C₆)cycloalkyl-(C₁-C₄)alkyl, where each of the 5 lastmentioned radicals is unsubstituted or substituted by one or more radicals selected from the group consisting of halogen, hydroxyl, amino, formyl, (C₁-C₄)alkoxy, (C₁-C₄)haloalkoxy, (C₁-C₄)alkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl and, in the case of cyclic radicals, also by (C₁-C₄)alkyl and (C₁-C₄)haloalkyl, in particular R*=(C₁-C₄)alkyl or (C₃-C₆)cycloalkyl or in particular R*=phenyl or phenyl-(C₁-C₄)alkyl, where each of the two lastmentioned radicals is unsubstituted in the phenyl moiety or substituted by one or more radicals selected from the group consisting of halogen, (C₁-C₄)alkyl, (C₁-C₄)haloalkyl, (C₁-C₄)alkoxy and (C₁-C₄)haloalkoxy.

Preferred compounds of the formula (I) according to the invention include those in which

D¹, D² and D³ in each case independently of one another hydrogen, (C₁-C₆)alkyl, phenyl, phenyl-(C₁-C₄)alkyl, (C₃-C₆)cycloalkyl or (C₃-C₆)-cycloalkyl-(C₁-C₆)alkyl, where each of the 5 lastmentioned radicals is unsubstituted or substituted, preferably unsubstituted or substituted by one or more radicals selected from the group consisting of halogen, hydroxyl, amino, nitro, formyl, carboxyl, sulfo, cyano, thiocyanato, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl, (C₁-C₄)haloalkylsulfonyl and, in the case of cyclic radicals, also by (C₁-C₄)alkyl and (C₁-C₄)haloalkyl.

Furthermore preferably, D¹, D² and D³ independently of one another are (C₁-C₄)alkyl, phenyl, phenyl-(C₁-C₄)alkyl, (C₃-C₆)cycloalkyl or (C₃-C₆)cycloalkyl-(C₁-C₄)alkyl, where each of the 5 lastmentioned radicals is unsubstituted or substituted by one or more radicals selected from the group consisting of halogen, hydroxyl, amino, formyl, (C₁-C₄)alkoxy, (C₁-C₄)haloalkoxy, (C₁-C₄)alkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl and, in the case of cyclic radicals, also by (C₁-C₄)alkyl and (C₁-C₄)haloalkyl, and are in particular (C₁-C₄)alkyl or (C₃-C₆)cycloalkyl or phenyl or phenyl-(C₁-C₄)alkyl, where each of the two lastmentioned radicals is unsubstituted in the phenyl moiety or substituted by one or more radicals selected from the group consisting of halogen, (C₁-C₄)alkyl, (C₁-C₄)haloalkyl, (C₁-C₄)alkoxy or (C₁-C₄)haloalkoxy.

Independently of the radicals R¹ to R⁴ and preferably in combination with preferred meanings of one or more of these radicals, the following meanings of (X)ₙ are of particular interest:

(X)ₙ are n substituents X, and X, independently of the others, is in each case preferably halogen, hydroxyl, amino, nitro, formyl, carboxyl, cyano, thiocyanato, aminocarbonyl or (C₁-C₄)alkyl, (C₁-C₄)alkoxy, (C₁-C₄)alkylthio, mono(C₁-C₄)alkylamino, di(C₁-C₄)alkylamino, (C₂-C₄)alkenyl, (C₂-C₄)alkynyl, [(C₁-C₄)alkyl]carbonyl, [(C₁-C₄)alkoxy]carbonyl, mono(C₁-C₄)alkylaminocarbonyl, di(C₁-C₄)alkyl-aminocarbonyl, N-(C₁-C₆)alkanoylamino or N-(C₁-C₄)alkanoyl-N-(C₁-C₄)alkylamino,

where each of the 13 lastmentioned radicals is unsubstituted or substituted, preferably unsubstituted or substituted by one or more radicals selected from the group consisting of halogen, hydroxyl, amino, cyano, thiocyanato, (C₁-C₄)alkoxy, (C₁-C₄)haloalkoxy, (C₁-C₄)alkylthio, mono(C₁-C₄)alkylamino, di(C₁-C₄)alkylamino, (C₃-C₆)cycloalkyl, (C₃-C₆)cyclo-alkylamino, [(C₁-C₄)alkyl]carbonyl, [(C₁-C₄)alkoxy]carbonyl, aminocarbonyl, mono(C₁-C₄)alkylaminocarbonyl, di(C₁-C₄)-alkylaminocarbonyl, phenyl, phenoxy, phenylthio, phenyl-carbonyl, heterocyclyl, heterocyclyloxy, heterocyclylthio and heterocyclylamino, where each of the 8 lastmentioned radicals is unsubstituted or substituted by one or more substituents selected from the group consisting of halogen, nitro, cyano, (C₁-C₄)alkyl, (C₁-C₄)alkoxy, (C₁-C₄)alkylthio, (C₁-C₄)haloalkyl, (C₁-C₄)haloalkoxy, formyl, (C₁-C₄)alkyl-carbonyl and (C₁-C₄)alkoxycarbonyl,

or is (C₃-C₉)cycloalkyl, phenyl, phenoxy, phenylthio, phenylcarbonyl, heterocyclyl, heterocyclyloxy, heterocyclylthio or heterocyclylamino,

where each of the 9 lastmentioned radicals is unsubstituted or substituted, preferably unsubstituted or substituted by one or more radicals selected from the group consisting of halogen, hydroxyl, amino, nitro, formyl, carboxyl, cyano, thiocyanato, (C₁-C₄)alkyl, (C₁-C₄)haloalkyl, (C₁-C₄)alkoxy, (C₁-C₄)halo-alkoxy, (C₁-C₄)alkylthio, (C₁-C₄)haloalkylthio, mono(C₁-C₄)-alkylamino, di(C₁-C₄)alkylamino, (C₃-C₆)cycloalkyl, [(C₁-C₄)alkyl]carbonyl, [(C₁-C₄)alkoxy]carbonyl, amino-carbonyl, mono(C₁-C₄)alkylaminocarbonyl and di(C₁-C₄)alkyl-aminocarbonyl,

or two adjacent radicals X together are a fused-on cycle having from 4 to 6 ring atoms which is carbocyclic or contains hetero ring atoms selected from the group consisting of O, S and N, and which is unsubstituted or substituted by one or more radicals selected from the group consisting of halogen, (C₁-C₄)alkyl and oxo;

n is preferably 0, 1, 2 or 3, in particular 1 or 2.

(X)ₙ are furthermore preferably n substituents X where X, independently of the others, is in each case halogen, hydroxyl, amino, nitro, formyl, carboxyl, cyano, thiocyanato, (C₁-C₄)alkyl, cyano-(C₁-C₄)alkyl, (C₁-C₄)alkoxy, (C₁-C₄)alkylamino, di[(C₁-C₄)alkyl]amino, halo-(C₁-C₄)alkyl, hydroxy-(C₁-C₄)alkyl, (C₁-C₄)alkoxy-(C₁-C₄)alkyl, halo(C₁-C₄)alkoxy-(C₁-C₄)alkyl, (C₁-C₄)alkylthio, halo(C₁-C₄)alkylthio, (C₂-C₆)alkenyl, halo(C₂-C₆)alkenyl, (C₂-C₆)alkynyl, halo(C₂-C₆)-alkynyl, (C₁-C₄)alkylamino-(C₁-C₄)alkyl, di[(C₁-C₄)alkyl]-amino-(C₁-C₄)alkyl, (C₃-C₆)cycloalkylamino-(C₁-C₄)alkyl, (C₃-C₉)cycloalkyl, heterocyclyl-(C₁-C₄)alkyl having 3 to 9 ring members, where the cyclic groups in the 3 lastmentioned radicals are unsubstituted or substituted by one or more radicals, preferably by up to three radicals, selected from the group consisting of (C₁-C₄)alkyl, halogen and cyano, or

is phenyl, phenoxy, phenylcarbonyl, phenylcarbonyl-(C₁-C₄)alkyl, (C₁-C₄)alkoxycarbonyl-(C₁-C₄)alkyl, (C₁-C₄)alkylaminocarbonyl-(C₁-C₄)alkyl, (C₁-C₄)alkylcarbonyl, (C₁-C₄)alkoxycarbonyl, aminocarbonyl, (C₁-C₄)alkylaminocarbonyl, phenoxy-(C₁-C₄)alkyl, phenyl-(C₁-C₄)alkyl, heterocyclyl, heterocyclylamino, heterocyclyloxy, heterocyclylthio or one of the 16 lastmentioned radicals which is substituted in the acyclic moiety or, preferably, in the cyclic moiety by one or more radicals selected from the group consisting of halogen, nitro, cyano, (C₁-C₄)alkyl, (C₁-C₄)alkoxy, (C₁-C₄)alkylthio, (C₁-C₄)haloalkyl, (C₁-C₄)haloalkoxy, formyl, (C₁-C₄)alkylcarbonyl, (C₁-C₄)alkoxycarbonyl, (C₁-C₄)alkoxy, where heterocyclyl in the radicals contains in each case 3 to 9 ring atoms and 1 to 3 hetero ring atoms selected from the group consisting of N, O and S, or

two adjacent radicals X together are a fused-on cycle having 4 to 6 ring atoms which is carbocyclic or contains hetero ring atoms selected from the group consisting of O, S and N and which is unsubstituted or substituted by one or more radicals selected from the group consisting of halogen, (C₁-C₄)alkyl and oxo.

(X)ₙ are particularly preferably n substituents X where X, independently of the others, is in each case halogen, OH, NO₂, CN, SCN, (C₁-C₆)alkyl, (C₁-C₆)alkoxy, (C₁-C₄)alkylcarbonyl or (C₁-C₄)alkyloxycarbonyl, where the four lastmentioned radicals are unsubstituted or substituted by halogen or (C₁-C₄)alkoxy, and are very particularly preferably n substitutents X where X, independently of the others, is in each case halogen, hydroxyl, (C₁-C₄)alkyl or (C₁-C₄)alkoxy.

The present invention also provides processes for preparing the compounds of the formula (I) or salts thereof, which comprise

a) reacting a compound of the formula (II)

R¹—Fu (II)

in which Fu is a functional group selected from the group consisting of the carboxylic esters, carboxylic orthoesters, carbonyl chlorides, carboxamides, carboxylic anhydrides and trichloromethyl with a group of the formula (III) or an acid addition salt thereof

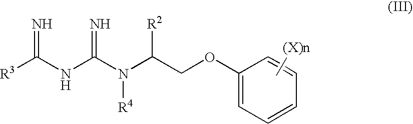

or

b) reacting a compound of the formula (IV),

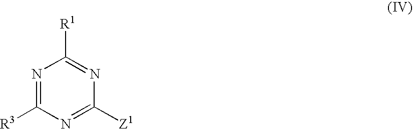

in which Z¹ is an exchangeable radical or a leaving group, for example chlorine, trichloromethyl, (C₁-C₄)alkylsulfonyl and unsubstituted or substituted phenyl-(C₁-C₄)alkylsulfonyl or (C₁-C₄)alkyl-phenylsulfonyl with a suitable amine of the formula (V) or an acid addition salt thereof

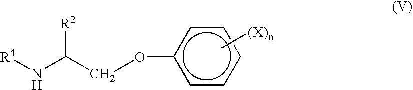

where in the formulae (II), (III), (IV) and (V) the radicals R¹, R², R³, R⁴, A¹, A² and X and also n are as defined in formula (I).

The compounds of the formula (II) and (III) are preferably reacted base-catalyzed in an inert organic solvent, such as, for example, tetrahydrofuran (THF), dioxane, acetonitrile, dimethylformamide (DMF), methanol and ethanol, at temperatures between −10° C. and the boiling point of the solvent, preferably at from 20° C. to 60° C.; if acid addition salts of the formula (III) are used, these are generally liberated in situ with the aid of a base. Suitable bases or basic catalysts are alkali metal hydroxides, alkali metal hydrides, alkali metal carbonates, alkali metal alkoxides, alkaline earth metal hydroxides, alkaline earth metal hydrides, alkaline earth metal carbonates or organic bases, such as triethylamine or 1,8-diazabicyclo-[5.4.0]undec-7-ene (DBU). The base in question is employed, for example, in the range from 0.1 to 3 molar equivalents, based on the compound of the formula (III). Based on the compound of the formula (III), the compound of the formula (II) can be employed, for example, in equimolar amounts or in an excess of up to 2 molar equivalents. In principle, the corresponding processes are known from the literature (compare: Comprehensive Heterocyclic Chemistry, A. R. Katritzky, C. W. Rees, Pergamon Press, Oxford, N.Y., 1984, Vol.3; Part 2B; ISBN 0-08-030703-5, p.290).

The compounds of the formulae (IV) and (V) are preferably reacted base-catalyzed in an inert organic solvent, such as, for example, THF, dioxane, acetonitrile, DMF, methanol and ethanol, at temperatures between −10° C. and the boiling point of the solvent or solvent mixture in question, preferably at from 20° C. to 60° C., where the compound (V), if it is employed as acid addition salt, is, if appropriate, liberated in situ using a base. Suitable bases or basic catalysts are alkali metal hydroxides, alkali metal hydrides, alkali metal carbonates, alkali metal alkoxides, alkaline earth metal hydroxides, alkaline earth metal hydrides, alkaline earth metal carbonates or organic bases such as triethylamine or 1,8-diaza-bicyclo[5.4.0]undec-7-ene (DBU). The base in question is generally employed in the range from 1 to 3 molar equivalents, based on the compound of the formula (IV), and the compound of the formula (IV) can be employed, for example, in equimolar amounts or in an excess of up to 2 molar equivalents, based on the compound of the formula (V). In principle, the corresponding processes are known from the literature (cf. Comprehensive Heterocyclic Chemistry, A. R. Katritzky, C. W. Rees, Pergamon Press, Oxford, N.Y., 1984, Vol.3; Part 2B; ISBN 0-08-030703-5, p. 482).

The starting materials of the formulae (II), (III), (IV) and (V) are either commercially available, or they can be prepared by or analogously to processes known from the literature. Some of the compounds of the formulae (III) and (V) are novel, and they also form part of the subject matter of the invention. The compounds can also be prepared, for example, by one of the processes described below.

In the case of biguanides (R³=unsubstituted or substituted amino), the optically active compounds of the formula (III) can be obtained, for example, by reacting optically active amines of the abovementioned formula (V) and cyanoguanidine of the formula H₂N—C(═NH)—NH—CN (see, for example, EP-A-492615). In general, the reaction can be carried out effectively under acid catalysis and in the presence of an organic solvent, such as an optionally halogenated hydrocarbon. Suitable catalysts are, for example, mineral acids, such as hydrogen chloride; suitable solvents are, for example, dichloromethane or n-decane. The reaction is carried out, for example, in the range from 0 to 200° C., preferably from 90 to 1800° C.

The optically active amines of the formula (V) required for the above reaction and the preparation variant b) are known or can be prepared by processes known per se (cf. Tetrahedron Lett. 29 (1988) 223-224, Tetrahedron Lett. 36 (1995) 3917-3920; Tetrahedron, Asymmetry 5 (1994) 817-820; EP-A-320898, EP-A443606, DE-A-3426919, DE-A4000610).

The compound of the formula (IV), or a direct precursor thereof, can be prepared, for example, as follows:

1. Reaction of a compound of the formula (II) with a thiourea derivative of the formula (VI),

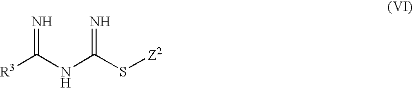

in which Z² is (C₁-C₄)alkyl or phenyl-(C₁-C₄)alkyl and R³ is as defined in formula (I) gives compounds of the formula (IV) in which Z₁=SZ² .

2. Reaction of an amidine of the formula (VII) or an acid addition salt thereof

H₂N—CR¹═NH (VII)

in which R¹ is as defined in formula (I)

with a N-cyanodithioiminocarbonate of the formula (VIII)

NC—N═C(S—Z³)₂ (VIII)

in which Z³ is (C₁-C₄)alkyl or phenyl-(C₁-C₄)alkyl gives compounds of the formula (IV) in which Z¹=—S—Z³ and R³=amino.

3. Reaction of an alkali metal dicyanamide with a carboxylic acid derivative of the formula (II) mentioned gives compounds of the formula (IV) in which Z¹=NH₂ and R³=amino.

4. Reaction of trichloroacetonitrile with a nitrile of the formula (IX)

R¹—CN (IX)

in which R¹ is as defined in formula (I) initially gives compounds of the formula (X)

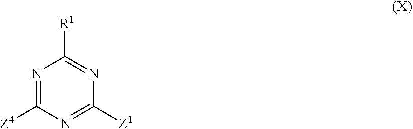

in which Z¹ and Z⁴ are each CCl₃ which, by subsequent reaction with compounds of the formula H—R³ (R³ is as defined in formula (I)), gives compounds of the formula (IV) in which Z¹=CCl₃.

The reaction of the carboxylic acid derivatives of the formula (II) with the thiourea derivatives of the formula (VI) is preferably carried out base-catalyzed in an organic solvent, such as, for example, acetone, THF, dioxane, acetonitrile, DMF, methanol, ethanol, at temperatures from −10° C. to the boiling point of the solvent, preferably at from −0° C. to 20° C. However, the reaction can also be carried out in water or in aqueous solvent mixtures with one or more of the abovementioned organic solvents. If (VI) is employed as acid addition salt, it can, if appropriate, be liberated in situ using a base. Suitable bases or basic catalysts are alkali metal hydroxides, alkali metal hydrides, alkali metal carbonates, alkali metal alkoxides, alkaline earth metal hydroxides, alkaline earth metal hydrides, alkaline earth metal carbonates or organic bases, such as triethylamine or 1,8-diazabicyclo[5.4.0]undec-7-ene (DBU). The base in question is, for example, employed in the range from 1 to 3 molar equivalents, based on the compound of the formula (VI). Compounds of the formula (II) and (VI) can be employed, for example, in equimolar amounts, or with an excess of up to 2 molar equivalents of the compound of the formula (II). In principle, the corresponding processes are known from the literature (compare; H. Eilingsfeld, H. Scheuermann, Chem. Ber.; 1967, 100, 1874), the corresponding intermediates of the formula (IV) are novel.

The reaction of the amidines of the formula (VII) with the N-cyanodithioiminocarbonates of the formula (VIII) is preferably carried out base-catalyzed in an inert organic solvent, such as, for example, acetonitrile, DMF, dimethylacetamide (DMA), N-methylpyrrolidone (NMP), methanol and ethanol, at temperatures from −10° C. to the boiling point of the solvent, preferably at from 20° C. to 80° C. If (VII) is employed as acid addition salt, it can, if appropriate, be liberated in situ using a base. Suitable bases or basic catalysts are alkali metal hydroxides, alkali metal hydrides, alkali metal carbonates, alkali metal alkoxides, alkaline earth metal hydroxides, alkaline earth metal hydrides, alkaline earth metal carbonates or organic bases, such as triethylamine or 1,8-diazabicyclo[5.4.0]undec-7-ene (DBU). The base in question is employed, for example, in the range from 1 to 3 molar equivalents, based on the compound of the formula (VIII), and compounds of the formulae (VII) and (VII) can generally be employed in equimolar amounts or with an excess of two molar equivalents of the compound of the formula (II). In principle, the corresponding processes are known from the literature (compare: T. A. Riley, W. J. Henney, N. K. Dailey, B. E. Wilson, R. K. Robins; J. Heterocyclic Chem.; 1986, 23 (6), 1706-1714), the corresponding intermediates of the formula (IV) are novel.

Intermediates of the formula (X) where Z¹=chlorine can be prepared by reacting alkali metal dicyanamide with a carboxylic acid derivative of the formula (II), Fu then preferably being the functional group carbonyl chloride or carboxamide. The reaction of the reaction components is carried out, for example, acid-catalyzed in an inert organic solvent, such as, for example, toluene, chlorobenzene, chlorinated hydrocarbons, at temperatures between −10° C. and the boiling point of the solvent, preferably at from 20° C. to 80° C., where the intermediates formed can be chlorinated in situ using a suitable chlorinating agent, such as, for example, phosphorus oxychloride. Suitable acids are, for example, hydrohalic acids, such as HCl, or else Lewis acids, such as, for example, AlCl₃ or BF₃ (compare U.S. Pat. No. 5,095,113, DuPont).

Intermediates of the formula (X) where Z¹, Z⁴=trihalomethyl can be prepared by reacting the corresponding trihaloacetonitriles with a carbonitrile of the formula (IX). The reaction of the reaction components is carried out, for example, acid-catalyzed in an inert organic solvent, such as, for example, toluene, chlorobenzene, chlorinated hydrocarbons, at temperatures between −40° C. and the boiling point of the solvent, preferably at from −10° C. to 30° C. Suitable acids are, for example, hydrohalic acids, such as HCl, or else Lewis acids, such as, for example, AlCl₃ or BF₃ (cf. EP-A-130939, Ciba Geigy).

Intermediates of the formula (IV), in which Z¹=(C₁-C₄)alkylmercapto or unsubstituted phenyl-(C₁-C₄)alkylmercapto, can be converted in an inert organic solvent, such as, for example toluene, chlorobenzene, chlorinated hydrocarbons or others, at temperatures between −40° C. and the boiling point of the solvent, preferably at from 20° C. to 80° C., with a suitable chlorinating agent, such as, for example elemental chlorine or phosporus oxychloride, into more reactive chlorotriazines of the formula (IV), in which Z¹=Cl (cf. J. K. Chakrabarti, D. E. Tupper; Tetrahedron 1975, 31(16), 1879-1882).

Intermediates of the formula (IV), in which Z¹=(C₁-C₄)alkylmercapto or unsubstituted phenyl-(C₁-C₄)alkylmercapto can be converted in an inert organic solvent, such as, for example toluene, chlorobenzene, chlorinated hydrocarbons or others, at temperatures between −40° C. and the boiling point of the solvent, preferably at from 20° C. to 80° C., with a suitable chlorinating agent, such as, for example elemental chlorine or phosporus oxychloride, into more reactive chlorotriazines of the formula (IV), in which Z¹=Cl (cf. J. K. Chakrabarti, D. E. Tupper; Tetrahedron 1975, 31(16), 1879-1882).

Intermediates of the formula (IV), in which Z¹=(C₁-C₄)alkylmercapto or unsubstituted or substituted phenyl-(C₁-C₄)-alkylmercapto or (C₁-C₄)alkyl-phenylthio can be oxidized in a suitable solvent, such as, for example, chlorinated hydrocarbons, acetic acid, water, alcohols, acetone or mixtures thereof at temperatures between 0° C. and the boiling point of the solvent, preferably at from 20° C. to 80° C., with a suitable oxidizing agent, such as, for example, m-chloroperbenzoic acid, hydrogen peroxide, potassium peroxomonosulfate (compare: T. A. Riley, W. J. Henney, N. K. Dailey, B. E. Wilson, R. K. Robins; J. Heterocyclic Chem.; 1986, 23 (6), 1706-1714).

Also possible for preparing enantiomers of the compounds (I) are customary methods for optical resolutions (cf. Textbooks of Stereochemistry), for example following processes for separating mixtures into diastereomers, for example physical processes, such as crystallization, chromatographic processes, in particular column chromatography and high pressure liquid chromatography, distillation, if appropriate under reduced pressure, extraction and other processes, it is possible to separate the remaining mixtures of enantiomers, generally by chromatographic separation on chiral solid phases. Suitable for preparative amounts or on an industrial scale are processes such as the crystallization of diastereomeric salts which can be obtained from the compounds (I) using optically active acids and, if appropriate, provided that acidic groups are present, using optically active bases.

Optically active acids which are suitable for optical resolution by crystallization of diastereomeric salts are, for example, camphorsulfonic acid, camphoric acid, bromocamphorsulfonic acid, quinic acid, tartaric acid, dibenzoyltartaric acid and other analogous acids; suitable optically active bases are, for example, quinine, cinchonine, quinidine, brucine, 1-phenylethylamine and other analogous bases.

The crystallizations are then in most cases carried out in aqueous or aqueous-organic solvents, where the diastereomer which is less soluble precipitates first, if appropriate after seeding. One enantiomer of the compound of the formula (I) is then liberated from the precipitated salt, or the other is liberated from the crystals, by acidification or using base.

The following acids are suitable for preparing the acid addition salts of the compounds of the formula (I): hydrohalic acids, such as hydrochloric acid or hydrobromic acid, furthermore phosphoric acid, nitric acid, sulfuric acid, mono- or bifunctional carboxylic acids and hydroxycarboxylic acids, such as acetic acid, maleic acid, succinic acid, fumaric acid, tartaric acid, citric acid, salicylic acid, sorbic acid or lactic acid, and also sulfonic acids, such as p-toluenesulfonic acid and 1,5-naphthalenedisulfonic acid. The acid addition compounds of the formula (I) can be obtained in a simple manner by the customary methods for forming salts, for example by dissolving a compound of the formula (I) in a suitable organic solvent, such as, for example, methanol, acetone, methylene chloride or benzine, and adding the acid at temperatures from 0 to 100° C., and they can be isolated in the known manner, for example by filtration, and, if appropriate, purified by washing with an inert organic solvent.

The base addition salts of the compounds of. the formula (I) are preferably prepared in inert polar solvents, such as, for example, water, methanol or acetone, at temperatures from 0 to 100° C. Examples of bases which are suitable for the preparation of the salts according to the invention are alkali metal carbonates, such as potassium carbonate, alkali metal hydroxides and alkaline earth metal hydroxides, for example NaOH or KOH, alkali metal hydrides and alkaline earth metal hydrides, for example NaH, alkali metal alkoxides and alkaline earth metal alkoxides, for example sodium methoxide or potassium tert-butoxide, or ammonia, ethanolamine or a quarternary ammonium hydroxide of the formula [NRR′R″R′″]⁺ OH⁻.

Solvents referred to as “inert solvents” in the above process variants are to be understood as meaning in each case solvents which are inert under the reaction conditions in question, but which need not be inert under any reaction conditions.

A collection of compounds of the formula (I) which can be synthesized by the abovementioned process may also be prepared in a parallel manner where the process may be carried but manually, partially automated or fully automated. In this case, it is possible, for example, to automate the procedure of the reaction, the work-up or the purification of the products or of the intermediates. In total, this is to be understood as meaning a procedure as is described, for example, by S. H. DeWitt in “Annual Reports in Combinatorial Chemistry and Molecular Diversity: Automated Synthesis”, Volume 1, Verlag Escom, 1997, pages 69 to 77.

A number of commercially available apparatuses as they are offered by, for example, Stem Corporation, Woodrolfe Road, Tollesbury, Essex, CM9 8SE, England, H+P Labortechnik GmbH, Bruckmannring 28, 85764 Oberschleiβheim, Germany may be used for the parallel procedure of the reaction and work-up. For the parallel purification of compounds (I), or of intermediates obtained during the preparation, use may be made, inter alia, of chromatography apparatuses, for example those from ISCO, Inc., 4700 Superior Street, Lincoln, Nebr. 68504, USA. The apparatuses mentioned lead to a modular procedure in which the individual process steps are automated, but manual operations have to be performed between the process steps. This can be avoided by employing semi-integrated or fully integrated automation systems where the automation modules in question are operated by, for example, robots. Such automation systems can be obtained, for example, from Zymark Corporation, Zymark Center, Hopkinton, Mass. 01748, USA.

In addition to what has been described here, compounds (I) may be prepared in part or fully by solid-phase-supported methods. For this purpose, individual intermediate steps or all intermediate steps of the synthesis or of a synthesis adapted to suit the procedure in question are bound to a synthetic resin. Solid-phase-supported synthesis methods are described extensively in the specialist literature, for example Barry A. Bunin, in “The Combinatorial Index”, Verlag Academic Press, 1998.

The use of solid-phase-supported synthesis methods permits a series of protocols which are known from the literature and which, in turn, can be performed manually or in an automated manner. For example, the “tea-bag method” (Houghten, U.S. Pat. No. 4,631,211; Houghten et al., Proc. Natl. Acad. Sci, 1985, 82, 5131-5135), in which products from IRORI, 11149 North Torrey Pines Road, La Jolla, Calif. 92037, USA, are employed, may be partially automated. The automation of solid-phase-supported parallel synthesis is performed successfully, for example, by apparatuses from Argonaut Technologies, Inc., 887 Industrial Road, San Carlos, Calif. 94070, USA or MultiSynTech GmbH, Wullener Feld 4, 58454 Witten, Germany.

The preparation methods described here give compounds (I) in the form of collections of substances known as libraries. The present invention also relates to libraries of the compounds (I) which contain at least two compounds (I) and their intermediates.

The compounds of the formula (I) according to the invention and their salts, hereinbelow together referred to as compounds of the formula (I) (according to the invention), have excellent herbicidal activity against a broad spectrum of economically important monocotyledonous and dicotyledonous harmful plants. The active compounds also act efficiently on perennial weeds which produce shoots from rhizomes, root stocks or other perennial organs and which are difficult to control. In this context, it is generally immaterial whether the substances are applied pre-sowing, pre-emergence or post-emergence.

Specifically, examples may be mentioned of some representatives of the monocotyledonous and dicotyledonous weed flora which can be controlled by the compounds according to the invention, without these being a restriction to certain species.

Examples of weed species on which the active compounds act efficiently are, from amongst the monocotyledons, Agrostis, Alopecurus, Apera, Avena, Brachicaria, Bromus, Dactyloctenium, Digitaria, Echinochloa, Eleocharis, Eleusine, Festuca, Fimbristylis, Ischaemum, Lolium, Monochoria, Panicum, Paspalum, Phalaris, Phleum, Poa, Sagittaria, Scirpus, Setaria, Sphenoclea, and also Cyperus species from the annual sector and from amongst the perennial species such as Agropyron, Cynodon, Imperata and Sorghum, and also perennial Cyperus species.

In the case of the dicotyledonous weed species, the spectrum of action extends to species such as, for example, Galium, Viola, Veronica, Lamium, Stellaria, Amaranthus, Sinapis, lpomoea, Matricaria, Abutilon and Sida from amongst the annuals, and Convolvulus, Cirsium, Rumex and Artemisia in the case of the perennial weeds. Moreover, herbicidal activity is observed in connection with dicotyledonous weeds such as Ambrosia, Anthemis, Carduus, Centaurea, Chenopodium, Cirsium, Convolvulus, Datura, Emex, Galeopsis, Galinsoga, Lepidium, Lindernia, Papaver, Portlaca, Polygonum, Ranunculus, Rorippa, Rotala, Seneceio, Sesbania, Solanum, Sonchus, Taraxacum, Trifolium, Urtica and Xanthium.

The active ingredients according to the invention also effect outstanding control of harmful plants which occur under the specific conditions of rice growing such as, for example, Sagittaria, Alisma, Eleocharis, Scirpus and Cyperus.

If the compounds according to the invention are applied to the soil surface prior to germination, then the weed seedlings are either prevented completely from emerging, or the weeds grow until they have reached the cotyledon stage but then their growth stops, and, eventually, after three to four weeks have elapsed, they die completely.

If the active compounds are applied post-emergence to the green parts of the plants, growth also stops drastically a very short time after the treatment and the weed plants remain at the developmental stage of the point in time of application, or they die completely after a certain time, so that in this manner competition by the weeds, which is harmful to the crop plants, is eliminated at a very early point in time and in a sustained manner.

Although the compounds according to the invention have an excellent herbicidal activity against monocotyledonous and dicotyledonous weeds, crop plants of economically important crops, for example wheat, barley, rye, rice, corn, sugar beet, cotton and soya, are not damaged at all, or only to a negligible extent. For these reasons, the present compounds are highly suitable for selectively controlling undesired plant growth in plantings of agriculturally useful plants.

In addition, the substances according to the invention have outstanding growth-regulating properties in crop plants. They engage in the plant metabolism in a regulating manner and can thus be employed for the targeted control of plant constituents and for facilitating harvesting, such as for example by provoking desiccation and stunted growth. Furthermore, they are also suitable for generally regulating and inhibiting undesirable vegetative growth, without destroying the plants in the process. Inhibition of vegetative growth plays an important role in many monocotyledonous and dicotyledonous crops because lodging can be reduced hereby, or prevented completely.

Owing to their herbicidal and plant growth-regulatory properties, the active compounds can also be employed for controlling harmful plants in crops of known or still to be developed genetically engineered plants. The transgenic plants generally have particularly advantageous properties, for example resistance to certain pesticides, in particular certain herbicides, resistance to plant diseases or causative organisms of plant diseases, such as certain insects or microorganisms such as fungi, bacteria or viruses. Other particular properties relate, for example, to the quantity, quality, storage-stability, composition and to specific ingredients of the harvested product. Thus, transgenic plants having an increased starch content or a modified quality of the starch or those having a different fatty acid composition of the harvested product are known.

The use of the compounds of the formula (I) according to the invention or their salts in economically important transgenic crops of useful and ornamental plants, for example of cereal, such as wheat, barley, rye, oats, millet, rice, maniok and corn, or else in crops of sugar beet, cotton, soya, oilseed rape, potato, tomato, pea and other vegetable species is preferred. The compounds of the formula (I) can preferably be used as herbicides in crops of useful plants which are resistant or which have been made resistant by genetic engineering toward the phytotoxic effects of the herbicides.

Conventional ways of preparing novel plants which have modified properties compared to known plants comprise, for example, traditional breeding methods and the generation of mutants. Alternatively, novel plants having modified properties can be generated with the aid of genetic engineering methods (see, for example, EP-A 0 221 044, EP-A 0 131 624). For example, there have been described several cases of

genetically engineered changes in crop plants in order to modify the starch synthesized in the plants (for example WO 92/11376, WO 92/14827 and WO 91/19806),

transgenic crop plants which are resistant to certain herbicides of the glufosinate- (cf., for example, EP-A 0 242 236, EP-A 0 242 246) or glyphosate-type (WO 92/00377), or of the sulfonylurea-type (EP-A 0 257 993, U.S. Pat. No. 5,013,659),

transgenic crop plants, for example cotton, having the ability to produce Bacillus thuringiensis toxins (Bt toxins) which impart resistance to certain pests to the plants (EP-A 0 142 924, EP-A 0 193 259),

transgenic crop plants having a modified fatty acid composition (WO 91/13972).

Numerous molecular biological techniques which allow the preparation of novel transgenic plants having modified properties are known in principle; see, for example, Sambrook et al., 1989, Molecular Cloning, A Laboratory Manual, 2nd ed. Cold Spring Harbor Laboratory Press, Cold Spring Harbor, N.Y.; or Winnacker “Gene und Klone” [Genes and Clones], VCH Weinheim, 2nd edition 1996, or Christou, “Trends in Plant Science” 1 (1996) 423A31).

In order to carry out such genetic engineering manipulations, it is possible to introduce nucleic acid molecules into plasmids which allow a mutagenesis or a change in the sequence to occur by recombination of DNA sequences. Using the abovementioned standard processes it is possible, for example, to exchange bases, to remove partial sequences or to add natural or synthetic sequences. To link the DNA fragments with each other, it is possible to attach adaptors or linkers to the fragments.

Plant cells having a reduced activity of a gene product can be prepared, for example, by expressing at least one appropriate antisense-RNA, a sense-RNA to achieve a cosuppression effect, or by expressing at least one appropriately constructed ribozyme which specifically cleaves transcripts of the abovementioned gene product.

To this end, it is possible to employ both DNA molecules which comprise the entire coding sequence of a gene product including any flanking sequences that may be present, and DNA molecules which comprise only parts of the coding sequence, it being necessary for these parts to be long enough to cause an antisense effect in the cells. It is also possible to use DNA sequences which have a high degree of homology to the coding sequences of a gene product but which are not entirely identical.

When expressing nucleic acid molecules in plants, the synthesized protein can be localized in any desired compartment of the plant cell. However, to achieve localization in a certain compartment, it is, for example, possible to link the coding region with DNA sequences which ensure localization in a certain compartment. Such sequences are known to the person skilled in the art (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106).

The transgenic plant cells can be regenerated to whole plants using known techniques. The transgenic plants can in principle be plants of any desired plant species, i.e. both monocotyledonous and dicotyledonous plants.

In this manner, it is possible to obtain transgenic plants which have modified properties by overexpression, suppression or inhibition of homologous (=natural) genes or gene sequences or by expression of heterologous (=foreign) genes or gene sequences.

The compounds (I) according to the invention can preferably be used in transgenic crops which are resistant to herbicides selected from the group consisting of the sulfonylureas, glufosinate-ammonium or glyphosate-isopropylammonium and analogous active compounds.

When using the active compounds according to the invention in transgenic crops, in addition to the effects against harmful plants which can be observed in other crops, there are frequently effects which are specific for the application in the respective transgenic crop, for example a modified or specifically broadened spectrum of weeds which can be controlled, modified application rates which can be used for the application, preferably good combinability with the herbicides to which the transgenic crop is resistant, and an effect on the growth and the yield of the transgenic crop plants.

The invention therefore also provides for the use of the compounds (I) according to the invention as herbicides for controlling harmful plants in transgenic crop plants.

The compounds according to the invention can be applied in the customary formulations in the form of wettable powders, emulsifiable concentrates, sprayable solutions, dusts or granules. The invention therefore also provides herbicidal and plant growth-regulating compositions comprising compounds of the formula (I).

The compounds of the formula (I) can be formulated in various ways depending on the prevailing biological and/or chemico-physical parameters. Examples of suitable formulation options are: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), oil- or water-based dispersions, oil-miscible solutions, capsule suspensions (CS), dusts (DP), seed-dressing compositions, granules for broadcasting and soil application, granules (GR) in the form of microgranules, spray granules, coating granules and adsorption granules, water-dispersible granules (WG), water-soluble granules (SG), ULV formulations, microcapsules and waxes.

These individual formulation types are known in principle and are described, for example, in Winnacker-Küchler, “Chemische Technologie” [Chemical Technology], Volume 7, C. Hauser Verlag Munich, 4th edition 1986; Wade van Valkenburg, “Pesticide Formulations”, Marcel Dekker, N.Y., 1973; K. Martens, “Spray Drying” Handbook, 3rd ed. 1979, G. Goodwin Ltd. London.

The necessary formulation auxiliaries, such as inert materials, surfactants, solvents and other additives, are likewise known and are described, for example, in: Watkins, “Handbook of Insecticide Dust Diluents and Carriers”, 2nd ed., Darland Books, Caldwell N.J., H. v. Olphen, “Introduction to Clay Colloid Chemistry”; 2nd ed., J. Wiley & Sons, N.Y.; C. Marsden, “Solvents Guide”; 2nd ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schönfeldt, “Grenzflächenaktive Äthylenoxidaddukte” [Surface-active ethylene oxide adducts], Wiss. Verlagsgesell., Stuttgart 1976; Winnacker-Küchler, “Chemische Technologie” [Chemical Technology], Volume 7, C. Hauser Verlag Munich, 4th edition 1986.

Based on these formulations it is also possible to produce combinations with other pesticidally active substances, for example insecticides, acaricides, herbicides and fungicides, and also with safeners, fertilizers and/or growth regulators, for example in the form of a ready-mix or tank mix.

Wettable powders are preparations which are uniformly dispersible in water and which, in addition to the active compound and as well as a diluent or inert substance, also contain surfactants of ionic and/or nonionic type (wetting agents, dispersants), for example polyethoxylated alkyl phenols, polyethoxylated fatty alcohols, polyethoxylated fatty amines, fatty alcohol polyglycol ethersulfates, alkanesulfonates, alkylbenzenesulfonates, sodium ligninsulfonate, sodium 2,2′-dinaphthylmethane6,6′-disulfonate, sodium dibutylnaphthalenesulfonate or else sodium oleoylmethyltaurinate. To prepare the wettable powders, the herbicidally active compounds are finely ground, for example in customary apparatuses such as hammer mills, fan mills and air-jet mills, and are mixed simultaneously or subsequently with the formulation auxiliaries.

Emulsifiable concentrates are prepared by dissolving the active compound in an organic solvent, for example butanol, cyclohexanone, dimethyl-formamide, xylene or else relatively high-boiling aromatics or hydrocarbons or mixtures of the organic solvents, with the addition of one or more surfactants of ionic and/or nonionic type (emulsifiers). Examples of emulsifiers which can be used are calcium alkylarylsulfonates, such as Ca dodecylbenzenesulfonate, or nonionic emulsifiers, such as fatty acid polyglycol esters, alkylaryl polyglycol ethers, fatty alcohol polyglycol ethers, propylene oxide-ethylene oxide condensation products, alkyl polyethers, sorbitan esters, for example sorbitan fatty acid esters or polyoxyethylene sorbitan esters, for example polyoxyethylene sorbitan fatty acid esters.

Dusts are obtained by grinding the active compound with finely divided solid substances, for example talc, natural clays, such as kaolin, bentonite and pyrophyllite, or diatomaceous earth.

Suspension concentrates can be water- or oil-based. They can be prepared, for example, by wet milling using commercially customary bead mills, with or without the addition of surfactants as already mentioned above, for example, in the case of the other formulation types.

Emulsions, for example oil-in-water emulsions (EW), can be prepared for example by means of stirrers, colloid mills and/or static mixers using aqueous organic solvents and, if desired, surfactants as already mentioned above, for example, in the case of the other formulation types.

Granules can be prepared either by spraying the active compound onto adsorptive, granulated inert material or by applying active-compound concentrates to the surface of carriers such as sand, kaolinites or granulated inert material, by means of adhesive binders, for example polyvinyl alcohol, sodium polyacrylate or else mineral oils. Suitable active compounds can also be granulated in the manner which is customary for the preparation of fertilizer granules, if desired as a mixture with fertilizers.

Water-dispersible granules are generally prepared by the customary processes, such as spray-drying, fluidized-bed granulation, disk granulation, mixing using high-speed mixers, and extrusion without solid inert material. For the preparation of disk, fluidized-bed, extruder and spray granules, see for example processes in “Spray-Drying Handbook” 3rd ed. 1979, G. Goodwin Ltd., London; J. E. Browning, “Agglomeration”, Chemical and Engineering 1967, pages 147 ff; “Perry's Chemical Engineer's Handbook”, 5th ed., McGraw-Hill, New York 1973, pp. 8-57.

For further details on the formulation of crop protection products, see for example G. C. Klingman, “Weed Control as a Science”, John Wiley and Sons., Inc., New York, 1961, pages 81-96 and J. D. Freyer, S. A. Evans, “Weed Control Handbook”, 5th ed., Blackwell Scientific Publications, Oxford, 1968, pages 101-103.

The agrochemical formulations generally contain from 0.1 to 99% by weight, in particular from 0.1 to 95% by weight, of active compound of the formula (I).

In wettable powders the concentration of active compound is, for example, from about 10 to 90% by weight, the remainder to 100% by weight consisting of customary formulation constituents. In emulsifiable concentrates the concentration of active compound can be from about 1 to 90%, preferably from 5 to 80%, by weight. Formulations in the form of dusts contain from 1 to 30% by weight of active compound, preferably most commonly from 5 to 20% by weight of active compound, while sprayable solutions contain from about 0.05 to 80%, preferably from 2 to 50%, by weight of active compound. In the case of water-dispersible granules, the content of active compound depends partly on whether the active compound is in liquid or solid form and on the granulation auxiliaries, fillers, etc. that are used. In water-dispersible granules the content of active compound, for example, is between 1 and 95% by weight, preferably between 10 and 80% by weight.

In addition, said formulations of active compound may comprise the tackifiers, wetting agents, dispersants, emulsifiers, penetrants, preservatives, antifreeze agents, solvents, fillers, carriers, colorants, anti-foams, evaporation inhibitors and pH and viscosity regulators which are customary in each case.

The compounds of the formula (I) or their salts can be used as such or combined in the form of their preparations (formulations) with other pesticidally active compounds, such as, for example, insecticides, acaricides, nematicides, herbicides, fungicides, safeners, fertilizers and/or growth regulators for example as finished formulations or tank mix. Suitable active compounds which can be combined with the active compounds according to the invention in mixed formulations or in a tank mix are, for example, known active compounds, whose effect is based on an inhibition of, for example, acetolactate synthase, acetyl-CoA carboxylase, PS I, PS II, HPPDO, phytoene desaturase, protoporphyrinogen oxidase, glutamine synthetase, cellulose biosynthesis, 5-enolpyruvylshikimate-3-phosphate synthetase. Such compounds, and also other compounds that can be used, with a mechanism of action that is, in some cases, unknown or different, are described, for example, in Weed Research 26, 441-445 (1986), or in “The Pesticide Manual”, 11th edition 1997 (hereafter also abbreviated to “PM”) and 12^{th} edition 2000, The British Crop Protection Council and the Royal Soc. of Chemistry (publisher), and in the literature cited therein. For example, the following active compounds may be mentioned as herbicides which are known from the literature and which can be combined with the compounds of the formula (I) (note: the compounds are either referred to by the “common name” in accordance with the International Organization for Standardization (ISO) or by the chemical names, if appropriate together with a customary code number):

acetochlor; acifluorfen(-sodium); aclonifen; AKH 7088, i.e. [[[1-[5-[2-chloro-4-(trifluoromethyl)phenoxy]-2-nitrophenyl]-2-methoxyethylidene]amino]oxy]-acetic acid and its methyl ester; alachlor; alloxydim(-sodium); ametryn; amicarbazone; amidochlor, amidosulfuron; amitrol; AMS, i.e. ammonium sulfamate; anilofos; asulam; atrazine; azafenidin; azimsulfuron (DPX-A8947); aziprotryn; barban; BAS 516 H, i.e. 5-fluoro-2-phenyl-4H-3,1-benzoxazina-one; beflubutamide; benazolin(-ethyl); benfluralin; benfuresate; bensulfuron(-methyl); bensulide; bentazone; benzobicyclone; benzofenap; benzofluor; benzoylprop(-ethyl); benzthiazuron; bialaphos; bifenox; bispyribac(-sodium); bromacil; bromobutide; bromofenoxim; bromoxynil; bromuron; buminafos; busoxinone; butachlor; butafenacil; butamifos; butenachlor; buthidazole; butralin; butroxydim; butylate; cafenstrole (CH-900); carbetamide; cafentrazone(-ethyl); caloxydim; CDAA, i.e. 2-chloro-N,N-di-2-propenylacetamide; CDEC, i.e. 2-chloroallyl diethyldithiocarbamate; chlomethoxyfen; chloramben; chlorazifop-butyl; chlormesulon; chlorbromuron; chlorbufam; chlorfenac; chlorflurecol-methyl; chloridazon; chlorimuron(-ethyl); chlornitrofen; chlorotoluron; chloroxuron; chlorpropham; chlorsulfuron; chlorthal-dimethyl; chlorthiamid; chlortoluron, cinidon(-ethyl and -methyl); cinmethylin; cinosulfuron; clefoxydim; clethodim; clodinafop and its ester derivatives (for example clodinafop-propargyl); clomazone; clomeprop; cloproxydim; clopyralid; clopyrasulfuron(-methyl); cloransulam(-methyl); cumyluron (JC 940); cyanazine; cycloate; cyclosulfamuron (AC 104); cycloxydim; cycluron; cyhalofop and its ester derivatives (for example cyhalofop-butyl, DEH-112); cyperquat; cyprazine; cyprazole; daimuron; 2,4-D; 2,4-DB; dalapon; desmedipham; desmetryn; di-allate; dicamba; dichlobenil; dichlorprop; diclofop and its esters such as diclofop-methyl; diclosulam, diethatyl(-ethyl); difenoxuron; difenzoquat; diflufenican; diflufenzopyr; dimefuron; dimepiperate; dimethachlor; dimethametryn; dimethenamid (SAN-582H); dimethazone, dimexyflam, dimethipin; dimetrasulfuron, dinitramine; dinoseb; dinoterb; diphenamid; dipropetryn; diquat; dithiopyr; diuron; DNOC; eglinazine-ethyl; EL 77, i.e. 5-cyano-1-(1,1-dimethyl-ethyl)-N-methyl-1H-pyrazole4-carboxamide; endothal; EPTC; esprocarb; ethalfluralin; ethametsulfuron(-methyl); ethidimuron; ethiozin; ethofumesate; ethoxyfen and its esters (for example the ethyl ester, HN-252); ethoxysulfuron; etobenzanid (HW 52); F5231, i.e. N-[2-chloro-4-fluoro-5-[4-(3-fluoropropyl)4,5-dihydro-5-oxo-1H-tetrazol-1-yl]-phenyl]ethanesulfonamide; fenoprop; fenoxan, fenoxaprop and fenoxaprop-P and their esters, for example fenoxaprop-P-ethyl and fenoxaprop-ethyl; fenoxydim; fentrazamide; fenuron; flamprop(-methyl or -isopropyl or -isopropyl-L); flazasulfuron; floazulate, florasulam; fluazifop and fluazifop-P and their esters for example fluazifop-butyl and fluazifop-P-butyl; flucarbazone(-sodium); fluchloralin; flumetsulam; flumeturon; flumiclorac(-pentyl); flumioxazin (S482); flumipropyn; fluometuron; fluorochloridone, fluorodifen; fluoroglycofen(-ethyl); flupoxam (KNW-739); flupropacil (UBIC4243); flupyrsulfuron(-methyl, or -sodium); flurenol(-butyl); fluridone; flurochloridone; fluroxypyr(-meptyl); flurprimidol, flurtamone; fluthiacet(-methyl); fluthiamide; fomesafen; foramsulfuron; fosamine; furyloxyfen; glufosinate(-ammonium); glyphosate(-isopropylammonium); halosafen; halosulfuron(-methyl) and its esters (for example the methyl ester, NC-319); haloxyfop and its esters; haloxyfop-P (═R-haloxyfop) and its esters; hexazinone; imazamethabenz(-methyl); imazapyr; imazaquin and salts such as the ammonium salts; imazamethapyr; imazamox; imazapic imazethamethapyr; imazethapyr, imazosulfuron; indanofan; ioxynil; isocarbamid; isopropalin; isoproturon; isouron; isoxaben; isoxachlortole; isoxaflutole; isoxapyrifop; karbutilate; lactofen; lenacil; linuron; MCPA; MCPB; mecoprop; mefenacet; mefluidid; mesosulfuron, mesotrione; metamitron; metazachlor; methabenzthiazuron; metham; methazole; methoxyphenone; methyldymron; metabenzuron, methobenzuron; metobromuron; (alpha-)metolachlor; metosulam (XRD 511); metoxuron; metribuzin; metsulfuron-methyl; MH; molinate; monalide; monocarbamide dihydrogensulfate; monolinuron; monuron; MT 128, i.e. 6chloro-N-(3-chloro-2-propenyl)-5-methyl-N-phenyl-3-pyridazinamine; MT 5950, i.e. N-[3-chloro4-(1-methylethyl)-phenyl]-2-methylpentanamide; naproanilide; napropamide; naptalam; NC 310, i.e. 4-(2,4-dichlorobenzoyl)-1-methyl-5-benzyloxypyrazole; neburon; nicosulfuron; nipyraclophen; nitralin; nitrofen; nitrofluorfen; norflurazon; orbencarb; oryzalin; oxadiargyl (RP-020630); oxadiazone; oxasulfuron; oxaciclomefone; oxyfluorfen; paraquat; pebulate; pelargonic acid; pendimethalin; pentoxazone; perfluidone; phenisopham; phenmedipham; picloram; picolinafen; piperophos; piributicarb; pirifenop-butyl; pretilachior; primisulfuron(-methyl); procarbazone(-sodium); procyazine; prodiamine; profluralin; proglinazine(-ethyl); prometon; prometryn; propachlor; propanil; propaquizafop and its esters; propazine; propham; propisochlor; propyzamide; prosulfalin; prosulfocarb; prosulfuron (CGA-152005); prynachlor; pyroflufen(-ethyl); pyrazolinate; pyrazon; pyrazosulfuron(-ethyl); pyrazoxyfen; pyribenzoxim; pyributicarb; pyridafol; pyridate; pyriminobac(-methyl); pyrithiobac(-sodium) (KIH-2031); pyroxofop and its esters (for example propargyl ester); quinclorac; quinmerac; quinoclamine, quinofop and its ester derivatives, quizalofop and quizalofop-P and their ester derivatives, for example quizalofop-ethyl; quizalofop-P-tefuryl and -ethyl; renriduron; rimsulfuron (DPX-E 9636); S 275, i.e. 2-[4-chloro-2-fluoro-5-(2-propynyloxy)phenyl]4,5,6,7-tetrahydro-2H-indazole; secbumeton; sethoxydim; siduron; simazine; simetryn; SN 106279, i.e. 2-[[7-[2-chloro-4-(trifluoromethyl)phenoxy]-2-naphthalenyl]oxy]propanoic acid and its methyl ester; sulcotrione; sulfentrazone (FMC-97285, F-6285); sulfazurone; sulfometuron-(-methyl); sulfosate (ICI-A0224); sulfosulfuron; TCA; tebutam (GCP-5544); tebuthiuron; tepraloxydim; terbacil; terbucarb; terbuchlor; terbumeton; terbuthylazine; terbutryn; TFH 450, i.e. N,N-diethyl-3-[(2-ethyl-6-methylphenyl)sulfonyl]-1H-1,2,4-triazole-1-carboxamide; thenylchlor (NSK-850); thiafluamide; thiazafluron; thiazopyr (Mon-13200); thidiazimin (SN-24085); thifensulfuron(-methyl); thiobencarb; tiocarbazil; tralkoxydim; tri-allate; triasulfuron; triaziflam; triazofenamide; tribenuron(-methyl); triclopyr; tridiphane; trietazine; trifluralin; triflusulfuron and esters (e.g. the methyl estser, DPX66037); trimeturon; tritosulfuron; tsitodef; vernolate; WL 110547, i.e. 5-phenoxy-1-[3-(trifluoromethyl)phenyl]-1H-tetrazole; BAY MKH 6561, UBH-509; D489; LS 82-556; KPP-300; NC-324; NC-330; KH-218; DPX-N8189; SC-0774; DOWCO-535; DK-8910; V-53482; PP-600; MBH-001; KIH-9201; ET-751; KIH6127 and KIH-2023.

Of particular interest is the selective control of harmful plants in crops of useful and ornamental plants. Although the compounds (I) according to the invention have very good to satisfactory selectivity in a large number of crops, it is possible in principle that phytoxicity in the crop plants can occur in some crops and, in particular, when the compounds (I) are mixed with other herbicides which are less selective. In this respect, the combinations of the compounds (I) according to the invention which contain the compounds (I), or their combinations with other herbicides or pesticides, and safeners are of particular interest. The safeners, which are employed in such amounts that they act as antidotes, reduce the phytotoxic side effects of. the herbicides/pesticides used, for example in economically important crops such as cereals (wheat, barley, rye, maize, rice, millet), sugar beet, sugar cane, seed rape, cotton and soya, preferably cereal. Suitable safeners for the compounds (I) and their combinations with other pesticides are, for example, the following groups of compounds:

a) Compounds of the type of dichlorophenylpyrazoline-3-carboxylic acid, preferably compounds such as ethyl 1-(2,4-dichlorophenyl)-5-(ethoxycarbonyl)-5-methyl-2-pyrazoline-3-carboxylate (S1-1) (“mefenpyr-diethyl”, PM, pp. 781-782), and related compounds, as described in WO 91/07874,

b) Derivatives of dichlorophenylpyrazole carboxylic acid, preferably compounds such as ethyl 1-(2,4-dichlorophenyl)-5-methylpyrazole-3-carboxylate (S1-2), ethyl 1-(2,4-dichlorophenyl)-5-isopropyl-pyrazole-3-carboxylate (S1-3), ethyl 1-(2,4-dichlorophenyl)-5-(1,1-dimethylethyl)pyrazole-3-carboxylate (S1-4), ethyl 1-(2,4-dichlorophenyl)-5-phenylpyrazole-3-carboxylate (S1-5) and related compounds as described in EP-A-333 131 and EP-A-269 806.

c) Compounds of the type of the triazolecarboxylic acids (S1), preferably compounds such as fenchlorazole(ethyl ester), i.e. ethyl 1-(2,4-dichlorophenyl)-5-trichloromethyl-(1H)-1,2,4-triazole-3-carboxylate (S1) and related compounds as described in EP-A-174 562 and EP-A-346 620.

d) Compounds of the type of the 5-benzyl- or 5-phenyl-2-isoxazoline-3-carboxylic acid, or the 5,5diphenyl-2-isoxazoline-3carboxylic acid, preferably compounds such as ethyl 5-(2,4-dichlorobenzyl)-2isoxazoline-3-carboxylate (S1-7) or ethyl 5-phenyl-2-isoxazoline-3-carboxylate (S1-8) and related compounds, as described in WO 91/08202, or ethyl 5,5-diphenyl-2-isoxazoline-carboxylate (S1-9) (“isoxadifen-ethyl”) or its -n-propyl ester (S1-10) or ethyl 5-(4-fluorophenyl)-5-phenyl-2-isoxazoline-3-carboxylate (S1-11), as described in the German patent application (WO-A-95/07897).

e) Compounds of the type of the 8-quinolineoxyacetic acid (S2), preferably

1-methylhex-1-yl (5-chloro-8-quinolineoxy) acetate (common name “cloquintocet-mexyl” (S2-1) (see PM, pp. 263-264)

1,3-dimethylbut-1-yl (5-chloro-8-quinolineoxy)acetate (S2-2),

4-allyloxybutyl (5-chloro-8-quinolineoxy)acetate (S2-3),

1-allyloxyprop-2-yl (5-chloro-8-quinolineoxy)acetate (S24),

ethyl (5-chloro-8-quinolineoxy)acetate (S2-5),

methyl (5-chloro-8-quinolineoxy)acetate (S26),

allyl (5-chloro-8-quinolineoxy)acetate (S2-7),

2-(2-propylideneiminoxy)-1-ethyl (5-chloro-8-quinolineoxy)acetate (S2-8),

2-oxoprop-1-yl (5-chloro-8-quinolineoxy)acetate (S2-9)

and related compounds, as described in EP-A-86 750, EP-A-94 349 and EP-A-1 91 736 or EP-A-0 492 366.

f) Compounds of the type of the (5-chloro-8-quinolineoxy)malonic acid, preferably compounds such as diethyl (5-chloro-8-quinolineoxy)malonate, diallyl (5-chloro-8-quinolineoxy)malonate, methyl ethyl (5-chloro-8-quinolineoxy)malonate and related compounds, as described in EP-A-0 582 198.

g) Active comounds of the type of the phenoxyacetic or -propionic acid derivatives or the aromatic carboxylic acids, such as, for example, 2,4-dichlorophenoxyacetic acid (esters) (2,4-D), 4-chloro-2-methylphenoxypropionic esters (Mecoprop), MCPA or 3,6-dichloro-2-methoxybenzoic acid (esters) (Dicamba).

h) Active compounds of the type of the pyrimidines, which are used as soil-acting safeners in rice, such as, for example, “fenclorim” (PM, pp. 512-511) (=4,6-dichloro-2-phenylpyrimidine), which is known as safener for pretilachlor in sown rice,

i) Active compounds of the type of the dichloroacetamides, which are frequently used as pre-emergent safeners (soil-acting safeners), such as, for example,

“dichlormid” (PM, pp. 363-364) (=N,N-diallyl-2,2-dichloroacetamide),

“R-29148” (=3-dichloroacetyl-2,2,5-trimethyl-1,3-oxazolidine from Stauffer),

“benoxacor” (PM, pp. 102-103) (=4-dichloroacetyl-3,4-dihydro-3-methyl-2H-1,4-benzoxazine),

“PPG-1292” (=N-allyl-N-[(1,3-dioxolan-2-yl)methyl]dichloro-acetamide from PPG Industries),

“DK-24” (=N-allyl-N-[(allylaminocarbonyl)methyl]dichloroacetamide from Sagro-Chem),

“AD67” or “MON 4660” (=3-dichloroacetyl-1-oxa-3-aza-spiro[4,5]decane from Nitrokemia or Monsanto),

“diclonon” or “BAS145138” or “LAB145138” (=3-dichloroacetyl-2,5,5-trimethyl-1,3-diazabicyclo[4.3.0]nonane from BASF) and

“furilazol” or “MON 13900” (see PM, 637638) (=(RS)-3-dichloroacetyl-5-(2-furyl)-2,2-dimethyloxazolidine)

j) Active compounds of the type of the dichloroacetone derivatives, such as, for example,

“MG 191” (CAS-Reg. No. 96420-72-3) (=2-dichloromethyl-2-methyl-1,3-dioxolane from Nitrokemia), which is known as safener for maize,

k) Active compounds of the type of the oxyimino compounds, which are known as seed dressings, such as, for example,

“oxabetrinil” (PM, pp. 902-903) (=(Z)-1,3-dioxolan-2-ylmethoxy-imino(phenyl)acetonitrile), which is known as seed dressing safener for millet against metolachlor damage,

“fluxofenim” (PM, pp. 613614) (=1-(4-chlorophenyl)-2,2,2-trifluoro-1-ethanone O-(1,3-dioxolan-2-ylmethyl)oxime), which is known as seed dressing safener for millet against metolachlor damage,

“cyometrinil” or “-CGA43089” (PM, p. 1304) (=(Z)-cyanomethoxyimino(phenyl)acetonitrile), which is known as seed dressing safener for millet against metolachlor damage,

l) Active compounds of the type of the thiazolecarboxylic esters, which are known as seed dressings, such as, for example,

“flurazol” (PM, pp. 590-591) (=benzyl 2-chloro4-trifluoromethyl-1,3-thiazole-5-carboxylate), which is known as seed dressing safener for millet against alachlor and metolachlor damage,

m) Active compounds of the type of the naphthalenedicarboxylic acid derivatives, which are known as seed dressings, such as, for example,

“naphthalic anhydride” (PM, p. 1342) (=1,8-naphthalenedicarboxylic anhydride), which is known as seed dressing safener for maize against thiocarbamate herbicide damage,

n) Active compounds of the type of the chromanacetic acid derivatives, such as, for example,

“CL 304415” (CAS-Reg. No. 31541-57-8) (=2-(4-carboxychroman-4-yl)acetic acid from American Cyanamid), which is known as safener for maize against imidazolinone damage,

o) Active compounds which, in addition to a herbidical action against harmful plants, also have safener action in crop plants such as rice, such as, for example,

“dimepiperate” or “MY-93” (PM, pp. 404-405) (=S-1-methyl-1-phenylethyl piperidine-1-thiocarboxylate), which is known as safener for rice against herbicide molinate damage,

“daimuron” or “SK 23” (PM, p. 330) (=1-(1-methyl-1-phenylethyl)-3-p-tolylurea), which is known as safener for rice against herbicide imazosulfuron damage,

“cumyluron” =“JC-940” (=3-(2-chlorophenylmethyl)-1-(1-methyl-1-phenylethyl)urea, see JP-A60087254), which is known as safener for rice against damage by some herbicides,

“methoxyphenon” or “NK 049” (=3,3′-dimethyl-4-methoxy-benzophenone), which is known as safener for rice against damage by some herbicides,

“CSB” (=1-bromo4-(chloromethylsulfonyl)benzene) (CAS-Reg. No. 54091-06-4 from Kumiai), which is known as safener against damage by some herbicides in rice

p) N-Acylsulfonamides of the formula (S3) and salts thereof,

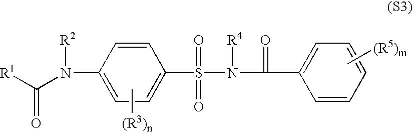

as described in WO-A-97/45016,

q) Acylsulfamoylbenzoamides of the formula (S4), if appropriate also in salt form,

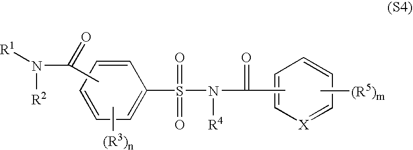

as described in the International Application No. PCT/EP98/06097, and

r) compounds of the formula (S5),

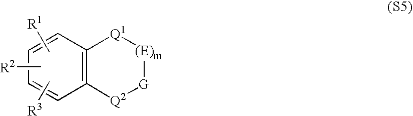

as described in WO-A 98/13 361,

including the stereoisomers and the salts used in agriculture.

Among the safeners mentioned, (S1-1) and (S1-9) and (S2-1), in particular (S1-1) and (S1-9) are of particular interest.

Some of the safeners are already known as herbicides and consequently show, in addition to the herbicidal action against harmful plants, also protective action in connection with crop plants.

The ratios by weight of herbicide (mixture) to safener generally depend on the application rate of the herbicide and the efficacy of the safener in question and can vary within wide limits, for example in the range from 200:1 to 1:200, preferably 100:1 to 1:100, in particular 20:1 to 1:20. Analogously to the compounds (I) or their mixtures, the safeners can be formulated with other herbicides/pesticides and be provided and used as ready mix or tank mix with the herbicides.

For use, the formulations which are present in commercially available form are, if appropriate, diluted in the customary manner, for example using water in the case of wettable powders, emulsifiable concentrates, dispersions and water-dispersible granules. Preparations in the form of dusts, granules for soil application or broadcasting and sprayable solutions are usually not further diluted with other inert substances prior to use.

The application rate of the compounds of the formula (I) required varies with the external conditions, such as temperature, humidity, the nature of the herbicide used and the like. It can vary within wide limits, for example between 0.001 and 10.0 kg/ha or more of active substance, but it is preferably between 0.005 and 5 kg/ha.

In the examples below, the amounts (including percentages) are based on weight, unless specifically defined otherwise.

### A. CHEMICAL EXAMPLES

### Example A1

### 2-Amino-4-(1,1,2,2-tetrafluoroethyl)6-{N-[1-cyclopentyl-2-(3-methylphenoxy)ethyl]amino}-1,3,5-triazine (See Table 1, Example 110)

A solution of 1.2 g (0.05 mol) of sodium in 50 ml of methanol was added to 6.7 g (0.02 mol) of 1-(2-biguanidino-2-cyclopentylethoxy)-3-methylbenzene in 50 ml of methanol. 7.2 g (0.045 mol) of methyl 1,1,2,2-tetrafluoropropanionate were then added, and the mixture was stirred under reflux for 4 h. The reaction mixture was concentrated and the residue was taken up in ethyl acetate. The solution was washed with water and dried with magnesium sulfate. The drying agent was filtered off and the solvent was removed under reduced pressure. Purification by column chromatography (mobile phase: ethyl acetate/heptane 1:1) gave 6.95 g (84% of theory) of the title compound.

### Example A2

### 2-Amino4-(1-fluoro-1-methylethyl)-6-[N-(1-cyclobutyl-2-phenoxyethyl)-amino]-1,3,5-triazine (See Table 1, Example 178)

5.2 g (0.03 mol) of 2-amino4-chloro6-(1-fluoro-1-methylethyl)-1,3,5-triazine and 12.4 g (0.09 mol) of potassium carbonate were added to 5.1 g (0.026 mol) of (2-amino-2-cyclobutylethyl) phenyl ether in 40 ml of acetonitrile. The mxiture was stirred under reflux for 4 h. The reaction mixture was concentrated and the residue was taken up in ethyl acetate. The solution was washed with water and dried with magnesium sulfate. The drying agent was filtered off and the solvent was removed under reduced pressure. Purification by column chromatography (mobile phase: ethyl acetate/heptane 1:1) gave 7.95 g (88% of theory) of the title compound.

### Example A3

### 2-Amino4-(1-fluoro-1-methylethyl)-6-{N-[(1 R)-1-cyclobutyl-2-phenoxy-ethyl]amino}-1,3,5-triazine (See Table 2, Example 2.178)

5.2 g (0.03 mol) of 2-amino4-chloro-6-(1-fluoro-1-methylethyl)-1,3,5-triazine and 12.4 g (0.09 mol) of potassium carbonate were added to 5.1 g (0.026 mol) of (2R)-2-amino-2-cyclobutylethyl phenyl ether in 40 ml of acetonitrile. The reaction mixture was stirred under reflux for 4 h and the residue was then taken up in ethyl acetate and washed with water. The organic phase was dried with magnesium sulfate and filtered off, the solvent was removed under reduced pressure and the residue was chromatographed (mobile phase: ethyl acetate/heptane 1:1), giving 8.3 g (92% of theory) of the title compound.

### Example A4

2-Amino-4-(1-fluoro-1-methylethyl)6-{N-[(1S)- or (1 R)-1-cyclobutyl-2-phenoxyethyl]amino}-1,3,5-triazine (See Table 2, Example 2.178 and Table 3, Example 2.178, Respectibly),

2-Amino-4-(1-fluoro-1-methylethyl)6-{N-[(1RS)-1-cyclobutyl-2-phenoxy-ethyl]amino}-1,3,5-triazine (see Example A2) was chromatographed on a chiral stationary phase (Chiracel AD as carrier material, propan-2-ol/hexane 5/95 as mobile phase, 20° C., 0.5 ml/min) and detected by UV spectroscopy. The R and the S isomer were obtained in separated form. The absolute configuration was determined by comparison of the chromatography running times (RF) with the compound from Example A3.

The examplary compounds listed in Tables 1, 2 and 3 below are obtained analogously by the methods from Examples A1 and A2 or A3 and A4 and the methods referred to in the description.

| | |
|---|---|
| Ac | acetyl |
| Me | methyl |
| Et | ethyl |
| Pr | propyl |
| i-Pr | isopropyl |
| c-Pr | cyclopropyl |
| n-, i-, s-, t-bu | n-, iso, secundary or tertiary butyl |
| Ph | phenyl |
| m.p. melting point | |

For better legibility of the index numbers in the chemical formulae for the radicals in Table 1, the index numbers are of the same font size as the other text and not subscript. Thus, the formula CF₃ corresponds to the usual formula CF₃.in customary notation. In the table, the definition (X)ₙ=H corresponds to n=0

TABLE 1
Compounds of the formula (1) in racemic form

| | |
|---|---|
| 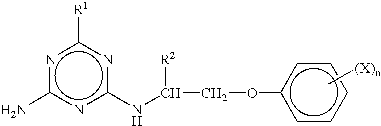 | (1) |

| | | | | Phys. |
|---|---|---|---|---|
| No. | (X)ₙ | R² | R¹ | data |
| 1 | 2,4-Cl | 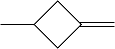 | CF3 | |
| 2 | 2,4-Cl | 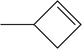 | i-propyl | |
| 3 | 2,4-Cl | 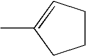 | CF(CH3)2 | |
| 4 | 2,4-Cl | 3,3-F2-c-butyl | i-propyl | |
| 5 | 2,4-F | 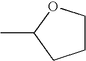 | CF2CHF2 | |
| 6 | 2,4-F | 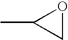 | i-propyl | |
| 7 | 2,4-F | CH2-c-butyl | CF(CH3)2 | |
| 8 | 2,4-F | 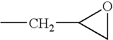 | CF(CH3)2 | |
| 9 | 2,5-F | 3,3-F2-c-butyl | CF(CH3)2 | |
| 10 | 2,5-F | 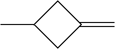 | i-propyl | |
| 11 | 2,5-F | 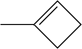 | CF(CH3)2 | |
| 12 | 2-Cl | 3,3-F2-c-butyl | CF2CHF2 | |
| 13 | 2-Cl | 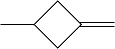 | i-propyl | |
| 14 | 2-Cl | 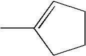 | i-propyl | |
| | 2-Cl | 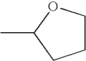 | i-propyl | |
| 15 | | | | |
| 16 | 2-Cl,4-NO2 | 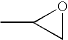 | i-propyl | |
| 17 | 2-Cl,4-NO2 | 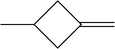 | i-propyl | |
| 18 | 2-F | 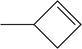 | CF(CH3)2 | |
| 19 | 2-F | 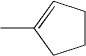 | i-propyl | |
| 20 | 2-F | 3,3-F2-c-butyl | i-propyl | |
| 21 | 2-F | 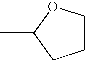 | i-propyl | |
| 22 | 2-Me | 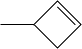 | CF(CH3)2 | |
| 23 | 2-Me | 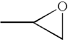 | i-propyl | |
| 24 | 2-Me | CH2-c-butyl | i-propyl | |
| 25 | 2-Me | 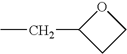 | i-propyl | |
| 26 | 3,4-F | 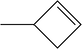 | CHFCF3 | |
| 27 | 3,4-F | 2,2,3,3-F4-c-butyI | i-propyl | |
| 28 | 3,4-F | 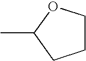 | i-propyl | |
| 29 | 3,4-F | c-butyl | CF(CH3)2 | m.p. 56 |
| | | | | ° C. |
| 30 | 3,4-F | c-butyl | CF2CHF2 | resin |
| 31 | 3,4-F | c-butyl | CHFCH3 | resin |
| 32 | 3,4-F | c-butyl | i-propyl | resin |
| 33 | 3,4-F | 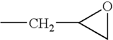 | CF(CH3)2 | |
| 34 | 3,5-CF3 | 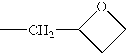 | i-propyl | |
| 35 | 3,5-Cl | 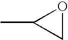 | CHFCH3 | |
| 36 | 3,5-Cl | 2,2,3,3-F4-c-butyl | i-propyl | |
| 37 | 3,5-Cl | 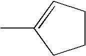 | i-propyl | |
| 38 | 3,5-Cl | 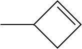 | i-propyl | |
| 39 | 3,5-F | c-butyl | i-propyl | resin |
| 40 | 3,5-F | c-butyl | CF(CH3)2 | resin |
| 41 | 3,5-F | 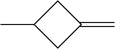 | CF2CHF2 | |
| 42 | 3,5-F | 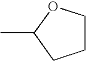 | CF2CHF2 | |
| 43 | 3,5-F | CH2-c-propyl | CF(CH3)2 | |
| 44 | 3,5-F | CH2-c-butyl | CF(CH3)2 | |
| 45 | 3,5-Me | c-butyl | CF(CH3)2 | resin |
| 46 | 3,5-Me | 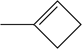 | CF(CH3)2 | |
| 47 | 3,5-Me | 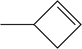 | CF2CHF2 | |
| 48 | 3,5-Me | 2,2,3,3-F4-c-butyl | CF2CHF2 | |
| 49 | 3,5-Me | 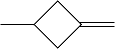 | CF2CHF2 | |
| 50 | 3,5-Me | c-butyl | CHFCH3 | resin |
| 51 | 3,5-Me | c-butyl | i-propyl | resin |
| 52 | 3,5-OMe | 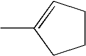 | CF(CH3)2 | |
| 53 | 3,5-OMe | 3,3-F2-c-butyl | CF3 | |
| 54 | 3,5-OMe | 2,2,3,3-F4-c-butyl | CHFCH3 | |
| 55 | 3,5-OMe | 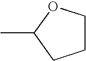 | CHFCH3 | |
| 56 | 3,5-OMe | 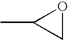 | CHFCH3 | |
| 57 | 3,5-OMe | 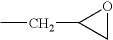 | CF(CH3)2 | |
| 58 | 3-CF3 | 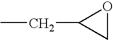 | CF3 | |
| 59 | 3-CF3 | 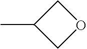 | CHFCH3 | |
| 60 | 3-CF3 | 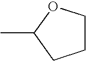 | i-propyl | |
| 61 | 3-CF3 | 2,2,3,3-F4-c-butyl | i-propyl | |
| 62 | 3-CF3 | 3,3-F2-c-butyl | i-propyl | |
| 63 | 3-CF3 | 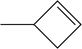 | i-propyl | |
| 64 | 3-Cl | c-butyl | CF(CH3)2 | resin |
| 65 | 3-Cl | 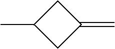 | CF(CH3)2 | |
| 66 | 3-Cl | 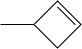 | CF(CH3)2 | |
| 67 | 3-Cl | 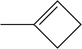 | CF(CH3)2 | |
| 68 | 3-Cl | c-butyl | CF2CHF2 | resin |
| 69 | 3-Cl | CH2-c-butyl | CF(CH3)2 | |
| 70 | 3-Cl | CH2-c-propyl | OF(CH3)2 | |
| 71 | 3-Cl | c-butyl | CHFCH3 | resin |
| 72 | 3-Cl | 3,3-F2-c-butyl | CF3 | |
| 73 | 3-Cl | c-butyl | i-propyl | resin |
| 74 | 3-Cl | 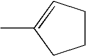 | i-propyl | |
| 75 | 3-Cl | 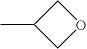 | i-propyl | |
| 76 | 3-Cl | 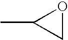 | i-propyl | |
| 77 | 3-Cl,5-Me | c-butyl | CF2CHF2 | |
| 78 | 3-CI,5-Me | 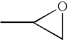 | CF3 | |
| 79 | 3-CI,5-Me | 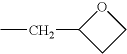 | CHFCH3 | |
| 80 | 3-F | 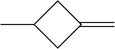 | i-propyl | |
| 81 | 3-F | 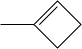 | i-propyl | |
| 82 | 3-F | 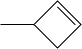 | i-propyl | |
| 83 | 3-F | c-butyl | CF(CH3)2 | resin |
| 84 | 3-F | 3,3-F2-c-butyl | OF3 | |
| 85 | 3-F | c-butyl | CHFCH3 | resin |
| 86 | 3-F | c-butyl | i-propyl | resin |
| 87 | 3-F | c-butyl | CCI2CH3 | resin |
| 88 | 3-F | c-butyl | CCI(CH3)2 | resin |
| 89 | 3-F | CH2-c-propyl | CF(CH3)2 | |
| 90 | 3-F | CH2-c-butyl | CF(CH3)2 | |
| 91 | 3-F5-Me | 3,3-F2-c-butyl | CF(CH3)2 | |
| 92 | 3-F,5-Me | 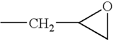 | CF(CH3)2 | |
| 93 | 3-F,5-Me | 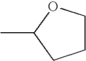 | CHFCH3 | |
| 94 | 3-F,5-Me | CH2-c-pentyl | i-propyl | |
| 95 | 3-F,5-Me | 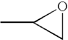 | i-propyl | |
| 96 | 3-F,5-OMe | 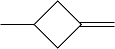 | CF(CH3)2 | |
| 97 | 3-F,5-OMe | 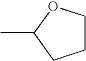 | CF2CHF2 | |
| 98 | 3-F,5-OMe | 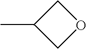 | CF2OHF2 | |
| 99 | 3-F,5-OMe | 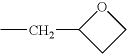 | CF(CH3)2 | |
| 100 | 3-Me | c-butyl | CF(CH3)2 | resin |
| 101 | 3-Me | 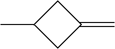 | CF(CH3)2 | |
| 102 | 3-Me | c-butyl | CF2CHF2 | resin |
| 103 | 3-Me | 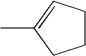 | CF2CHF2 | |
| 104 | 3-Me | c-butyl | CHFCH3 | resin |
| 105 | 3-Me | CH2-c-butyl | CF(CH3)2 | |
| 106 | 3-Me | 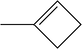 | CF3 | |
| 107 | 3-Me | 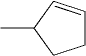 | CF3 | |
| 108 | 3-Me | c-butyt | i-propyt | resin |
| 109 | 3-Me | c-pentyl | CF(CH3)2 | m.p. |
| | | | | 138° C. |
| 110 | 3-Me | c-pentyl | CF2CHF2 | m.p. |
| | | | | 154° C. |
| 111 | 3-Me | 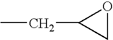 | CF(CH3)2 | |
| 112 | 3-Me | c-pentyl | i-propyl | resin |
| 113 | 3-Me | c-hexyl | i-propyl | resin |
| 114 | 3-Me | c-hexyl | CF(CH3)2 | m.p. |
| | | | | 147° C. |
| 115 | 3-Me,4-F | c-butyl | CF(CH3)2 | resin |
| 116 | 3-Me,4-F | 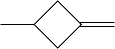 | CF(CH3)2 | |
| 117 | 3-Me,4-F | 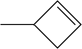 | CF(CH3)2 | |
| 118 | 3-Me,4-F | c-butyl | CHFOH3 | resin |
| 119 | 3-Me,4-F | 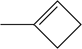 | CHFCH3 | |
| 120 | 3-Me,4-F | c-butyl | CF2CHF2 | resin |
| 121 | 3-Me,4-F | c-butyl | i-propyl | resin |
| 122 | 3-Me,4-F | CH2-c-butyl | OF(CH3)2 | |
| 123 | 3-Me,4-F | CH2-c-propyl | OF(CH3)2 | |
| 124 | 3-Me,4-F | 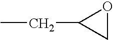 | CF(CH3)2 | |
| 125 | 3-Me,4-F | 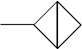 | i-propyl | |
| 126 | 3-OEt | 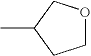 | CF(CH3)2 | |
| 127 | 3-OEt | 3,3-(CH3)2-c-butyl | CF(CH3)2 | |
| 128 | 3-OEt | 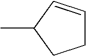 | CF3 | |
| 129 | 3-OEt | 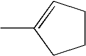 | i-propyl | |
| 130 | 3-OMe | c-butyt | CF(CH3)2 | resin |
| 131 | 3-OMe | 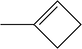 | CF3 | |
| 132 | 3-OMe | c-butyl | CHFCH3 | resin |
| 133 | 3-OMe | c-butyl | i-propyl | resin |
| 134 | 3-OMe | c-butyl | CCl2CH3 | resin |
| 135 | 3-OMe | c-butyl | CCl(CH3)2 | resin |
| 136 | 3-OMe,5-Cl | 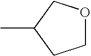 | CHFCH3 | |
| 137 | 3-OMe,5-Cl | 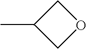 | CHFCH3 | |
| 138 | 3-OMe,5-Cl | CH2-c-pentyl | CHFCH3 | |
| 139 | 4-Br | 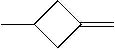 | CF(CH3)2 | |
| 140 | 4-Br | 3,3-(C H3)2-c-butyl | CF2CHF2 | |
| 141 | 4-Br | 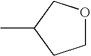 | CHFCH3 | |
| 142 | 4-Br | 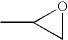 | i-propyl | |
| 143 | 4-Cl | 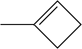 | CF(CH3)2 | |
| 144 | 4-Cl | 3,3-(CH3)2-c-butyl | CF2CHF2 | |
| 145 | 4-Cl | 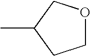 | CHFCH3 | |
| 146 | 4-Cl | 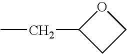 | i-propyl | |
| 147 | 4-Cl | 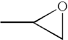 | i-propyl | |
| 148 | 4-Cl | 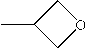 | i-propyl | |
| 149 | 4-Cl | 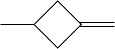 | i-propyl | |
| 150 | 4-F | 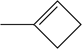 | i-propyl | |
| 151 | 4-F | 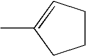 | i-propyl | |
| 152 | 4-F | c-butyl | CF(CH3)2 | m.p. 55 |
| | | | | ° C. |
| 153 | 4-F | c-butyl | CF2CHF2 | |
| 154 | 4-F | c-butyl | CHFCH3 | resin |
| 155 | 4-F | c-butyl | i-propyl | resin |
| 156 | 4-F | 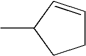 | i-propyl | |
| 157 | 4-F | 3,3-(CH3)2-c-butyl | i-propyl | |
| 158 | 3-Cl, 5-F | 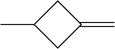 | i-propyl | |
| 159 | 3-Cl, 5-F | 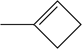 | i-propyl | |
| 160 | 3-Cl, 5-F | 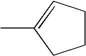 | i-propyl | |
| 161 | 3-Cl, 5-F | c-butyl | CF(CH3)2 | |
| 162 | 3-Cl, 5-F | c-butyl | CF2CHF2 | |
| 163 | 3-Cl, 5-F | c-butyl | CHFCH3 | |
| 164 | 3-Cl, 5-F | c-butyl | i-propyl | |
| 165 | 3-Cl, 5-F | 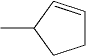 | i-propyl | |
| 166 | 3-Cl, 5-F | 3,3-(CH3)2-c-butyl | i-propyl | |
| 167 | 4-F,5-CF3 | 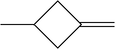 | i-propyl | |
| 168 | 4-Me | CH2-c-pentyl | CF(CH3)2 | |
| 169 | 4-Me | 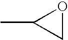 | i-propyl | |
| 170 | 4-Me | 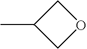 | i-propyl | |
| 171 | 4-Me | 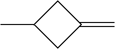 | i-propyl | |
| 172 | 4-Me | 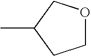 | i-propyl | |
| 173 | 4-Me | 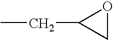 | CF(CH3)2 | |
| 174 | 4-OCF3 | 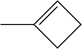 | CF(CH3)2 | |
| 175 | 4-OCF3 | 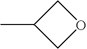 | i-propyl | |
| 176 | 4-OCF3 | 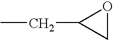 | CF(CH3)2 | |
| 177 | 4-OCF3 | 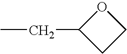 | i-propyl | |
| 178 | H | c-butyl | CF(OH3)2 | resin |
| 179 | H | 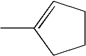 | CF(CH3)2 | |
| 180 | H | 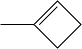 | CF(CH3)2 | |
| 181 | H | 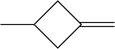 | CF(CH3)2 | |
| 182 | H | c-butyl | CF2CHF2 | resin |
| 183 | H | c-butyl | CHFCH3 | resin |
| 184 | H | 3,3-(CH3)2-c-butyI | CHFCH3 | |
| 185 | H | c-butyl | i-propyl | resin |
| 186 | H | 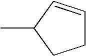 | i-propyl | |
| 187 | H | 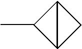 | i-propyl | |
| 188 | H | 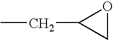 | CF(CH3)2 | |
| 189 | H | CH2-c-propyl | CF(CH3)2 | |
| 190 | H | CH2-c-butyl | CF(CH3)2 | |
| 191 | H | CH2-c-butyl | CF2CHF2 | |
| 192 | 2-F | 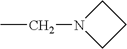 | CF(CH3)2 | |
| 193 | 2-F | 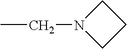 | i-propyl | |
| 194 | 2-F | 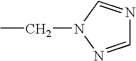 | i-propyl | |
| 195 | 2-F | 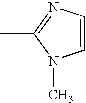 | i-propyl | |
| 196 | 2-Me | 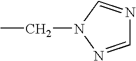 | CF(CH3)2 | |
| 197 | 2-Me | 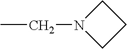 | i-propyl | |
| 198 | 2-Me | 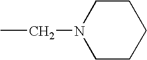 | i-propyl | |
| 199 | 2-Me | 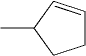 | i-propyl | |
| 200 | 3,4-F | 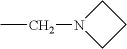 | CHFCF3 | |
| 201 | 3,4-F | 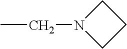 | i-propyl | |
| 202 | 3,4-F | 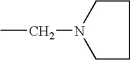 | i-propyl | |
| 203 | 3,4-F | 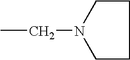 | CF(CH3)2 | |
| 204 | 3,4-F | 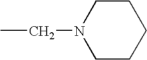 | CF2CHF2 | |
| 205 | 3,4-F | 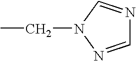 | CHFOH3 | |
| 206 | 3,4-F | 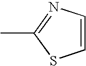 | i-propyl | |
| 207 | 3,4-F | 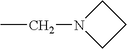 | CF(CH3)2 | |
| 208 | 3,5-F | 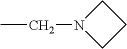 | i-propyl | |
| 209 | 3,5-F | 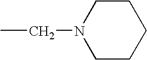 | CF(CH3)2 | |
| 210 | 3,5-F | 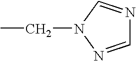 | CF2CHF2 | |
| 211 | 3,5-F | 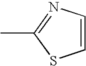 | CF2CHF2 | |
| 212 | 3,5-F | 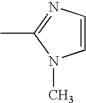 | CF(CH3)2 | |
| 213 | 3,5-F | 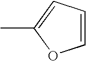 | CF(CH3)2 | |
| 214 | 3,5-Me | 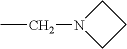 | CF(CH3)2 | |
| 215 | 3,5-Me | 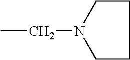 | CF(CH3)2 | |
| 216 | 3,5-Me | 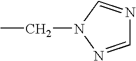 | CF2CHF2 | |
| 217 | 3,5-Me | 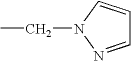 | CF2CHF2 | |
| 218 | 3,5-Me | 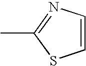 | CF2CHF2 | |
| 219 | 3,5-Me | 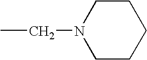 | CHFCH3 | |
| 220 | 3,5-Me | 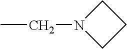 | i-propyl | |
| 221 | 3,5-OMe | 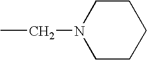 | CF(CH3)2 | |
| 222 | 3,5-OMe | 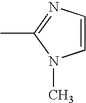 | CF3 | |
| 223 | 3,5-OMe | 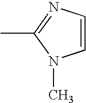 | CHFCH3 | |
| 224 | 3,5-OMe | 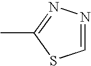 | CHFCH3 | |
| 225 | 3,5-OMe | 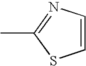 | CHFCH3 | |
| 226 | 3-CF3 | 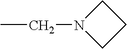 | CF3 | |
| 227 | 3-CF3 | 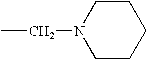 | CHFCH3 | |
| 228 | 3-CF3 | 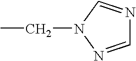 | i-propyl | |
| 229 | 3-CF3 | 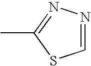 | i-propyl | |
| 230 | 3-CF3 | 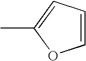 | i-propyl | |
| 231 | 3-Cl | 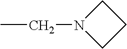 | CF(CH3)2 | |
| 232 | 3-Cl | 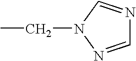 | CF(CH3)2 | |
| 233 | 3-Cl | 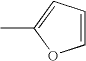 | CF(CH3)2 | |
| 234 | 3-Cl | 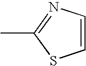 | CF2CHF2 | |
| 235 | 3-Cl | 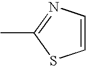 | CF(CH3)2 | |
| 236 | 3-Cl | 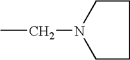 | CF(CH3)2 | |
| 237 | 3-Cl | 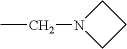 | CHFCH3 | |
| 238 | 3-Cl | 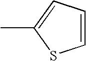 | CF3 | |
| 239 | 3-Cl | 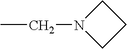 | i-propyl | |
| 240 | 3-Cl | 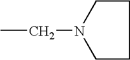 | i-propyl | |
| 241 | 3-Cl | 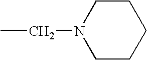 | i-propyl | |
| 242 | 3-Cl | 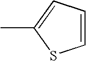 | i-propyl | |
| 243 | 3-F | 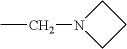 | i-propyl | |
| 244 | 3-F | 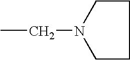 | i-propyl | |
| 245 | 3-F | 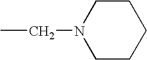 | i-propyl | |
| 246 | 3-F | 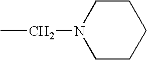 | CF(CH3)2 | |
| 247 | 3-F | 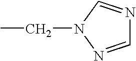 | CF3 | |
| 248 | 3-F | 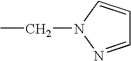 | CHFCH3 | |
| 249 | 3-F | 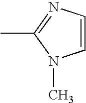 | i-propyl | oil |
| 250 | 3-F | 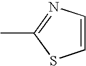 | CCl2CH3 | |
| 251 | 3-F | 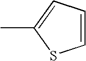 | CCl(CH3)2 | |
| 252 | 3-F | 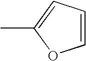 | CF(CH3)2 | |
| 253 | 3-F | 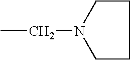 | CF(CH3)2 | |
| 254 | 3-F,5-Me | 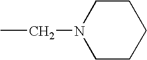 | CF(CH3)2 | |
| 255 | 3-F,5-Me | 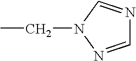 | CF(CH3)2 | |
| 256 | 3-F,5-Me | 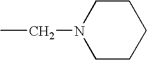 | i-propyl | |
| 257 | 3-F,5-OMe | 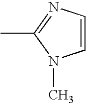 | CF(OH3)2 | |
| 258 | 3-F,5-OMe | 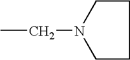 | CF2CHF2 | |
| 259 | 3-F,5-OMe | 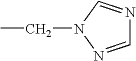 | CF(CH3)2 | |
| 260 | 3-Me | 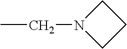 | CF(CH3)2 | |
| 261 | 3-Me | 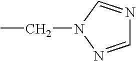 | CF(CH3)2 | |
| 262 | 3-Me | 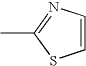 | CF2CHF2 | |
| 263 | 3-Me | 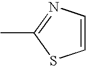 | CF2CHF2 | |
| 264 | 3-Me | 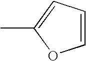 | CHFCH3 | |
| 265 | 3-Me | 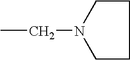 | CF3 | |
| 266 | 3-Me | 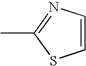 | CF3 | |
| 267 | 3-Me | 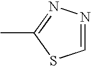 | i-propyl | |
| 268 | 3-Me | 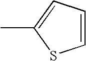 | CF(CH3)2 | |
| 269 | 3-Me | 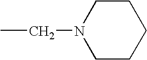 | CF(CH3)2 | |
| 270 | 3-Me | 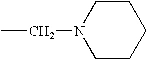 | i-propyl | |
| 271 | 3-Me | 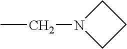 | i-propyl | |
| 272 | 3-Me | 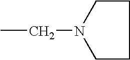 | CF(CH3)2 | |
| 273 | 3-Me,4-F | 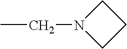 | CF(CH3)2 | |
| 274 | 3-Me~4-F | 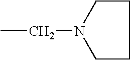 | CF(CH3)2 | |
| 275 | 3-Me,4-F | 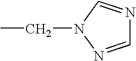 | CF(CH3)2 | |
| 276 | 3-Me,4-F | 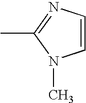 | CHFCH3 | |
| 277 | 3-Me,4-F | 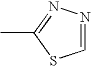 | CHFCH3 | |
| 278 | 3-Me,4-F | 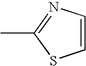 | CF2OHF2 | |
| 279 | 3-Me,4-F | 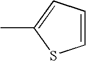 | i-propyl | |
| 280 | 3-Me,4-F | 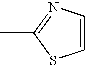 | CF(CH3)2 | |
| 281 | 3-Me,4-F | 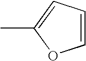 | CF(CH3)2 | |
| 282 | 3-Me,4-F | 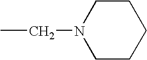 | CF(CH3)2 | |
| 283 | 3-Me,4-F | 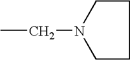 | i-propyl | |
| 284 | 4-Cl | 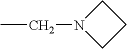 | i-propyl | |
| 285 | 4-Cl | 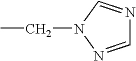 | i-propyl | |
| 286 | 4-F | 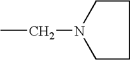 | i-propyl | |
| 287 | 4-F | 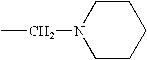 | i-propyl | |
| 288 | 4-F | 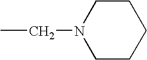 | CF(CH3)2 | |
| 289 | 4-F | 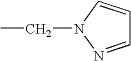 | CF2CHF2 | |
| 290 | 4-F | 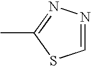 | i-propyl | |
| 291 | 4-F | 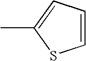 | i-propyl | |
| 292 | 4-F | 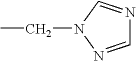 | i-propyl | |
| 293 | 3-Cl, 5-F | 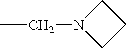 | i-propyl | |
| 294 | 3-Cl, 5-F | 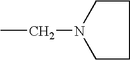 | i-propyl | |
| 295 | 3-Cl, 5-F | 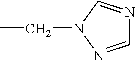 | i-propyl | |
| 296 | 3-Cl, 5-F | 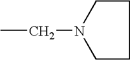 | CF(CH3)2 | |
| 297 | 3-Cl, 5-F | 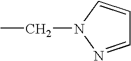 | CF2CHF2 | |
| 298 | 3-Cl, 5-F | 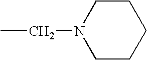 | CHFCH3 | |
| 299 | 3-Cl, 5-F | 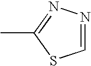 | i-propyl | |
| 300 | 3-Cl, 5-F | 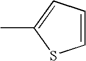 | i-propyl | |
| 301 | 3-Cl, 5-F | 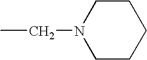 | i-propyl | |
| 302 | 4-F,5-CF3 | 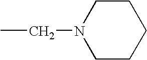 | i-propyl | |
| 303 | H | 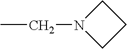 | CF(CH3)2 | |
| 304 | H | 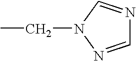 | CF(CH3)2 | oil |
| 305 | H | 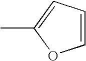 | CF(CH3)2 | |
| 306 | H | 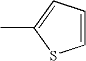 | CF(OH3)2 | |
| 307 | H | 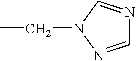 | CF2CHF2 | Oil |
| 308 | H | 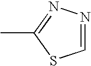 | CHFCH3 | |
| 309 | H | 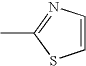 | CHFCH3 | |
| 310 | H | 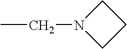 | i-propyl | |
| 311 | H | 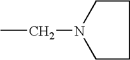 | i-propyl | |
| 312 | H | 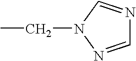 | i-propyl | oil |
| 313 | H | 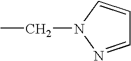 | CF(CH3)2 | |
| 314 | H | 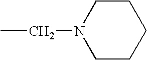 | CF(CH3)2 | |
| 315 | H | 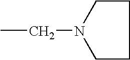 | CF(CH3)2 | |
| 316 | H | 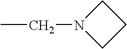 | CF2CHF2 | |

TABLE 2
Compounds of the formula (2)
(in each case the R isomer at the carbon atom which is
substituted by R² if the priority of the radicals is as follows:
1. NH, 2. R², 3. CH₂OPh(X)ₙ, 4. H)

| | |
|---|---|
| 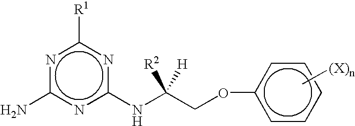 | (2) |

| | | | | Phys. |
|---|---|---|---|---|
| No. | (X)ₙ | R² | R¹ | data |
| 2.1 | 2,4-Cl | 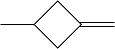 | CF3 | |
| 2.2 | 2,4-Cl | 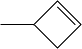 | i-propyl | |
| 2.3 | 2,4-Cl | 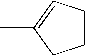 | CF(CH3)2 | |
| 2.4 | 2,4-Cl | 3,3-F2-c-butyl | i-propyl | |
| 2.5 | 2,4-F | 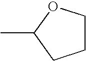 | CF2CHF2 | |
| 2.6 | 2,4-F | 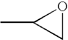 | i-propyl | |
| 2.7 | 2,4-F | CH2-c-butyl | CF(CH3)2 | |
| 2.8 | 2,4-F | 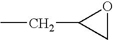 | CF(CH3)2 | |
| 2.9 | 2,5-F | 3,3-F2-c-butyl | CF(CH3)2 | |
| 2.10 | 2,5-F | 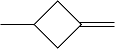 | i-propyl | |
| 2.11 | 2,5-F | 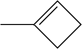 | CF(CH3)2 | |
| 2.12 | 2-Cl | 3,3-F2-c-butyl | CF2CHF2 | |
| 2.13 | 2-Cl | 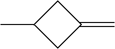 | i-propyl | |
| 2.14 | 2-Cl | 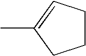 | i-propyl | |
| 2.15 | 2-Cl | 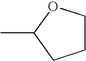 | i-propyl | |
| 2.16 | 2-Cl,4-NO2 | 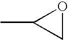 | i-propyl | |
| 2.17 | 2-Cl,4-NO2 | 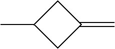 | i-propyl | |
| 2.18 | 2-F | 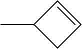 | CF(CH3)2 | |
| 2.19 | 2-F | 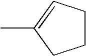 | i-propyl | |
| 2.20 | 2-F | 3,3-F2-c-butyl | i-propyl | |
| 2.21 | 2-F | 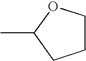 | i-propyl | |
| 2.22 | 2-Me | 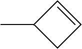 | CF(CH3)2 | |
| 2.23 | 2-Me | 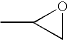 | i-propyl | |
| 2.24 | 2-Me | CH2-c-butyl | i-propyl | |
| 2.25 | 2-Me | 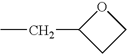 | i-propyl | |
| 2.26 | 3,4-F | 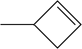 | CHFCF3 | |
| 2.27 | 3,4-F | 2,2,3,3-F4-c-butyl | i-propyl | |
| 2.28 | 3,4-F | 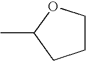 | i-propyl | |
| 2.29 | 3,4-F | c-butyl | CF(CH3)2 | crystalline |
| 2.30 | 3,4-F | c-butyl | CF2CHF2 | resin |
| 2.31 | 3,4-F | c-butyl | CHFCH3 | resin |
| 2.32 | 3,4-F | c-butyl | i-propyl | resin |
| 2.33 | 3,4-F | 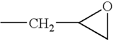 | CF(CH3)2 | |
| 2.34 | 3,5-CF3 | 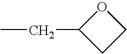 | i-propyl | |
| 2.35 | 3,5-Cl | 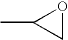 | CHFCH3 | |
| 2.36 | 3,5-Cl | 2,2,3,3-F4-c-butyl | i-propyl | |
| 2.37 | 3,5-Cl | 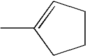 | i-propyl | |
| 2.38 | 3,5-Cl | 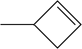 | i-propyl | |
| 2.39 | 3,5-F | c-butyl | i-propyl | resin |
| 2.40 | 3,5-F | c-butyl | CF(CH3)2 | resin |
| 2.41 | 3,5-F | 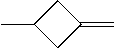 | CF2CHF2 | |
| 2.42 | 3,5-F | 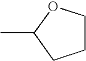 | CF2CHF2 | |
| 2.43 | 3,5-F | CH2-c-propyl | CF(CH3)2 | |
| 2.44 | 3,5-F | CH2-c-butyl | CF(CH3)2 | |
| 2.45 | 3,5-Me | c-butyl | CF(CH3)2 | resin |
| 2.46 | 3,5-Me | 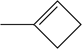 | CF(CH3)2 | |
| 2.47 | 3,5-Me | 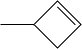 | CF2CHF2 | |
| 2.48 | 3,5-Me | 2,2,3,3-F4-c-butyl | CF2CHF2 | |
| 2.49 | 3,5-Me | 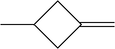 | CF2CHF2 | |
| 2.50 | 3,5-Me | c-butyl | CHFCH3 | resin |
| 2.51 | 3,5-Me | c-butyl | i-propyl | resin |
| 2.52 | 3,5-OMe | 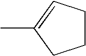 | CF(CH3)2 | |
| 2.53 | 3,5-OMe | 3,3-F2-c-butyl | CF3 | |
| 2.54 | 3,5-OMe | 2,2,3,3-F4-c-butyl | CHFCH3 | |
| 2.55 | 3,5-OMe | 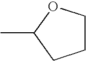 | CHFCH3 | |
| 2.56 | 3,5-OMe | 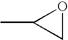 | CHFCH3 | |
| 2.57 | 3,5-OMe | 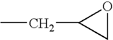 | CF(CH3)2 | |
| 2.58 | 3-CF3 | 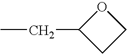 | CF3 | |
| 2.59 | 3-CF3 | 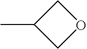 | CHFCH3 | |
| 2.60 | 3-CF3 | 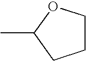 | i-propyl | |
| 2.61 | 3-CF3 | 2,2,3,3-F4-c-butyl | i-propyl | |
| 2.62 | 3-CF3 | 3,3-F2-c-butyl | i-propyl | |
| 2.63 | 3-CF3 | 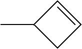 | i-propyl | |
| 2.64 | 3-Cl | c-butyl | CF(CH3)2 | resin |
| 2.65 | 3-Cl | 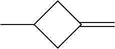 | CF(CH3)2 | |
| 2.66 | 3-Cl | 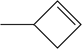 | CF(CH3)2 | |
| 2.67 | 3-Cl | 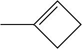 | CF(CH3)2 | |
| 2.68 | 3-Cl | c-butyl | CF2CHF2 | resin |
| 2.69 | 3-Cl | CH2-c-butyl | CF(CH3)2 | |
| 2.70 | 3-Cl | CH2-c-propyl | CF(CH3)2 | |
| 2.71 | 3-Cl | c-butyl | CHFCH3 | resin |
| 2.72 | 3-Cl | 3,3-F2-c-butyl | CF3 | |
| 2.73 | 3-Cl | c-butyl | i-propyl | resin |
| 2.74 | 3-Cl | 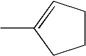 | i-propyl | |
| 2.75 | 3-Cl | 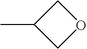 | i-propyl | |
| 2.76 | 3-Cl | 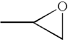 | i-propyl | |
| 2.77 | 3-Cl,5-Me | c-butyl | CF2CHF2 | |
| 2.78 | 3-Cl,5-Me | 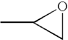 | CF3 | |
| 2.79 | 3-Cl,5-Me | 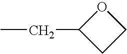 | CHFCH3 | |
| 2.80 | 3-F | 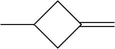 | i-propyl | |
| 2.81 | 3-F | 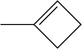 | i-propyl | |
| 2.82 | 3-F | 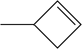 | i-propyl | |
| 2.83 | 3-F | c-butyl | CF(CH3)2 | resin |
| 2.84 | 3-F | 3,3-F2-c-butyl | CF3 | |
| 2.85 | 3-F | c-butyl | CHFCH3 | resin |
| 2.86 | 3-F | c-butyl | i-propyl | resin |
| 2.87 | 3-F | c-butyl | CCl2CH3 | resin |
| 2.88 | 3-F | c-butyl | CCl(CH3)2 | resin |
| 2.89 | 3-F | CH2-c-propyl | CF(CH3)2 | |
| 2.90 | 3-F | CH2-c-butyl | CF(CH3)2 | |
| 2.91 | 3-F5-Me | 3,3-F2-c-butyl | CF(CH3)2 | |
| 2.92 | 3-F,5-Me | 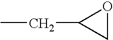 | CF(CH3)2 | |
| 2.93 | 3-F,5-Me | 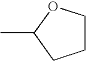 | CHFCH3 | |
| 2.94 | 3-F,5-Me | CH2-c-pentyl | i-propyl | |
| 2.95 | 3-F,5-Me | 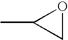 | i-propyl | |
| 2.96 | 3-F,5-OMe | 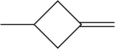 | CF(CH3)2 | |
| 2.97 | 3-F,5-OMe | 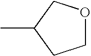 | CF2CHF2 | |
| 2.98 | 3-F,5-OMe | 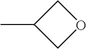 | CF2CHF2 | |
| 2.99 | 3-F,5-OMe | 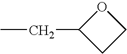 | CF(CH3)2 | |
| 2.100 | 3-Me | c-butyl | CF(CH3)2 | resin |
| 2.101 | 3-Me | 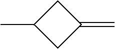 | CF(CH3)2 | |
| 2.102 | 3-Me | c-butyl | CF2CHF2 | resin |
| 2.103 | 3-Me | 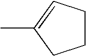 | CF2CHF2 | |
| 2.104 | 3-Me | c-butyl | CHFCH3 | resin |
| 2.105 | 3-Me | CH2-c-butyl | CF(CH3)2 | |
| 2.106 | 3-Me | 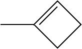 | CF3 | |
| 2.107 | 3-Me | 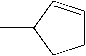 | CF3 | |
| 2.108 | 3-Me | c-butyl | i-propyl | resin |
| 2.109 | 3-Me | c-pentyl | CF(CH3)2 | crystalline |
| 2.110 | 3-Me | c-pentyl | CF2CHF2 | crystalline |
| 2.111 | 3-Me | 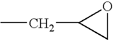 | CF(CH3)2 | |
| 2.112 | 3-Me | c-pentyl | i-propyl | resin |
| 2.113 | 3-Me | c-hexyl | i-propyl | resin |
| 2.114 | 3-Me | c-hexyl | CF(CH3)2 | crystalline |
| 2.115 | 3-Me,4-F | c-butyl | CF(CH3)2 | resin |
| 2.116 | 3-Me,4-F | 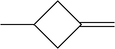 | CF(CH3)2 | |
| 2.117 | 3-Me,4-F | 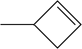 | CF(CH3)2 | |
| 2.118 | 3-Me,4-F | c-butyl | CHFOH3 | resin |
| 2.119 | 3-Me,4-F | 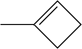 | CHFCH3 | |
| 2.120 | 3-Me,4-F | c-butyl | CF2CHF2 | resin |
| 2.121 | 3-Me,4-F | c-butyl | i-propyl | resin |
| 2.122 | 3-Me,4-F | CH2-c-butyl | CF(CH3)2 | |
| 2.123 | 3-Me,4-F | CH2-c-propyl | CF(CH3)2 | |
| 2.124 | 3-Me,4-F | 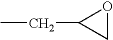 | CF(CH3)2 | |
| 2.125 | 3-Me,4-F | 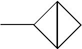 | i-propyl | |
| 2.126 | 3-OEt | 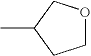 | CF(CH3)2 | |
| 2.127 | 3-OEt | 3,3-(CH3)2-c-butyl | CF(CH3)2 | |
| 2.128 | 3-OEt | 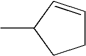 | CF3 | |
| 2.129 | 3-OEt | 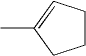 | i-propyl | |
| 2.130 | 3-OMe | c-butyt | CF(CH3)2 | resin |
| 2.131 | 3-OMe | 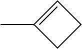 | CF3 | |
| 2.132 | 3-OMe | c-butyl | CHFCH3 | resin |
| 2.133 | 3-OMe | c-butyl | i-propyl | resin |
| 2.134 | 3-OMe | c-butyl | CCl2CH3 | resin |
| 2.135 | 3-OMe | c-butyl | CCl(CH3)2 | resin |
| 2.136 | 3-OMe,5-Cl | 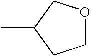 | CHFCH3 | |
| 2.137 | 3-OMe,5-OI | 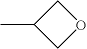 | CHFCH3 | |
| 2.138 | 3-OMe,5-Cl | CH2-c-pentyl | CHFCH3 | |
| 2.139 | 4-Br | 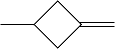 | CF(CH3)2 | |
| 2.140 | 4-Br | 3,3-(C H3)2-c-butyl | CF2CHF2 | |
| 2.141 | 4-Br | 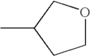 | CHFCH3 | |
| 2.142 | 4-Br | 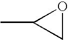 | i-propyl | |
| 2.143 | 4-Cl | 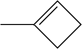 | CF(CH3)2 | |
| 2.144 | 4-Cl | 3,3-(CH3)2-c-butyl | CF2CHF2 | |
| 2.145 | 4-Cl | 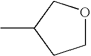 | CHFCH3 | |
| 2.146 | 4-Cl | 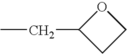 | i-propyl | |
| 2.147 | 4-Cl | 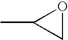 | i-propyl | |
| 2.148 | 4-Cl | 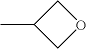 | i-propyl | |
| 2.149 | 4-Cl | 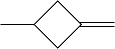 | i-propyl | |
| 2.150 | 4-F | 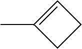 | i-propyl | |
| 2.151 | 4-F | 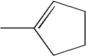 | i-propyl | |
| 2.152 | 4-F | c-butyl | CF(CH3)2 | crystalline |
| 2.153 | 4-F | c-butyl | CF2CHF2 | |
| 2.154 | 4-F | c-butyl | CHFCH3 | resin |
| 2.155 | 4-F | c-butyl | i-propyl | resin |
| 2.156 | 4-F | 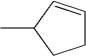 | i-propyl | |
| 2.157 | 4-F | 3,3-(CH3)2-c-butyl | i-propyl | |
| 2.158 | 3-Cl, 5-F | 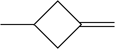 | i-propyl | |
| 2.159 | 3-Cl, 5-F | 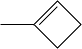 | i-propyl | |
| 2.160 | 3-Cl, 5-F | 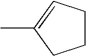 | i-propyl | |
| 2.161 | 3-Cl, 5-F | c-butyl | CF(CH3)2 | |
| 2.162 | 3-Cl, 5-F | c-butyl | CF2CHF2 | |
| 2.163 | 3-Cl, 5-F | c-butyl | CHFCH3 | |
| 2.164 | 3-Cl, 5-F | c-butyl | i-propyl | |
| 2.165 | 3-Cl, 5-F | 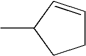 | i-propyl | |
| 2.166 | 3-Cl, 5-F | 3,3-(CH3)2-c-butyl | i-propyl | |
| 2.167 | 4-F,5-CF3 | 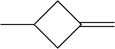 | i-propyl | |
| 2.168 | 4-Me | CH2-c-pentyl | CF(CH3)2 | |
| 2.169 | 4-Me | 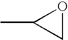 | i-propyl | |
| 2.170 | 4-Me | 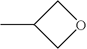 | i-propyl | |
| 2.171 | 4-Me | 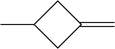 | i-propyl | |
| 2.172 | 4-Me | 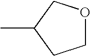 | i-propyl | |
| 2.173 | 4-Me | 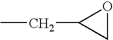 | CF(CH3)2 | |
| 2.174 | 4-OCF3 | 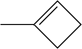 | CF(CH3)2 | |
| 2.175 | 4-OCF3 | 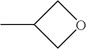 | i-propyl | |
| 2.176 | 4-OCF3 | 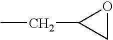 | CF(CH3)2 | |
| 2.177 | 4-OCF3 | 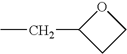 | i-propyl | |
| 2.178 | H | c-butyl | CF(OH3)2 | resin |
| 2.179 | H | 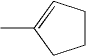 | CF(CH3)2 | |
| 2.180 | H | 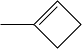 | CF(CH3)2 | |
| 2.181 | H | 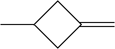 | CF(CH3)2 | |
| 2.182 | H | c-butyl | CF2CHF2 | resin |
| 2.183 | H | c-butyl | CHFCH3 | resin |
| 2.184 | H | 3,3-(CH3)2-c-butyl | CHFCH3 | |
| 2.185 | H | c-butyl | i-propyl | resin |
| 2.186 | H | 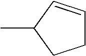 | i-propyl | |
| 2.187 | H | 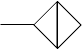 | i-propyl | |
| 2.188 | H | 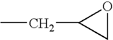 | CF(CH3)2 | |
| 2.189 | H | CH2-c-propyl | CF(CH3)2 | |
| 2.190 | H | CH2-c-butyl | CF(CH3)2 | |
| 2.191 | H | CH2-c-butyl | CF2CHF2 | |
| 2.192 | 2-F | 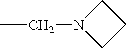 | CF(CH3)2 | |
| 2.193 | 2-F | 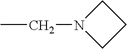 | i-propyl | |
| 2.194 | 2-F | 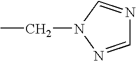 | i-propyl | |
| 2.195 | 2-F | 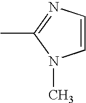 | i-propyl | |
| 2.196 | 2-Me | 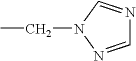 | CF(CH3)2 | |
| 2.197 | 2-Me | 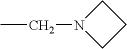 | a-propyl | |
| 2.198 | 2-Me | 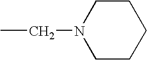 | i-propyl | |
| 2.199 | 2-Me | 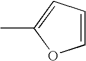 | i-propyl | |
| 2.200 | 3,4-F | 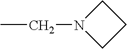 | CHFCF3 | |
| 2.201 | 3,4-F | 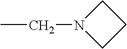 | i-propyl | |
| 2.202 | 3,4-F | 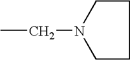 | i-propyl | |
| 2.203 | 3,4-F | 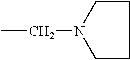 | CF(CH3)2 | |
| 2.204 | 3,4-F | 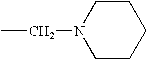 | CF2CHF2 | |
| 2.205 | 3,4-F | 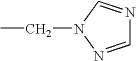 | CHFOH3 | |
| 2.206 | 3,4-F | 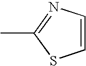 | i-propyl | |
| 2.207 | 3,4-F | 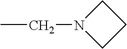 | CF(CH3)2 | |
| 2.208 | 3,5-F | 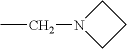 | i-propyl | |
| 2.209 | 3,5-F | 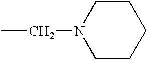 | CF(CH3)2 | |
| 2.210 | 3,5-F | 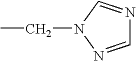 | CF2CHF2 | |
| 2.211 | 3,5-F | 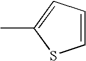 | CF2CHF2 | |
| 2.212 | 3,5-F | 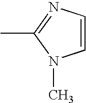 | CF(CH3)2 | |
| 2.213 | 3,5-F | 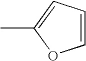 | CF(CH3)2 | |
| 2.214 | 3,5-Me | 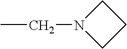 | CF(CH3)2 | |
| 2.215 | 3,5-Me | 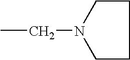 | CF(CH3)2 | |
| 2.216 | 3,5-Me | 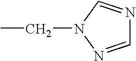 | CF2CHF2 | |
| 2.217 | 3,5-Me | 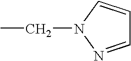 | CF2CHF2 | |
| 2.218 | 3,5-Me | 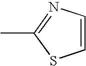 | CF2CHF2 | |
| 2.219 | 3,5-Me | 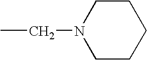 | CHFCH3 | |
| 2.220 | 3,5-Me | 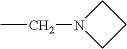 | i-propyl | |
| 2.221 | 3,5-OMe | 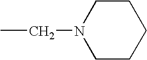 | CF(CH3)2 | |
| 2.222 | 3,5-OMe | 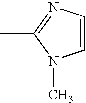 | CF3 | |
| 2.223 | 3,5-OMe | 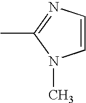 | CHFCH3 | |
| 2.224 | 3,5-OMe | 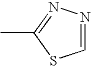 | CHFCH3 | |
| 2.225 | 3,5-OMe | 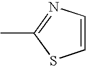 | CHFCH3 | |
| 2.226 | 3-CF3 | 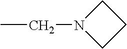 | CF3 | |
| 2.227 | 3-CF3 | 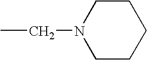 | CHFCH3 | |
| 2.228 | 3-CF3 | 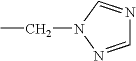 | i-propyl | |
| 2.229 | 3-CF3 | 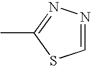 | i-propyl | |
| 2.230 | 3-CF3 | 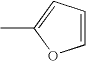 | i-propyl | |
| 2.231 | 3-Cl | 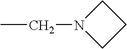 | CF(CH3)2 | |
| 2.232 | 3-Cl | 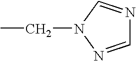 | CF(CH3)2 | |
| 2.233 | 3-Cl | 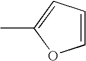 | CF(CH3)2 | |
| 2.234 | 3-Cl | 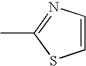 | CF2CHF2 | |
| 2.235 | 3-Cl | 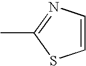 | CF(CH3)2 | |
| 2.236 | 3-Cl | 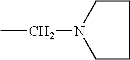 | CF(CH3)2 | |
| 2.237 | 3-Cl | 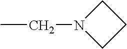 | CHFCH3 | |
| 2.238 | 3-Cl | 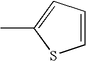 | CF3 | |
| 2.239 | 3-Cl | 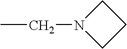 | i-propyl | |
| 2.240 | 3-Cl | 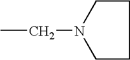 | i-propyl | |
| 2.241 | 3-Cl | 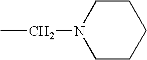 | i-propyl | |
| 2.242 | 3-Cl | 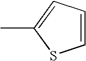 | i-propyl | |
| 2.243 | 3-F | 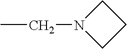 | i-propyl | |
| 2.244 | 3-F | 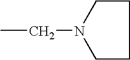 | i-propyl | |
| 2.245 | 3-F | 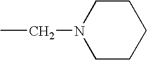 | i-propyl | |
| 2.246 | 3-F | 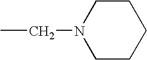 | CF(CH3)2 | |
| 2.247 | 3-F | 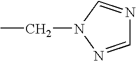 | CF3 | |
| 2.248 | 3-F | 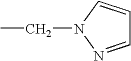 | CHFCH3 | |
| 2.249 | 3-F | 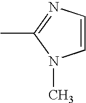 | i-propyl | oil |
| 2.250 | 3-F | 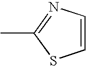 | CCl2CH3 | |
| 2.251 | 3-F | 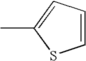 | CCl(CH3)2 | |
| 2.252 | 3-F | 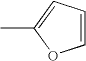 | CF(CH3)2 | |
| 2.253 | 3-F | 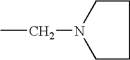 | CF(CH3)2 | |
| 2.254 | 3-F,5-Me | 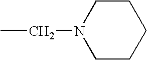 | CF(CH3)2 | |
| 2.255 | 3-F,5-Me | 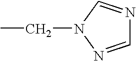 | CF(CH3)2 | |
| 2.256 | 3-F,5-Me | 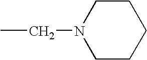 | i-propyl | |
| 2.257 | 3-F,5-OMe | 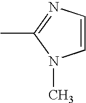 | CF(CH3)2 | |
| 2.258 | 3-F,5-OMe | 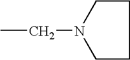 | CF2CHF2 | |
| 2.259 | 3-F,5-OMe | 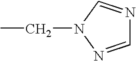 | CF(CH3)2 | |
| 2.260 | 3-Me | 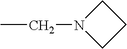 | CF(CH3)2 | |
| 2.261 | 3-Me | 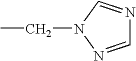 | CF(CH3)2 | |
| 2.262 | 3-Me | 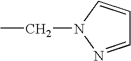 | CF2CHF2 | |
| 2.263 | 3-Me | 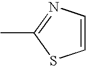 | CF2CHF2 | |
| 2.264 | 3-Me | 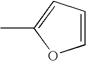 | CHFCH3 | |
| 2.265 | 3-Me | 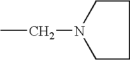 | CF3 | |
| 2.266 | 3-Me | 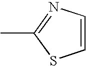 | CF3 | |
| 2.267 | 3-Me | 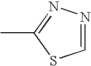 | i-propyl | |
| 2.268 | 3-Me | 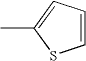 | CF(CH3)2 | |
| 2.269 | 3-Me | 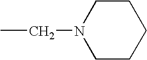 | CF(CH3)2 | |
| 2.270 | 3-Me | 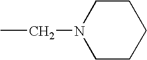 | i-propyl | |
| 2.271 | 3-Me | 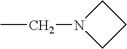 | i-propyl | |
| 2.272 | 3-Me | 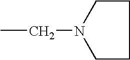 | CF(CH3)2 | |
| 2.273 | 3-Me,4-F | 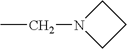 | CF(CH3)2 | |
| 2.274 | 3-Me~4-F | 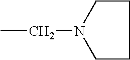 | CF(CH3)2 | |
| 2.275 | 3-Me,4-F | 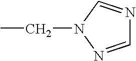 | CF(CH3)2 | |
| 2.276 | 3-Me,4-F | 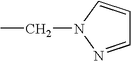 | CHFCH3 | |
| 2.277 | 3-Me,4-F | 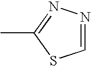 | CHFCH3 | |
| 2.278 | 3-Me,4-F | 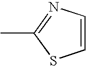 | CF2CHF2 | |
| 2.279 | 3-Me,4-F | 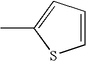 | i-propyl | |
| 2.280 | 3-Me,4-F | 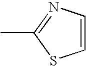 | CF(CH3)2 | |
| 2.281 | 3-Me,4-F | 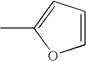 | CF(CH3)2 | |
| 2.282 | 3-Me,4-F | 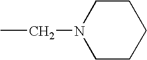 | CF(CH3)2 | |
| 2.283 | 3-Me,4-F | 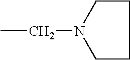 | i-propyl | |
| 2.284 | 4-Cl | 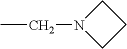 | i-propyl | |
| 2.285 | 4-Cl | 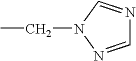 | i-propyl | |
| 2.286 | 4-F | 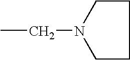 | i-propyl | |
| 2.287 | 4-F | 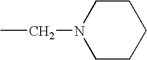 | i-propyl | |
| 2.288 | 4-F | 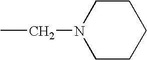 | CF(CH3)2 | |
| 2.289 | 4-F | 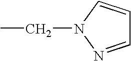 | CF2CHF2 | |
| 2.290 | 4-F | 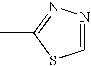 | i-propyl | |
| 2.291 | 4-F | 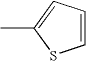 | i-propyl | |
| 2.292 | 4-F | 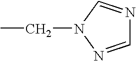 | i-propyl | |
| 2.293 | 3-Cl, 5-F | 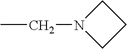 | i-propyl | |
| 2.294 | 3-Cl, 5-F | 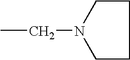 | i-propyl | |
| 2.295 | 3-Cl, 5-F | 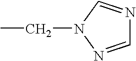 | i-propyl | |
| 2.296 | 3-Cl, 5-F | 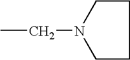 | CF(CH3)2 | |
| 2.297 | 3-Cl, 5-F | 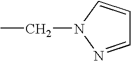 | CF2CHF2 | |
| 2.298 | 3-Cl, 5-F | 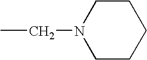 | CHFCH3 | |
| 2.299 | 3-Cl, 5-F | 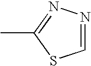 | i-propyl | |
| 2.300 | 3-Cl, 5-F | 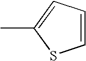 | i-propyl | |
| 2.301 | 3-Cl, 5-F | 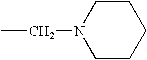 | i-propyl | |
| 2.302 | 4-F,5-CF3 | 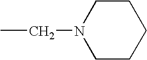 | i-propyl | |
| 2.303 | H | 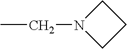 | CF(CH3)2 | |
| 2.304 | H | 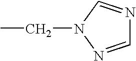 | CF(CH3)2 | oil |
| 2.305 | H | 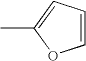 | CF(CH3)2 | |
| 2.306 | H | 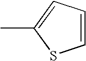 | CF(OH3)2 | |
| 2.307 | H | 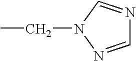 | CF2CHF2 | Oil |
| 2.308 | H | 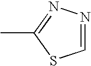 | CHFCH3 | |
| 2.309 | H | 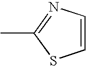 | CHFCH3 | |
| 2.310 | H | 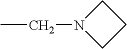 | i-propyl | |
| 2.311 | H | 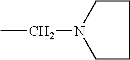 | i-propyl | |
| 2.312 | H | 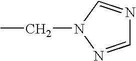 | i-propyl | oil |
| 2.313 | H | 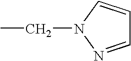 | CF(CH3)2 | |
| 2.314 | H | 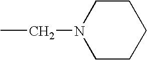 | CF(CH3)2 | |
| 2.315 | H | 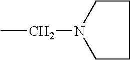 | CF(CH3)2 | |
| 2.316 | H | 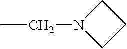 | CF2CHF2 | |

TABLE 3
Compounds of the formula (3)
(in each case the S isomer at the carbon atom which is
substituted by R², if the priority of the radicals is as follows:
1. NH, 2. R², 3. CH₂OPh(X)ₙ, 4. H)

| | |
|---|---|
| | (2) |
| 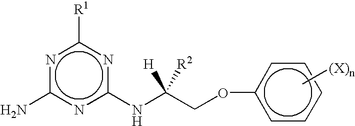 | |

| No. | (X)ₙ | R² | R¹ | Phys. data |
|---|---|---|---|---|
| 3.1 | 2,4-Cl | 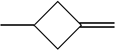 | CF3 | |
| 3.2 | 2,4-Cl | 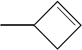 | i-propyl | |
| 3.3 | 2,4-Cl | 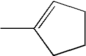 | CF(CH3)2 | |
| 3.4 | 2,4-Cl | 3,3-F2-c-butyl | i-propyl | |
| 3.5 | 2,4-F | 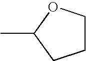 | CF2CHF2 | |
| 3.6 | 2,4-F | 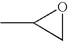 | i-propyl | |
| 3.7 | 2,4-F | CH2-c-butyl | CF(CH3)2 | |
| 3.8 | 2,4-F | 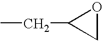 | CF(CH3)2 | |
| 3.9 | 2,5-F | 3,3-F2-c-butyl | CF(CH3)2 | |
| 3.10 | 2,5-F | 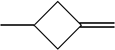 | i-propyl | |
| 3.11 | 2,5-F | 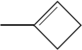 | CF(CH3)2 | |
| 3.12 | 2-Cl | 3,3-F2-c-butyl | CF2CHF2 | |
| 3.13 | 2-Cl | 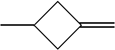 | i-propyl | |
| 3.14 | 2-Cl | 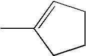 | i-propyl | |
| 3.15 | 2-Cl | 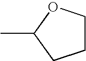 | i-propyl | |
| 3.16 | 2-Cl, 4-NO2 | 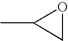 | i-propyl | |
| 3.17 | 2-Cl, 4-NO2 | 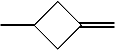 | i-propyl | |
| 3.18 | 2-F | 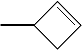 | CF(CH3)2 | |
| 3.19 | 2-F | 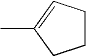 | i-propyl | |
| 3.20 | 2-F | 3,3-F2-c-butyl | i-propyl | |
| 3.21 | 2-F | 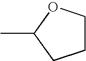 | i-propyl | |
| 3.22 | 2-Me | 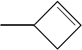 | CF(CH3)2 | |
| 3.23 | 2-Me | 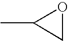 | i-propyl | |
| 3.24 | 2-Me | CH2-c-butyl | i-propyl | |
| 3.25 | 2-Me | 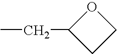 | i-propyl | |
| 3.26 | 3,4-F | 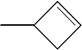 | CHFCF3 | |
| 3.27 | 3,4-F | 2,2,3,3-F4-c-butyl | i-propyl | |
| 3.28 | 3,4-F | 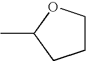 | i-propyl | |
| 3.29 | 3,4-F | c-butyl | CF(CH3)2 | crystalline |
| 3.30 | 3,4-F | c-butyl | CF2CHF2 | resin |
| 3.31 | 3,4-F | c-butyl | CHFCH3 | resin |
| 3.32 | 3,4-F | c-butyl | i-propyl | resin |
| 3.33 | 3,4-F | 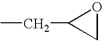 | CF(CH3)2 | |
| 3.34 | 3,5-CF3 | 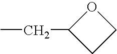 | i-propyl | |
| 3.35 | 3,5-Cl | 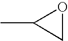 | CHFCH3 | |
| 3.36 | 3,5-Cl | 2,2,3,3-F4-c-butyl | i-propyl | |
| 3.37 | 3,5-Cl | 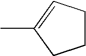 | i-propyl | |
| 3.38 | 3,5-Cl | 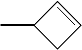 | i-propyl | |
| 3.39 | 3,5-F | c-butyl | i-propyl | resin |
| 3.40 | 3,5-F | c-butyl | CF(CH3)2 | resin |
| 3.41 | 3,5-F | 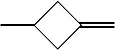 | CF2CHF2 | |
| 3.42 | 3,5-F | 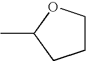 | CF2CHF2 | |
| 3.43 | 3,5-F | CH2-c-propyl | CF(CH3)2 | |
| 3.44 | 3,5-F | CH2-c-butyl | CF(CH3)2 | |
| 3.45 | 3,5-Me | c-butyl | CF(CH3)2 | resin |
| 3.46 | 3,5-Me | 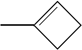 | CF(CH3)2 | |
| 3.47 | 3,5-Me | 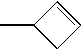 | CF2CHF2 | |
| 3.48 | 3,5-Me | 2,2,3,3-F4-c-butyl | CF2CHF2 | |
| 3.49 | 3,5-Me | 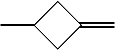 | CF2CHF2 | |
| 3.50 | 3,5-Me | c-butyl | CHFCH3 | resin |
| 3.51 | 3,5-Me | c-butyl | i-propyl | resin |
| 3.52 | 3,5-OMe | 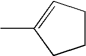 | CF(CH3)2 | |
| 3.53 | 3,5-OMe | 3,3-F2-c-butyl | CF3 | |
| 3.54 | 3,5-OMe | 2,2,3,3-F4-c-butyl | CHFCH3 | |
| 3.55 | 3,5-OMe | 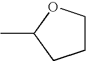 | CHFCH3 | |
| 3.56 | 3,5-OMe | 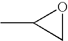 | CHFCH3 | |
| 3.57 | 3,5-OMe | 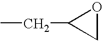 | CF(CH3)2 | |
| 3.58 | 3-CF3 | 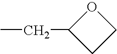 | CF3 | |
| 3.59 | 3-CF3 | 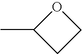 | CHFCH3 | |
| 3.60 | 3-CF3 | 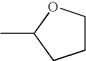 | i-propyl | |
| 3.61 | 3-CF3 | 2,2,3,3-F4-c-butyl | i-propyl | |
| 3.62 | 3-CF3 | 3,3-F2-c-butyl | i-propyl | |
| 3.63 | 3-CF3 | 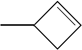 | i-propyl | |
| 3.64 | 3-Cl | c-butyl | CF(CH3)2 | resin |
| 3.65 | 3-Cl | 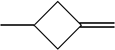 | CF(CH3)2 | |
| 3.66 | 3-Cl | 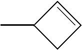 | CF(CH3)2 | |
| 3.67 | 3-Cl | 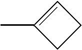 | CF(CH3)2 | |
| 3.68 | 3-Cl | c-butyl | CF2CHF2 | resin |
| 3.69 | 3-Cl | CH2-c-butyl | CF(CH3)2 | |
| 3.70 | 3-Cl | CH2-c-propyl | CF(CH3)2 | |
| 3.71 | 3-Cl | c-butyl | CHFCH3 | resin |
| 3.72 | 3-Cl | 3,3-F2-c-butyl | CF3 | |
| 3.73 | 3-Cl | c-butyl | i-propyl | resin |
| 3.74 | 3-Cl | 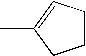 | i-propyl | |
| 3.75 | 3-Cl | 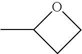 | i-propyl | |
| 3.76 | 3-Cl | 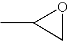 | i-propyl | |
| 3.77 | 3-Cl, 5-Me | c-butyl | CF2CHF2 | |
| 3.78 | 3-Cl, 5-Me | 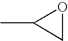 | CF3 | |
| 3.79 | 3-Cl, 5-Me | 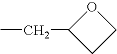 | CHFCH3 | |
| 3.80 | 3-F | 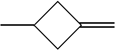 | i-propyl | |
| 3.81 | 3-F | 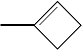 | i-propyl | |
| 3.82 | 3-F | 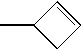 | i-propyl | |
| 3.83 | 3-F | c-butyl | CF(CH3)2 | resin |
| 3.84 | 3-F | 3,3-F2-c-butyl | CF3 | |
| 3.85 | 3-F | c-butyl | CHFCH3 | resin |
| 3.86 | 3-F | c-butyl | i-propyl | resin |
| 3.87 | 3-F | c-butyl | CCl2CH3 | resin |
| 3.88 | 3-F | c-butyl | CCl(CH3)2 | resin |
| 3.89 | 3-F | CH2-c-propyl | CF(CH3)2 | |
| 3.90 | 3-F | CH2-c-butyl | CF(CH3)2 | |
| 3.91 | 3-F, 5-Me | 3,3-F2-c-butyl | CF(CH3)2 | |
| 3.92 | 3-F, 5-Me | 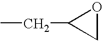 | CF(CH3)2 | |
| 3.93 | 3-F, 5-Me | 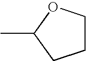 | CHFCH3 | |
| 3.94 | 3-F, 5-Me | CH2-c-pentyl | i-propyl | |
| 3.95 | 3-F, 5-Me | 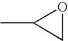 | i-propyl | |
| 3.96 | 3-F, 5-OMe | 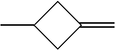 | CF(CH3)2 | |
| 3.97 | 3-F, 5-OMe | 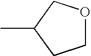 | CF2CHF2 | |
| 3.98 | 3-F, 5-OMe | 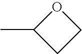 | CF2CHF2 | |
| 3.99 | 3-F, 5-OMe | 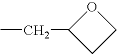 | CF(CH3)2 | |
| 3.100 | 3-Me | c-butyl | CF(CH3)2 | resin |
| 3.101 | 3-Me | 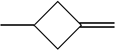 | CF(CH3)2 | |
| 3.102 | 3-Me | c-butyl | CF2CHF2 | resin |
| 3.103 | 3-Me | 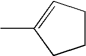 | CF2CHF2 | |
| 3.104 | 3-Me | c-butyl | CHFCH3 | resin |
| 3.105 | 3-Me | CH2-c-butyl | CF(CH3)2 | |
| 3.106 | 3-Me | 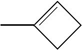 | CF3 | |
| 3.107 | 3-Me | 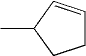 | CF3 | |
| 3.108 | 3-Me | c-butyl | i-propyl | resin |
| 3.109 | 3-Me | c-pentyl | CF(CH3)2 | crystalline |
| 3.110 | 3-Me | c-pentyl | CF2CHF2 | crystalline |
| 3.111 | 3-Me | 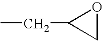 | CF(CH3)2 | |
| 3.112 | 3-Me | c-pentyl | i-propyl | resin |
| 3.113 | 3-Me | c-hexyl | i-propyl | resin |
| 3.114 | 3-Me | c-hexyl | CF(CH3)2 | crystalline |
| 3.115 | 3-Me, 4-F | c-butyl | CF(CH3)2 | resin |
| 3.116 | 3-Me, 4-F | 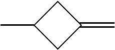 | CF(CH3)2 | |
| 3.117 | 3-Me, 4-F | 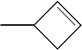 | CF(CH3)2 | |
| 3.118 | 3-Me, 4-F | c-butyl | CHFCH3 | resin |
| 3.119 | 3-Me, 4-F | 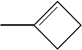 | CHFCH3 | |
| 3.120 | 3-Me, 4-F | c-butyl | CF2CHF2 | resin |
| 3.121 | 3-Me, 4-F | c-butyl | i-propyl | resin |
| 3.122 | 3-Me, 4-F | CH2-c-butyl | CF(CH3)2 | |
| 3.123 | 3-Me, 4-F | CH2-c-propyl | CF(CH3)2 | |
| 3.124 | 3-Me, 4-F | 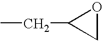 | CF(CH3)2 | |
| 3.125 | 3-Me, 4-F | 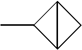 | i-propyl | |
| 3.126 | 3-OEt | 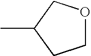 | CF(CH3)2 | |
| 3.127 | 3-OEt | 3,3-(CH3)2-c-butyl | CF(CH3)2 | |
| 3.128 | 3-OEt | 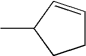 | CF3 | |
| 3.129 | 3-OEt | 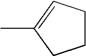 | i-propyl | |
| 3.130 | 3-OMe | c-butyl | CF(CH3)2 | resin |
| 3.131 | 3-OMe | 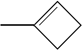 | CF3 | |
| 3.132 | 3-OMe | c-butyl | CHFCH3 | resin |
| 3.133 | 3-OMe | c-butyl | i-propyl | resin |
| 3.134 | 3-OMe | c-butyl | CCl2CH3 | resin |
| 3.135 | 3-OMe | c-butyl | CCl(CH3)2 | resin |
| 3.136 | 3-OMe, 5-Cl | 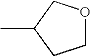 | CHFCH3 | |
| 3.137 | 3-OMe, 5-Cl | 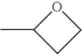 | CHFCH3 | |
| 3.138 | 3-OMe, 5-Cl | CH2-c-pentyl | CHFCH3 | |
| 3.139 | 4-Br | 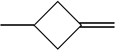 | CF(CH3)2 | |
| 3.140 | 4-Br | 3,3-(CH3)2-c-butyl | CF2CHF2 | |
| 3.141 | 4-Br | 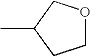 | CHFCH3 | |
| 3.142 | 4-Br | 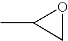 | i-propyl | |
| 3.143 | 4-Cl | 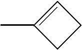 | CF(CH3)2 | |
| 3.144 | 4-Cl | 3,3-(CH3)2-c-butyl | CF2CHF2 | |
| 3.145 | 4-Cl | 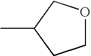 | CHFCH3 | |
| 3.146 | 4-Cl | 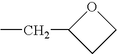 | i-propyl | |
| 3.147 | 4-Cl | 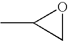 | i-propyl | |
| 3.148 | 4-Cl | 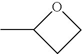 | i-propyl | |
| 3.149 | 4-Cl | 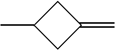 | i-propyl | |
| 3.150 | 4-F | 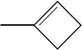 | i-propyl | |
| 3.151 | 4-F | 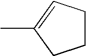 | i-propyl | |
| 3.152 | 4-F | c-butyl | CF(CH3)2 | crystalline |
| 3.153 | 4-F | c-butyl | CF2CHF2 | |
| 3.154 | 4-F | c-butyl | CHFCH3 | resin |
| 3.155 | 4-F | c-butyl | i-propyl | resin |
| 3.156 | 4-F | 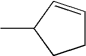 | i-propyl | |
| 3.157 | 4-F | 3,3-(CH3)2-c-butyl | i-propyl | |
| 3.158 | 3-Cl, 5-F | 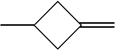 | i-propyl | |
| 3.159 | 3-Cl, 5-F | 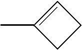 | i-propyl | |
| 3.160 | 3-Cl, 5-F | 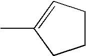 | i-propyl | |
| 3.161 | 3-Cl, 5-F | c-butyl | CF(CH3)2 | |
| 3.162 | 3-Cl, 5-F | c-butyl | CF2CHF2 | |
| 3.163 | 3-Cl, 5-F | c-butyl | CHFCH3 | |
| 3.164 | 3-Cl, 5-F | c-butyl | i-propyl | |
| 3.165 | 3-Cl, 5-F | 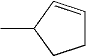 | i-propyl | |
| 3.166 | 3-Cl, 5-F | 3,3-(CH3)2-c-butyl | i-propyl | |
| 3.167 | 4-F, 5-CF3 | 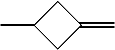 | i-propyl | |
| 3.168 | 4-Me | CH2-c-pentyl | CF(CH3)2 | |
| 3.169 | 4-Me | 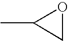 | i-propyl | |
| 3.170 | 4-Me | 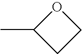 | i-propyl | |
| 3.171 | 4-Me | 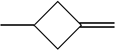 | i-propyl | |
| 3.172 | 4-Me | 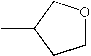 | i-propyl | |
| 3.173 | 4-Me | 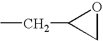 | CF(CH3)2 | |
| 3.174 | 4-OCF3 | 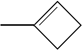 | CF(CH3)2 | |
| 3.175 | 4-OCF3 | 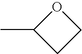 | i-propyl | |
| 3.176 | 4-OCF3 | 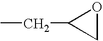 | CF(CH3)2 | |
| 3.177 | 4-OCF3 | 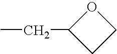 | i-propyl | |
| 3.178 | H | c-butyl | CF(CH3)2 | resin |
| 3.179 | H | 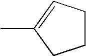 | CF(CH3)2 | |
| 3.180 | H | 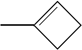 | CF(CH3)2 | |
| 3.181 | H | 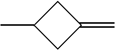 | CF(CH3)2 | |
| 3.182 | H | c-butyl | CF2CHF2 | resin |
| 3.183 | H | c-butyl | CHFCH3 | resin |
| 3.184 | H | 3,3-(CH3)2-c-butyl | CHFCH3 | |
| 3.185 | H | c-butyl | i-propyl | resin |
| 3.186 | H | 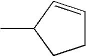 | i-propyl | |
| 3.187 | H | 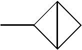 | i-propyl | |
| 3.188 | H | 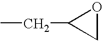 | CF(CH3)2 | |
| 3.189 | H | CH2-c-propyl | CF(CH3)2 | |
| 3.190 | H | CH2-c-butyl | CF(CH3)2 | |
| 3.191 | H | CH2-c-butyl | CF2CHF2 | |
| 3.192 | 2-F | 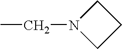 | CF(CH3)2 | |
| 3.193 | 2-F | 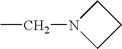 | i-propyl | |
| 3.194 | 2-F | 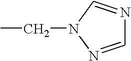 | i-propyl | |
| 3.195 | 2-F | 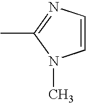 | i-propyl | |
| 3.196 | 2-Me | 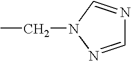 | CF(CH3)2 | |
| 3.197 | 2-Me | 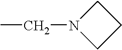 | i-propyl | |
| 3.198 | 2-Me | 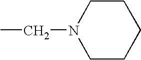 | i-propyl | |
| 3.199 | 2-Me | 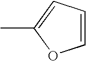 | i-propyl | |
| 3.200 | 3,4-F | 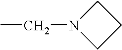 | CHFCF3 | |
| 3.201 | 3,4-F | 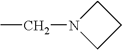 | i-propyl | |
| 3.202 | 3,4-F | 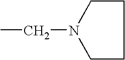 | i-propyl | |
| 3.203 | 3,4-F | 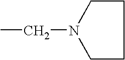 | CF(CH3)2 | |
| 3.204 | 3,4-F | 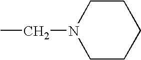 | CF2CHF2 | |
| 3.205 | 3,4-F | 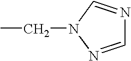 | CHFCH3 | |
| 3.206 | 3,4-F | 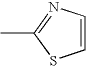 | i-propyl | |
| 3.207 | 3,4-F | 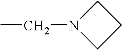 | CF(CH3)2 | |
| 3.208 | 3,5-F | 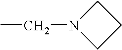 | i-propyl | |
| 3.209 | 3,5-F | 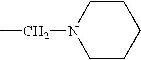 | CF(CH3)2 | |
| 3.210 | 3,5-F | 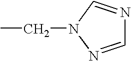 | CF2CHF2 | |
| 3.211 | 3,5-F | 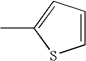 | CF2CHF2 | |
| 3.212 | 3,5-F | 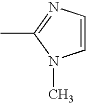 | CF(CH3)2 | |
| 3.213 | 3,5-F | 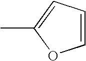 | CF(CH3)2 | |
| 3.214 | 3,5-Me | 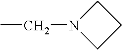 | CF(CH3)2 | |
| 3.215 | 3,5-Me | 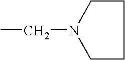 | CF(CH3)2 | |
| 3.216 | 3,5-Me | 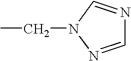 | CF2CHF2 | |
| 3.217 | 3,5-Me | 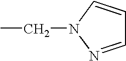 | CF2CHF2 | |
| 3218 | 3,5-Me | 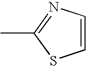 | CF2CHF2 | |
| 3.219 | 3,5-Me | 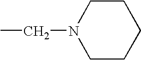 | CHFCH3 | |
| 3.220 | 3,5-Me | 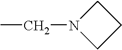 | i-propyl | |
| 3.221 | 3,5-OMe | 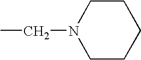 | CF(CH3)2 | |
| 3.222 | 3,5-OMe | 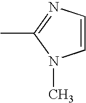 | CF3 | |
| 3.223 | 3,5-OMe | 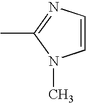 | CHFCH3 | |
| 3.224 | 3,5-OMe | 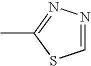 | CHFCH3 | |
| 3.225 | 3,5-OMe | 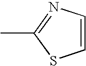 | CHFCH3 | |
| 3.226 | 3-CF3 | 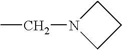 | CF3 | |
| 3.227 | 3-CF3 | 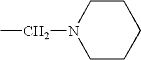 | CHFCH3 | |
| 3.228 | 3-CF3 | 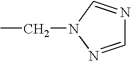 | i-propyl | |
| 3.229 | 3-CF3 | 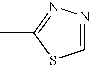 | i-propyl | |
| 3.230 | 3-CF3 | 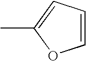 | i-propyl | |
| 3.231 | 3-Cl | 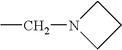 | CF(CH3)2 | |
| 3.232 | 3-Cl | 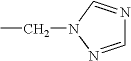 | CF(CH3)2 | |
| 3.233 | 3-Cl | 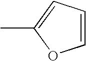 | CF(CH3)2 | |
| 3.234 | 3-Cl | 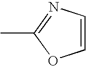 | CF2CHF2 | |
| 3.235 | 3-Cl | 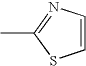 | CF(CH3)2 | |
| 3.236 | 3-Cl | 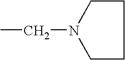 | CF(CH3)2 | |
| 3.237 | 3-Cl | 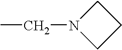 | CHFCH3 | |
| 3.238 | 3-Cl | 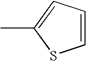 | CF3 | |
| 3.239 | 3-Cl | 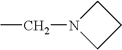 | i-propyl | |
| 3.240 | 3-Cl | 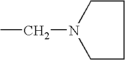 | i-propyl | |
| 3.241 | 3-Cl | 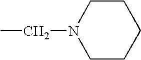 | i-propyl | |
| 3.242 | 3-Cl | 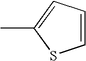 | i-propyl | |
| 3.243 | 3-F | 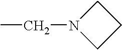 | i-propyl | |
| 3.244 | 3-F | 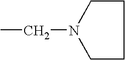 | i-propyl | |
| 3.245 | 3-F | 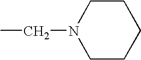 | i-propyl | |
| 3.246 | 3-F | 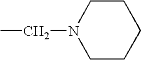 | CF(CH3)2 | |
| 3.247 | 3-F | 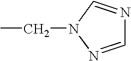 | CF3 | |
| 3.248 | 3-F | 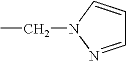 | CHFCH3 | |
| 3.249 | 3-F | 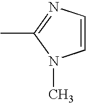 | i-propyl | oil |
| 3.250 | 3-F | 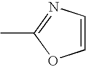 | CCl2CH3 | |
| 3.251 | 3-F | 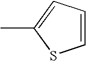 | CCl(CH3)2 | |
| 3.252 | 3-F | 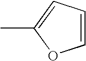 | CF(CH3)2 | |
| 3.253 | 3-F | 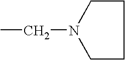 | CF(CH3)2 | |
| 3.254 | 3-F, 5-Me | 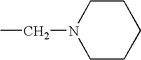 | CF(CH3)2 | |
| 3.255 | 3-F, 5-Me | 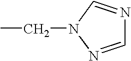 | CF(CH3)2 | |
| 3.256 | 3-F, 5-Me | 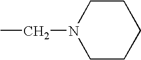 | i-propyl | |
| 3.257 | 3-F, 5-OMe | 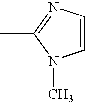 | CF(CH3)2 | |
| 3.258 | 3-F, 5-OMe | 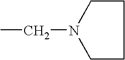 | CF2CHF2 | |
| 3.259 | 3-F, 5-OMe | 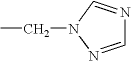 | CF(CH3)2 | |
| 3.260 | 3-Me | 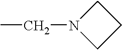 | CF(CH3)2 | |
| 3.261 | 3-Me | 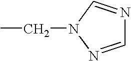 | CF(CH3)2 | |
| 3.262 | 3-Me | 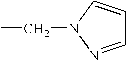 | CF2CHF2 | |
| 3.263 | 3-Me | 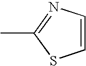 | CF2CHF2 | |
| 3.264 | 3-Me | 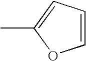 | CHFCH3 | |
| 3.265 | 3-Me | 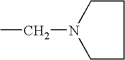 | CF3 | |
| 3.266 | 3-Me | 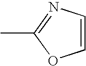 | CF3 | |
| 3.267 | 3-Me | 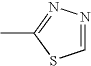 | i-propyl | |
| 3.268 | 3-Me | 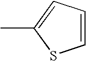 | CF(CH3)2 | |
| 3.269 | 3-Me | 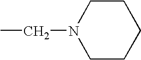 | CF(CH3)2 | |
| 3.270 | 3-Me | 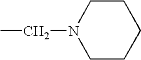 | i-propyl | |
| 3.271 | 3-Me | 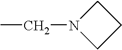 | i-propyl | |
| 3.272 | 3-Me | 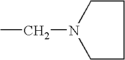 | CF(CH3)2 | |
| 3.273 | 3-Me, 4-F | 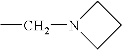 | CF(CH3)2 | |
| 3.274 | 3-Me, 4-F | 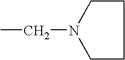 | CF(CH3)2 | |
| 3.275 | 3-Me, 4-F | 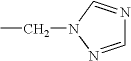 | CF(CH3)2 | |
| 3.276 | 3-Me, 4-F | 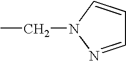 | CHFCH3 | |
| 3.277 | 3-Me, 4-F | 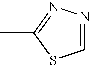 | CHFCH3 | |
| 3.278 | 3-Me, 4-F | 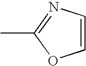 | CF2CHF2 | |
| 3.279 | 3-Me, 4-F | 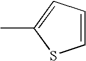 | i-propyl | |
| 3.280 | 3-Me, 4-F | 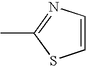 | CF(CH3)2 | |
| 3.281 | 3-Me, 4-F | 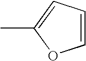 | CF(CH3)2 | |
| 3.282 | 3-Me, 4-F | 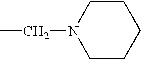 | CF(CH3)2 | |
| 3.283 | 3-Me, 4-F | 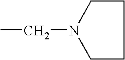 | i-propyl | |
| 3.284 | 4-Cl | 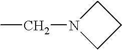 | i-propyl | |
| 3.285 | 4-Cl | 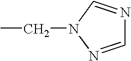 | i-propyl | |
| 3.286 | 4-F | 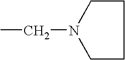 | i-propyl | |
| 3.287 | 4-F | 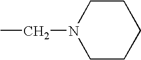 | i-propyl | |
| 3.288 | 4-F | 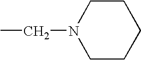 | CF(CH3)2 | |
| 3.289 | 4-F | 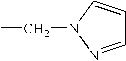 | CF2CHF2 | |
| 3.290 | 4-F | 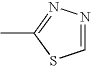 | i-propyl | |
| 3.291 | 4-F | 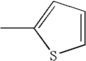 | i-propyl | |
| 3.292 | 4-F | 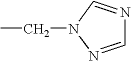 | i-propyl | |
| 3.293 | 3-Cl, 5-F | 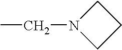 | i-propyl | |
| 3.294 | 3-Cl, 5-F | 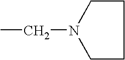 | i-propyl | |
| 3.295 | 3-Cl, 5-F | 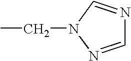 | i-propyl | |
| 3.296 | 3-Cl, 5-F | 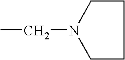 | CF(CH3)2 | |
| 3.297 | 3-Cl, 5-F | 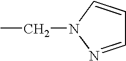 | CF2CHF2 | |
| 3.298 | 3-Cl, 5-F | 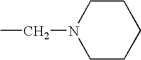 | CHFCH3 | |
| 3.299 | 3-Cl, 5-F | 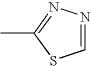 | i-propyl | |
| 3.300 | 3-Cl, 5-F | 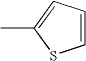 | i-propyl | |
| 3.301 | 3-Cl, 5-F | 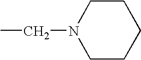 | i-propyl | |
| 3.302 | 4-F, 5-CF3 | 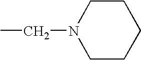 | i-propyl | |
| 3.303 | H | 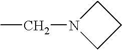 | CF(CH3)2 | |
| 3.304 | H | 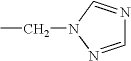 | CF(CH3)2 | oil |
| 3.305 | H | 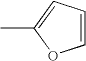 | CF(CH3)2 | |
| 3.306 | H | 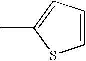 | CF(CH3)2 | |
| 3.307 | H | 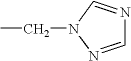 | CF2CHF2 | oil |
| 3.308 | H | 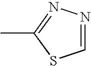 | CHFCH3 | |
| 3.309 | H | 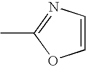 | CHFCH3 | |
| 3.310 | H | 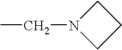 | i-propyl | |
| 3.311 | H | 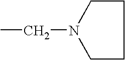 | i-propyl | |
| 3.312 | H | 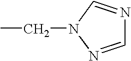 | i-propyl | oil |
| 3.313 | H | 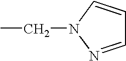 | CF(CH3)2 | |
| 3.314 | H | 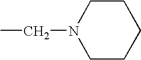 | CF(CH3)2 | |
| 3.315 | H | 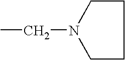 | CF(CH3)2 | |
| 3.316 | H | 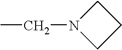 | CF2CHF2 | |

### B. FORMULATION EXAMPLES

a) A dust is obtained by mixing 10 parts by weight of a compound of the formula (I) and 90 parts by weight of talc as inert substance and comminuting the mixture in a hammer mill.

b) A wettable powder which is readily dispersible in water is obtained by mixing 25 parts by weight of a compound of the formula (I), 64 parts by weight of kaolin-containing quartz as inert substance, 10 parts by weight of potassium lignosulfonate and 1 part by weight of sodium oleoylmethyltaurinate as wetter and dispersant and grinding the mixture in a pinned-disk mill.

c) A dispersion concentrate which is readily dispersible in water is obtained by mixing 20 parts by weight of a compound of the formula (I) with 6 parts by weight of alkylphenol polyglycol ether (®Triton X 207), 3 parts by weight of isotridecanol polyglycol ether (8 EO) and 71 parts by weight of paraffinic mineral oil (boiling range for example approx. 255 to above 277° C.) and grinding the mixture in a ball mill to a fineness of below 5 microns.

d) An emulsifiable concentrate is obtained from 15 parts by weight of a compound of the formula (I), 75 parts by weight of cyclohexanone as the solvent and 10 parts by weight of ethoxylated nonylphenol as the emulsifier.

e) Water-dispersible granules are obtained by mixing

75 parts by weight of a compound of the formula (I),

10 parts by weight of calcium lignosulfonate,

5 parts by weight of sodium lauryl sulfate,

3 parts by weight of polyvinyl alcohol and

7 parts by weight of kaolin

grinding the mixture on a pinned-disk mill and granulating the powder in a fluidized bed by spraying on water as the granulation liquid.

f) Water-dispersible granules are also obtained by homogenizing and precomminuting, on a colloid mill,

25 parts by weight of a compound of the formula (I),

5 parts by weight of sodium 2,2′-dinaphthylmethane-6,6′-disulfonate

2 parts by weight of sodium oleoylmethyltaurinate,

1 part by weight of polyvinyl alcohol,

17 parts by weight of calcium carbonate and

50 parts by weight of water,

subsequently grinding the mixture in a bead mill and atomizing and drying the resulting suspension in a spray tower by means of a single-substance nozzle.

### C. BIOLOGICAL EXAMPLES

1. Pre-Emergence Effect on Weeds

Seeds or rhizome pieces of monocotyledonous and dicotyledonous weed plants are placed in sandy loam soil in plastic pots and covered with soil. The compounds according to the invention which are formulated in the form of wettable powders or emulsion concentrates were then applied to the surface of the soil cover in the form of aqueous suspensions or emulsions at an application rate of 600 to 800 l of water/ha (converted), in various dosages.

After the treatment, the pots are placed in a greenhouse and kept under good growth conditions for the weeds. After the test plants had emerged, the damage to the plants or the negative effect on the emergence was scored visually after a test period of 3 to 4 weeks by comparison with untreated controls. As shown by the test results, compounds according to the invention have good herbicidal pre-emergence activity against a broad spectrum of weed grasses and broad-leafed weeds. For example, in the test the compounds of Example Nos. 29, 30, 31, 32, 39, 40, 45, 50, 51, 64, 68, 71, 73, 83, 85, 86, 87, 88, 100, 102, 104,108, 109, 110, 112, 113, 118, 120, 121, 130, 132, 133, 134, 135, 152, 154, 155, 178, 182, 183, 185, 249, 304, 307 and 312 (see Table 1) have very good herbicidal activity pre-emergence against harmful plants such as Stellaria media, Lolium multiflorum, Amaranthus retroflexus, Sinapis arvensis, Avena fatua and Setaria viridis at an application rate of 1 kg and less of active substance per hectare.

2. Post-Emergence Effect on Weeds

Seeds or rhizome pieces of monocotyledonous and dicotyledonous weeds were placed in sandy loam soil in plastic pots, covered with soil and grown in a greenhouse under good growth conditions. Three weeks after sowing, the test plants were treated at the three-leaf stage. The compounds according to the invention which were formulated as wettable powders or emulsion concentrates were sprayed, at various dosages, onto the, green parts of the plants at an application rate of 600 to 800 l of water/ha (converted). After the test plants had remained in the greenhouse for about 3 to 4 weeks under optimum growth conditions, the effect of the preparations was scored visually by comparison with untreated controls. The agents according to the invention also have good herbicidal activity post-emergence against a broad spectrum of economically important weed grasses and broad-leafed weeds. For example, the compounds of Example Nos. 29, 30, 31, 32, 39, 40, 45, 50, 51, 64, 68, 71, 73, 83, 85, 86, 87, 88, 100, 102, 104,108, 109, 110, 112, 113,118, 120, 121, 130, 132, 133, 134, 135, 152, 154, 155, 178, 182, 183, 185, 249, 304, 307 and 312 (see Table 1) have very good herbicidal activity post-emergence against harmful plants such as Stellaria media, Lolium multiflorum, Amaranthus retroflexus, Sinapis arvensis, Avena fatua und Setaria viridis at an application rate of 1 kg and less of active substance per hectare.

3. Action on Harmful Plants in Rice

Transplanted and sown rice and also typical rice weeds (gramineous and broad-leaved) are cultivated in closed plastic pots in a greenhouse to the three-leaf stage (Echinochloa crus-galli 1.5-leaf) under paddy rice conditions (dammed height of water: 2-3 cm). This is followed by treatment with the novel compounds. For this purpose, the formulated active compounds are suspended, dissolved or emulsified in water and applied by pouring them into the dammed water around the test plants in different dosages. After this treatment, the test plants are set up in a greenhouse under optimum growth conditions and are maintained in this way throughout the test period.

About three weeks after application, evaluation is made by visual scoring of the damage to the plants by comparison with untreated controls. The compounds according to the invention show very good herbicidal activity against harmful plants. For example, the compounds of Example Nos. 29, 30, 31, 32, 39, 40, 45, 50, 51, 64, 68, 71, 73, 83, 85, 86, 87, 88, 100, 102, 104, 108, 109, 110, 112, 113, 118, 120, 121, 130, 132, 133, 134, 135, 152, 154, 155, 178, 182, 183, 185, 249, 304, 307 and 312 (see Table 1) show very good herbicidal activity in the test against harmful plants which are typical for rice crops, for example Cyperus iria and Echinochloa crus-galli.

4. Tolerance by Crop Plants

In further greenhouse experiments, seeds of a substantial number of crop plants and weeds are placed in sandy loam soil and covered with soil. Some of the pots were treated immediately as described under Section 1, and the remaining pots are placed in the greenhouse until the plants have developed two to three true leaves and then sprayed with various dosages of the substances of the formula (I) according to the invention, as described under Section 2. Visual scoring four to five weeks after the application and after the plants had been in the greenhouse reveals that compounds according to the invention left dicotyledonous crops such as soya, cotton, oilseed rape, sugar beet or potatoes unharmed even when high dosages of active ingredient were used by the pre- and post-emergence method. Moreover, some substances also spare Gramineae crops such as barley, wheat, rye, sorghum species, corn or rice. Some of the compounds of the formula (I) have high selectivity, and they are therefore suitable for controlling undesirable vegetation in agricultural crops.

1. A compound of the formula (I) or a salt thereof 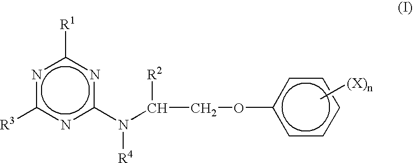
in which
R¹ is aryl, which is unsubstituted or substituted, or (C₃-C₉)cycloalkyl, which is unsubstituted or substituted, or heterocyclyl, which is substituted or unsubstituted, or
is (C₁-C₆)alkyl, (C₂-C₆)alkenyl or (C₂-C₆)alkynyl,
where each of the 3 lastmentioned radicals is unsubstituted or substituted by one or more radicals selected from the group consisting of halogen, hydroxyl, cyano, nitro, thiocyanato, (C₁-C₄)alkoxy, (C₁-C₄)haloalkoxy, (C₂-C₄)alkenyloxy, (C₂-C₄)haloalkenyloxy, (C₁-C₄)alkylthio, (C₁-C₄)alkylsulfinyl, (C₁-C₄)alkylsulfonyl, (C₁-C₄)haloalkylsulfinyl, (C₁-C₄)haloalkylsulfonyl and (C₃-C₉)cycloalkyl, which is unsubstituted or substituted, and phenyl, which is unsubstituted or substituted, and heterocyclyl, which is unsubstituted or substituted, and radicals of the formulae R′—C(═Z′)—, R′—C(═Z′)—Z—, R′—Z—C(═Z′)—, R′R″N—C(═Z′)—, R′—Z—C(═Z′)—O—, R′R″N—C(═Z′)—Z—, R′—Z—C(═Z′)—NR″— and R′R″N—C(═Z′)—NR′″—, in which
R′, R″ and R′″, in each case independently of one another, are (C₁-C₆)alkyl, aryl, aryl-(C₁-C₆)alkyl, (C₃-C₉)cycloalkyl or (C₃-C₉)cycloalkyl-(C₁-C₆)alkyl, where each of the 5 lastmentioried radicals is unsubstituted or substituted, and where Z and Z′ independently of one another are each an oxygen or sulfur atom,
R² is a carbocyclic saturated, partially unsaturated or aromatic ring having 4 to 6 ring atoms or a heteroaromatic, saturated or partially unsaturated heterocyclic radical, which contains 3 to 6 ring atoms and 1 to 4 heteroatoms selected from the group consisting of N, O and S in the heterocyclic ring, where each of the cyclic radicals mentioned is unsubstituted or substituted, or
is a radical of the formula —A—Z, in which
A is straight-chain alkylene having 1 to 4 carbon atoms or straight-chain alkenylene or alkynylene having in each case 2 to 5 carbon atoms, where each of the three lastmentioned diradicals is unsubstituted or substituted by one or more radicals selected from the group consisting of halogen, nitro, cyano, thiocyanato, (C₁-C₄)alkyl, (C₁-C₄)alkoxy, (C₁-C₄)alkylthio, (C₁-C₄)haloalkyl and (C₁-C₄)haloalkoxy, and
Z is a carbocyclic saturated, partially unsaturated or aromatic radical having 3 to 6 ring atoms or is a heteroaromatic, saturated or partially unsaturated heterocyclic ring which contains 3 to 6 ring atoms and 1 to 4 heteroatoms selected from the group consisting of N, O or S in the heterocyclic ring, where each of the cyclic radicals mentioned is unsubstituted or substituted,
R³ is hydrogen, (C₁-C₆)alkyl, aryl or (C₃-C₉)cycloalkyl, where each of the 3 lastmentioned radicals is unsubstituted or substituted, or is a radical of the formula —N(B¹—D¹)(B²—D²) or —NR′—N(B¹—D¹)(B²—D²), where B¹, B², D¹ and D² are each as defined below and R′=hydrogen, (C₁-C₆)alkyl or [(C₁-C₄)alkyl]carbonyl,
R⁴ is a radical of the formula, —B³—D³, where B³ and D³ are as defined below,
B¹, B² and B³, in each case independently of one another, are a direct bond or a divalent group of the formula —C(═Z*)—, —C(═Z*)—Z**—, —C(═Z*)—NH—or —C(═Z*)—NR*, where Z*=an oxygen or sulfur atom, Z**=an oxygen or sulfur atom and R*=(C₁-C₆)alkyl, aryl, aryl-(C₁-C₆)alkyl, (C₃-C₉)cycloalkyl or (C₃-C₉)cycloalkyl-(C₁-C₆)alkyl, where each of the 5 lastmentioned radicals is unsubstituted or substituted,
D¹, D², D³, in each case independently of one another, are hydrogen, (C₁-C₆)alkyl, aryl, aryl-(C₁-C₆)alkyl, (C₃-C₉)cycloalkyl or (C₃-C₉)cycloalkyl-(C₁-C₆)alkyl, where each of the 5 lastmentioned radicals is unsubstituted or substituted,
(X)ₙ are n substituents X, where X, in each case independently of the others, is halogen, hydroxyl, amino, nitro, formyl, carboxyl, cyano, thiocyanato, aminocarbonyl or (C₁-C₆)alkyl, (C₁-C₆)alkoxy, (C₁-C₆)alkylthio, mono(C₁-C₆)alkylamino, di(C₁-C₄)alkylamino, (C₂-C₆)alkenyl, (C₂-C₆)alkynyl, [(C₁-C₆)alkyl]carbonyl, [(C₁-C₆)alkoxy]carbonyl, mono(C₁-C₆)alkylaminocarbonyl, di(C₁-C₄)alkylaminocarbonyl, N-(C₁-C₆)alkanoylamino or N-(C₁-C₄)alkanoyl-N-(C₁-C₄)alkylamino,
where each of the 13 lastmentioned radicals is unsubstituted or substituted,
or is (C₃-C₉)cycloalkyl, (C₃-C₉)cycloalkoxy, (C₃-C₉)cycloalkylamino, phenyl, phenoxy, phenylthio, phenylcarbonyl, heterocyclyl, heterocyclyloxy, heterocyclylthio or heterocyclylamino,
where each of the 11 lastmentioned radicals is unsubstituted or substituted,
or two adjacent radicals X together are a fused-on cycle having 4 to 6 ring atoms which is carbocyclic or contains hetero ring atoms selected from the group consisting of O, S and N and which is unsubstituted or substituted by one or more radicals selected from the group consisting of halogen, (C₁-C₄)alkyl and oxo,
n is 0, 1, 2, 3,4 or 5, and
heterocyclyl in the radicals mentioned above independently of the others is in each case a heterocyclic radical having 3 to 7 ring atoms and 1 to 4 heteroatoms selected from the group consisting of N, O and S.

2. A compound or a salt thereof as claimed in claim 1, wherein
R′ is phenyl, which is unsubstituted or substituted by one or more radicals selected from the group consisting of halogen, hydroxyl, amino, nitro, formyl, carboxyl, sulfo, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylamino-carbonyl, di(C₁-C₄)alkylaminocarbonyl, (C₁-C₄)alkylsulfonyl and (C₁-C₄)haloalkylsulfonyl and, including substituents, has 6 to 30 carbon atoms, or
is (C₃-C₉)cycloalkyl, which is unsubstituted or substituted by one or more radicals selected from the group consisting of halogen, hydroxyl, amino, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino and di(C₁-C₄)-alkylamino and, including substituents, has 3 to 30 carbon atoms, or
is heterocyclyl, which is unsubstituted or substituted by one or more radicals selected from the group consisting of halogen, hydroxyl, amino, nitro, formyl, carboxyl, sulfonyl, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylamino-carbonyl, di(C₁-C₄)alkylaminocarbonyl, (C₁-C₄)alkylsulfonyl and (C₁-C₄)haloalkylsulfonyl and, including substituents, has 2 to 30 carbon atoms, or
is (C₁-C₆)alkyl, (C₂-C₆)alkenyl or (C₂-C₆)alkynyl,
where each of the 3 lastmentioned radicals is unsubstituted or substituted by one or more radicals selected from the group consisting of halogen, hydroxyl, cyano, nitro, thiocyanato, (C₁-C₄)alkoxy, (C₁-C₄)haloalkoxy, (C₂-C₄)alkenyloxy, (C₂-C₄)haloalkenyloxy, (C₁-C₄)alkylthio, (C₁-C₄)alkylsulfinyl, (C₁-C₄)alkyl-sulfonyl, (C₁-C₄)haloalkylsulfinyl, (C₁-C₄)haloalkylsulfonyl and (C₃-C₆)cycloalkyl, which is unsubstituted or substituted by one or more radicals selected from the group consisting of halogen, hydroxyl, amino, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino and di(C₁-C₄)alkylamino, and phenyl and heterocyclyl, where each of the two lastmentioned radicals is unsubstituted or substituted by one or more radicals selected from the group consisting of halogen, hydroxyl, amino, nitro, formyl, carboxyl, sulfonyl, cyano, thiocyanato, (C₁-C₄)alkyl, (C₁-C₄)haloalkyl, (C₁-C₄)alkoxy, (C₁-C₄)haloalkoxy, (C₁-C₄)alkyl-thio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl and (C₁-C₄)haloalkylsulfonyl, and radicals of the formulae R′—C(═Z′)R′—C(═)—Z—, R′—Z—C(═Z′)—, R′R″N—C(═Z′)—, R′—Z—C(═Z′)—O—, R′R″N—C(═Z′)—Z—, R′—Z—C(═Z′)—NR″— and R′R″N—C(═Z′)—NR′″—
in which R′, R″ and R′″, in each case independently of one another, are (C₁-C₄)alkyl, phenyl, phenyl-(C₁-C₄)alkyl, (C₃-C₆)cycloalkyl or (C₃-C₆)-cycloalkyl-(C₁-C₄)alkyl, where each of the 5 lastmentioned radicals is unsubstituted or substituted by one or more radicals selected from the group consisting of halogen, hydroxyl, amino, nitro, formyl, cyano, thiocyanato, (C₁-C₄)alkoxy, (C₁-C₄)alkylthio, mono(C₁-C₄)alkylamino, di(C₁-C₄)alkylamino, (C₂-C₄)alkenyl, (C₂-C₄)alkynyl, (C₃-C₆)cycloalkyl and in the case of cyclic radicals also (C₁-C₄)alkyl and (C₁-C₄)haloalkyl, and in which Z and Z′ independently of one another are each an oxygen or sulfur atom, and, including substituents, has 1 to 20 carbon atoms.

3. A compound or a salt thereof as claimed in claim 1, wherein
R² is a cycloalkyl radical having 4 to 6 ring atoms, a bicyclic cyloalkyl radical having 4 to 6 ring atoms, a cycloalkenyl radical having 4 to 6 ring atoms, phenyl or a heterocyclic saturated or partially unsaturated radical having 3 to 6 ring atoms and one heteroatom selected from the group consisting of N, O and S, or a heteroaromatic radical having 5 or 6 ring atoms and one heteroatom selected from the group consisting of N, O and S or a heterocyclic radical having 2 to 4 heteroatoms selected from the group consisting of the heteroaromatic radicals pyrimidinyl, pyridazinyl, pyrazinyl, triazinyl, thiazolyl, thiadiazolyl, oxazolyl, isoxazolyl, pyrazolyl, imidazolyl, triazolyl and tetrazolyl or the partially unsaturated or saturated heterocyclic radicals piperazinyl, dioxolanyl, oxazolinyl, isoxazolinyl, oxazolidinyl, isoxazolidinyl and morpholinyl, where each of the cyclic radicals mentioned above for R² is unsubstituted or substituted and, including substituents, has up to 30 carbon atoms, or
is a radical of the formula —A—Z in which
A is straight-chain alkylene having 1 to 4 carbon atoms or straight-chain alkenylene or alkynylene having in each case 2 to 5 carbon atoms, where each of the three lastmentioned diradicals is unsubstituted or substituted by one or more radicals selected from the group consisting of halogen, nitro, cyano, thiocyanato, (C₁-C₄)alkyl, (C₁-C₄)alkoxy, (C₁-C₄)alkylthio, (C₁-C₄)haloalkyl and (C₁-C₄)haloalkoxy, and
Z is a cycloalkyl radical having 3 to 6 ring atoms, a bicyclic cycloalkyl radical having 4 to 6 ring atoms, a cycloalkenyl radical having 4 to 6 ring atoms, or phenyl or a heterocyclic saturated or partially unsaturated radical having 3 to 6 ring atoms and one heteroatom selected from the group consisting of N, O and S, or a heteroaromatic radical having 5 or 6 ring atoms and one heteroatom selected from the group consisting of N, O and S or a heterocyclic radical having 2 to 4 heteroatoms selected from the group consisting of the heteroaromatic radicals pyrimidinyl, pyridazinyl, pyrazinyl, triazinyl, thiazolyl, thiadiazolyl, oxazolyl, isoxazolyl, pyrazolyl, imidazolyl, triazolyl and tetrazolyl or the partially unsaturated or saturated heterocyclic radicals piperazinyl, dioxolanyl, oxazolinyl, isoxazolinyl,. oxazolidinyl, isoxazolidinyl and morpholinyl, where each of the cyclic radicals mentioned for Z is unsubstituted or substituted and, including substituents, has up to 30 carbon atoms,
where the substituents at the radicals R² and Z are in each case one or more identical or different substituents selected from the group of the radicals A), B), C) and D), where
group A) consists of the radicals halogen, hydroxyl, amino, nitro, formyl, carboxyl, aminocarbonyl, sulfo, cyano, thiocyanato and oxo,
group B) consists of the radicals (C₁-C₆)alkyl, (C₁-C₆)alkoxy, (C₁-C₆)alkylthio, mono(C₁-C₆)alkylamino, di(C₁-C₄)alkylamino, (C₂-C₆)alkenyl, (C₂-C₆)alkynyl, (C₃-C₉)cycloalkyl, (C₄-C₉)cycloalkenyl, (C₁-C₆)alkylidene, (C₄-C₉)cycloalkylidene, radicals of the formulae R^{a}—C(═Z*)—, R^{a}—C(═Z*)—Z**, R^{a}—Z**—C(═Z*)—, R^{a}R^{b}N—C(═Z*)—, R^{a}—Z**—C(═Z*)—O—, R^{a}R^{b}N—C(═Z*)—Z**—, R^{a}—Z**—C(═Z*)—NR^{b} and R^{a}R^{b}N—C(═Z*)—NR^{c}—, in which R^{a}, R^{b} and R^{c} independently of one another are each (C₁-C₆)alkyl, phenyl, phenyl-(C₁-C₆)alkyl, (C₃-C₉)cycloalkyl or (C₃-C₉)cycloalkyl-(C₁-C₆)alkyl and in which Z* and Z** independently of one another are each an oxygen or sulfur atom,
group C) consists of the radicals according to group B), but each radical is substituted by one or more radicals selected from the group consisting of halogen, hydroxyl, amino, nitro, formyl, carboxyl, sulfo, cyano, thiocyanato, (C₁-C₄)alkoxy, (C₁-C₄)haloalkoxy, (C₁-C₄)alkylthio, (C₁-₄)haloalkylthio, mono(C₁-C₄)alkylamino, di(C₁-C₄)alkylamino, (C₃-C₉)cycloalkyl, (C₄-C₉)cycloalkylene, (C₄-C₉)cycloalkylidene, [(C₁-C₄)alkyl]carbonyl, [(C₁-C₄)alkoxy]carbonyl, aminocarbonyl, mono(C₁C₄)alkylaminocarbonyl, di(C₁-C₄)alkylaminocarbonyl, phenyl, phenoxy, phenylthio, phenylcarbonyl, heterocyclyl, heterocyclyloxy, heterocyclylthio and heterocyclylamino,
where each of the 21 lastmentioned radicals is unsubstituted or substituted by one or more radicals selected from the group consisting of halogen, nitro, cyano, (C₁-C₄)alkoxy, (C₁-C₄)alkylthio, (C₁-C₄)haloalkoxy, formyl, (C₁-C₄)alkylcarbonyl and (C₁-C₄)alkoxycarbonyl and, in the case of cyclic radicals, also by (C₁-C₄)alkyl, (C₁-C₄)haloalkyl and (C₁-C₆)alkylidene and, in the case of cyclic radicals, also by (C₁-C₆)alkyl, (C₁-C₆)haloalkyl and (C₁-C₆)alkylidene, and
group D) consists of divalent or trivalent aliphatic bridges having 1 to 6 carbon atoms which, in the case of divalent bridges, connects two or, in the case of trivalent bridges, three carbon atoms of the cyclic skeleton, and the radical R² or Z thus being the radical of a bicycle or tricycle, where each of the bridges is unsubstituted or substituted by one or more substituents selected from the group consisting of halogen, nitro, cyano, (C₁-C₄)alkyl, (C₁-C₄)alkoxy, (C₁-C₄)alkylthio, (C₁-C₄)haloalkyl, (C₁-C₄)haloalkoxy, formyl, (C₁-C₄)alkylcarbonyl, (C₁-C₄)alkoxycarbonyl and oxo.

4. A compound or a salt thereof as claimed in claim 1, wherein
R³ is hydrogen, (C₁-C₄)alkyl, which is unsubstituted or substituted by one or more radicals selected from the group consisting of halogen, hydroxyl, amino, cyano, thiocyanato, (C₁-C₄)alkoxy, (C₁-C₄)haloalkoxy, (C₁-C₄)alkylthio, (C₁-C₄)haloalkylthio, mono(C₁-C₄)alkylamino and di(C₁-C₄)alkylamino, or is phenyl or (C₃-C₆)cycloalkyl, where each of the 2 lastmentioned radicals is unsubstituted or substituted by one or more radicals selected from the group consisting of halogen, hydroxyl, amino, nitro, formyl, carboxyl, sulfonyl, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl and (C₁-C₄)haloalkylsulfonyl or
a radical of the formula N(B¹—D¹)(B²—D²),
R⁴ is hydrogen, (C₁-C₄)alkyl, phenyl or (C₃-C₆)cycloalkyl, where each of the 3 lastmentioned radicals is unsubstituted or substituted by one or more radicals selected from the group consisting of halogen, hydroxyl, amino, nitro, formyl, carboxyl, sulfonyl, cyano, thiocyanato, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl, (C₁-C₄)haloalkylsulfonyl and, in the case of cyclic radicals, also by (C₁-C₄)alkyl and (C₁-C₄)haloalkyl, or is formyl, [(C₁-C₄)alkyl]carbonyl, [(C₁-C₄)alkoxy]carbonyl, aminocarbonyl, mono(C₁-C₄)alkylaminocarbonyl or di(C₁-C₄)alkylaminocarbonyl;
B¹ and B² independently of one another are each a direct bond or a divalent group of the formulae —C(═Z*)—, —C(═Z*)—Z**—, —C(═Z*)—NH— or —C(═Z*)—NR*—, where Z*=O or S, Z**=O or S and R*=(C₁-C₄)alkyl, phenyl, phenyl(C₁-C₄)alkyl, (C₃-C₆)cycloalkyl or (C₃-C₆)cycloalkyl-(C₁-C₄)alkyl, where each of the 5 lastmentioned radicals is unsubstituted or substituted by one or more radicals selected from the group consisting of halogen, hydroxyl, amino, nitro, formyl, carboxyl, sulfo, cyano, thiocyanato, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl, (C₁-C₄)haloalkylsulfonyl and, in the case of cyclic radicals, also by (C₁-C₄)alkyl and (C₁-C₄)haloalkyl; and
D¹ and D² in each case independently of one another hydrogen, (C₁-C₆)alkyl, phenyl, phenyl-(C₁-C₄)alkyl, (C₃-C₆)cycloalkyl or (C₃-C₆)cycloalkyl-(C₁-C₆)alkyl, where each of the 5 lastmentioned radicals is unsubstituted or substituted, preferably unsubstituted or substituted by one or more radicals selected from the group consisting of halogen, hydroxyl, amino, nitro, formyl, carboxyl, sulfo, cyano, thiocyanato, (C₁-C₄)alkoxy, (C₁-C₄)haloalkoxy, (C₁-C₄)alkylthio, (C₁-C₄)-haloalkylthio, mono(C₁-C₄)alkylamino, di(C₁-C₄)alkylamino, (C₃-C₉ )cycloalkyl, [(C₁-C₄)alkyl]carbonyl, [(C₁-C₄)alkoxy]carbonyl, aminocarbonyl, mono(C₁-C₄)alkylamino-carbonyl, di(C₁-C₄)alkylaminocarbonyl, (C₁-C₄)alkylsulfonyl, (C₁-C₄)haloalkylsulfonyl and, in the case of cyclic radicals, also by (C₁-C₄)alkyl and (C₁-C₄)haloalkyl.

5. A compound or a salt thereof as claimed in claim 1, wherein
(X)ₙ are n substituents X, and X, independently of the others, is in each case preferably halogen, hydroxyl, amino, nitro, formyl, carboxyl, cyano, thiocyanato, aminocarbonyl or (C₁-C₄)alkyl, (C₁-C₄)alkoxy, (C₁-C₄)alkylthio, mono(C₁-C₄)alkylamino, di(C₁-C₄)alkylamino, (C₂-C₄)alkenyl, (C₂-C₄)alkynyl, [(C₁-C₄)alkyl]carbonyl, [(C₁-C₄)alkoxy]carbonyl, mono(C₁-C₄)alkylaminocarbonyl, di(C₁-C₄)alkylaminocarbonyl, N-(C₁-C₆)alkanoylamino or N-(C₁-C₄)alkanoyl-N-(C₁-C₄)alkylamino,
where each of the 13 lastmentioned radicals is unsubstituted or substituted, preferably unsubstituted or substituted by one or more radicals selected from the group consisting of halogen, hydroxyl, amino, cyano, thiocyanato, (C₁-C₄)alkoxy, (C₁-C₄)haloalkoxy, (C₁-C₄)alkylthio, mono(C₁-C₄)alkylamino, di(C₁-C₄)alkylamino, (C₃-C₆)cycloalkyl, (C₃-C₆)cycloalkylamino, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, phenyl, phenoxy, phenylthio, phenylcarbonyl, heterocyclyl, heterocyclyloxy, heterocyclylthio and heterocyclylamino, where each of the 8 lastmentioned radicals is unsubstituted or substituted by one or more substituents selected from the group consisting of halogen, nitro, cyano, (C₁-C₄)alkyl, (C₁-C₄)alkoxy, (C₁-C₄)alkylthio, (C₁-C₄)haloalkyl, (C₁-C₄)haloalkoxy, formyl, (C₁-C₄)alkyl-carbonyl and (C₁-C₄)alkoxy-carbonyl,
or is (C₃-C₉)cycloalkyl, phenyl, phenoxy, phenylthio, phenylcarbonyl, heterocyclyl, heterocyclyloxy, heterocyclylthio or heterocyclylamino,
where each of the 9 lastmentioned radicals is unsubstituted or substituted, preferably unsubstituted or substituted by one or more radicals selected from the group consisting of halogen, hydroxyl, amino, nitro, formyl, carboxyl, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₆)cycloalkyl, [(C₁-C₄)alkyl]carbonyl, [(C₁-C₄)alkoxy]carbonyl, aminocarbonyl, mono(C₁-C₄)alkylaminocarbonyl and di(C₁-C₄)alkyl-aminocarbonyl,
or two adjacent radicals X together are a fused-on cycle having from 4 to 6 ring atoms which is carbocyclic or contains hetero ring atoms selected from the group consisting of O, S and N, and which is unsubstituted or substituted by one or more radicals selected from the group consisting of halogen, (C₁-C₄)alkyl and oxo, and
n is 0, 1, 2or 3.

6. A compound or a salt thereof as claimed in claim 1, wherein
R¹ is (C₁-C₄)alkyl, (C₁-C₄)haloalkyl, benzyl or (C₃-C₆)cycloalkyl-(C₁-C₂)alkyl,
R² is cyclobutyl, cyclopentyl or cyclohexyl, cyclobut-1-enyl, cyclobut-2-enyl, cyclopent-1-enyl, cyclopent-2-enyl, cyclopent-3-enyl, oxiranyl, oxetan-2-yl, oxetan-3-yl, oxolanyl (=tetrahydrofuryl), such as oxolan-2-yl or oxolan-3-yl, oxanyl, such as oxan-2-yl, oxan-3-yl or oxan4-yl, 1-, 2- or 3-azetidinyl, 1-, 2-or 3-azolidinyl (pyrrolidinyl), 1-, 2-, 3- or 4-piperidinyl, 2- or 3-furyl, 2- or 3-thienyl, 2-, 3- or 4-pyridyl, 2- or 3-pyrrolyl, pyrrolinyl, pyrimidinyl, pyridazinyl, pyrazinyl, triazinyl, thiazolyl, thiadiazolyl, oxazolyl, isoxazolyl, pyrazolyl, imidazolyl, triazolyl, tetrazolyl, piperazinyl, dioxolanyl, oxazolinyl, isoxazolinyl, oxazolidinyl, isoxazolidinyl or morpholinyl, where each of the cyclic radicals mentioned above for R² is unsubstituted or substituted and, including substituents, has up to 30 carbon atoms, or is a radical of the formula —A—Z in which
A is straight-chain alkylene having 1 to 4 carbon atoms or straight-chain alkenylene or alkynylene having in each case 2 to 5 carbon atoms, where each of the three lastmentioned diradicals is unsubstituted or substituted by one or more radicals selected from the group consisting of halogen, nitro, cyano, thiocyanato, (C₁-C₄)alkyl, (C₁-C₄)alkoxy, (C₁-C₄)alkylthio, (C₁-C₄)haloalkyl and (C₁-C₄)haloalkoxy, and
Z is cyclopropyl, cyclobutyl, cyclopentyl or cyclohexyl, cyclobut-1-enyl, cyclobut-2-enyl, cyclopent-1-enyl, cyclopent-2-enyl, cyclopent-3-enyl, oxiranyl, oxetan-2-yl, oxetan-3-yl, oxolanyl (=tetrahydrofuryl), such as oxolan-2-yl or oxolan-3-yl, oxanyl, such as oxan-2-yl, oxan-3-yl or oxan4-yl, 1-, 2- or 3-azetidinyl, 1-, 2- or 3-azolidinyl, 1-, 2-, 3- or 4-piperidinyl, 2- or 3-furyl, 2- or 3-thienyl, 2-, 3- or 4-pyridyl, 2- or 3-pyrrolyl, pyrrolinyl, pyrimidinyl, pyridazinyl, pyrazinyl, triazinyl, thiazolyl, thiadiazolyl, oxazolyl, isoxazolyl, pyrazolyl, imidazolyl, triazolyl, tetrazolyl, piperazinyl, dioxolanyl, oxazolinyl, isoxazolinyl, oxazolidinyl, isoxazolidinyl or morpholinyl, where each of the cyclic radicals mentioned above for Z is unsubstituted or substituted and, including substituents, has up to 30 carbon atoms,
where the substituents at the cyclic radicals R² or Z are one or more radicals selected from the group consisting of halogen, hydroxyl, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, (C₁-C₄)alkylidene, mono(C₁-C₄)alkylamino and di(C₁-C₄)alkylamino, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)-alkylsulfonyl and (C₁-C₄)haloalkylsulfonyl,
R³ is amino,
R⁴ is hydrogen,
(X)ₙ are n substituents X, and X, independently of the others, is in each case halogen, OH, NO₂, CN, SCN, (C₁-C₆)alkyl, (C₁-C₆)alkoxy, (C₁-C₄)alkylcarbonyl or (C₁-C₄)alkyloxycarbonyl, where the four lastmentioned radicals are unsubstituted or substituted by halogen or (C₁-C₄)alkoxy, and
n is 1 or 2.

7. A process for preparing a compound of the formula (I) as defined in claim 1 or a salt thereof, which comprises
a) reacting a compound of the formula (II)
R¹—Fu (II)
in which Fu is a functional group selected from the group consisting of the carboxylic esters, carboxylic orthoesters, carbonyl chlorides, carboxamides, carboxylic anhydrides and trichloromethyl with a group of the formula (III) or an acid addition salt thereof 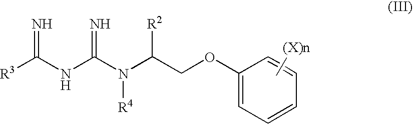
or
b) reacting a compound of the formula (IV) 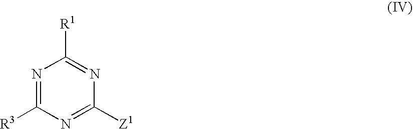
in which Z¹ is an exchangeable radical or a leaving group with a suitable amine of the formula (V) or an acid addition salt thereof 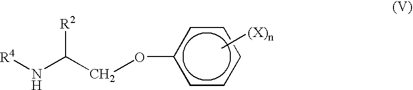
where in the formulae (II), (III), (IV) and (V) the radicals R¹, R², R³, R⁴, A¹, A² and X 1o and also n are as defined in formula (I).

8. A herbicidal or plant-growth regulating agent which comprises one or more compounds of the formula (I) or salts thereof as claimed in claim 1 and formulation auxiliaries which are customary in crop protection.

9. A method for controlling harmful plants or for regulating the growth of plants, which comprises applying an effective amount of one or more compounds of the formula (I) or salts thereof as claimed in claim 1 to the plants, to parts of plants, to plant seed or to the area under cultivation.

10. A method for controlling harmful plants or for regulating the growth of plants wherein a compound of the formula (I) or salts thereof as claimed in claim 1 is applied to in a customary manner as herbicides and plant growth regulators.
